SEQUENCE LISTING (48 sequences)

SEQ ID NO: 1 - protein, 898 aa, Escherichia coli
MNTKMNERWRTPMKLKYLSCTILAPLAIGVFSATAADNNSAIYFNTSQPINDLQGSLAAEVKFAQSQILPAHPKEGDSQPHLTSLRKSLLLVRPVKADDKTPVQVEARDDNNKILGTLTLYPPSSLPDTIYHLDGVPEGGIDFTPHNGTKKIINTVAEVNKLSDASGSSIHSHLTNNALVEIHTANGRWVRDIYLPQGPDLEGKMVRFVSSAGYSSTVFYGDRKVTLSVGNTLLFKYVNGQWFRSGELENNRITYAQHIWSAELPAHWIVPGLNLVIKQGNLSGRLNDIKIGAPGELLLHTIDIGMLTTPRDRFDFAKDKEAHREYFQTIPVSRMIVNNYAPLHLKEVMLPTGELLTDMDPGNGGWHSGTMRQRIGKELVSHGIDNANYGLNSTAGLGENSHPYVVAQLAAHNSRGNYANGIQVHGGSGGGGIVTLDSTLGNEFSHEVGHNYGLGHYVDGFKGSVHRSAENNNSTWGWDGDKKRFIPNFYPSQTNEKSCLNNQCQEPFDGHKFGFDAMAGGSPFSAANRFTMYTPNSSAIIQRFFENKAVFDSRSSTGFSKWNADTQEMEPYEHTIDRAEQITASVNELSESKMAELMAEYAVVKVHMWNGNWTRNIYIPTASADNRGSILTINHEAGYNSYLFINGDEKVVSQGYKKSFVSDGQFWKERDVVDTREARKPEQFGVPVTTLVGYYDPEGTLSSYIYPAMYGAYGFTYSDDSQNLSDNDCQLQVDTKEGQLRFRLANHRANNTVMNKFHINVPTESQPTQATLVCNNKILDTKSLTPAPEGLTYTVNGQALPAKENEGCIVSVNSGKRYCLPVGQRSGYSLPDWIVGQEVYVDSGAKAKVLLSDWDNLSYNRIGEFVGNVNPADMKKVKAWNGQYLDFSKPRSMRVVYK

SEQ ID NO: 2 - protein, 506 aa, Bacteroides thetaiotaomicron
MNRILPLLSLPLLSLAAVFAANTPEHIGNDLKLFKDSSCTSLKPDVKNTSAFQSDAMKELATKILAGHYKPDYLYAEYRALPSPRQTGKNLRIGDGFSKYDNMTGVYLEKGRHVVLVGKTEGQEISLLLPNLMRKPAEGVQPTKDPNGWGLHKKQIPLKEGINIIDVETPANAYISYFTEDAGKAPKIPVHFVTGKANGYFDTTRGDTNKDWVRLLDQAVSPIMDARGKYIQVAYPVEFLKKFTKDRGTELINAYDKLIGIQYQLMGLDKYGKIPENRVLARVNFNYYMFRDGDGVAYLGNDGTMRMVTDPENVLKGDACWGFSHEVGHVMQMRPMTWGGMTEVSNNIFSLQAAAKTGNESRLKRQGSYDKARKEIIEGEIAYLQSKDVFNKLVPLWQLHLYFTKNGHPDFYPDVMEYLRNNAGNYGGNDTVKYQFEFVKACCDVTKTDLTDFFEKWGFFKPGKFHIGDYAQYDFNVTPEMVEETKKWIAGKGYPKPETDITELSE

SEQ ID NO: 3 - protein, 840 aa, Akkermansia muciniphila
MTIKRFITNLLALFTLFTVSLACKDTEKSIINSSFSISEEYLIQNLDKSSTSVQIPINTSMELAQWSVSYEANWLQCSKQKTAAEGTFLRITVNENTGETKRTANIKVTSTTATYTITVNQYAKGEVIVEGDIKVTPTGGKASEHQEGQDIENTYDGKFSTDGAAPFHTPWGQSAKFPVTLEYYFKGDTEIDYLIYYTRSGNGNFGKVKVYTTTNPDRSDYTLQGEYDFKEQNAPSKVSFSEGIKATGIKFEVLSGLGDFVSCDEMEFYKTNTDKTLDKQLLTVFTDITCTEIKNNVTNEQIQALPDYFVRIAEAVRDNTYDKWEKEFRIRSYEPYSNIAEWADKLMTKKYSDLDNPTGISVKAGDDIIVLVGDTYGQNISMQCIWETGTEYKQTASSGDVYMLNPGVNKLTMKGEGQLFVMYNTELTSNTAKPIKIHIPLGSGTVNGFFDLKEHKTDEKYAELLKKSTHKYFCIRGEKIMFYFHRNKLLEYVPNNILSAIHLWDNIVGWQQELMGIDDVRPSQVNNHLFAISPEGSYMWASDYQIGFVYTYLGNILLEDNVMAAEDNAWGPAHEIGHVHQAAINWASSTESSNNLFSNFIIYKLGKYKSRGNGLGSVATARYANGQAWYNMGDATHQNEDTETHMRMNWQLWIYYHRCEYKTDFWQTLFKLMREVNMTEGEDPGKKQLEFAKMASKAANQNLTDFFEMWGFFEPVNTTIEQYGTYKYYVSDAMIREAKEYMAQFPAPKHAFQYIEDRKKSEFPSNDYRYSAVGDVGYYTQFKENQKITKAITAELAGRKVSIQNGDEAVAFELRENDENGKLLYFSTFTTFEIPSSILM

SEQ ID NO: 4 - protein, 382 aa, Bacteroides thetaiotaomicron
MTVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDAFYPKVGNASGAGYSCLSYRKNVDKETLYVVYEANGSIEFQDLSRHLPVIKSYN

SEQ ID NO: 5 - protein, 51 aa, synthetic construct
MDPDLEIEAAFLERENTALETRVAELRQRVQRLRNRVSQYRTRYGPLGGGK

SEQ ID NO: 6 - protein, 51 aa, synthetic construct
MDPDLEIRAAFLRQRNTALRTEVAELEQEVQRLENEVSQYETRYGPLGGGK

SEQ ID NO: 7 - protein, 35 aa, synthetic construct
GGGGSGGGGSGGGGSGGGGSGGGGSGGGGSGGGGS

SEQ ID NO: 8 - protein, 49 aa, synthetic construct
AVGQDTQEVIVVPHSLPFKVVVISAILALVVLTIISLIILIMLWQKKPR

SEQ ID NO: 9 - protein, 20 aa, synthetic construct
PDTRPAPGSTAPPAHGVTSA

SEQ ID NO: 10 - DNA, 25 bp, synthetic construct
  source 1..25: mol_type = other DNA
taatctagacccaagcttggggcag

SEQ ID NO: 11 - DNA, 27 bp, synthetic construct
  source 1..27: mol_type = other DNA
gctgtcgacgagctcggtaccaagctt

SEQ ID NO: 12 - DNA, 31 bp, synthetic construct
  source 1..31: mol_type = other DNA
taagcggatccgaattctatggcagacaatt

SEQ ID NO: 13 - DNA, 32 bp, synthetic construct
  source 1..32: mol_type = other DNA
tgctttgtacattatatcatggtatatgaagc

SEQ ID NO: 14 - DNA, 25 bp, synthetic construct
  source 1..25: mol_type = other DNA
caccgacatcaagttcaaagaactc

SEQ ID NO: 15 - DNA, 25 bp, synthetic construct
  source 1..25: mol_type = other DNA
aaacgagttctttgaacttgatgtc

SEQ ID NO: 16 - DNA, 20 bp, synthetic construct
  source 1..20: mol_type = other DNA
gtaaagcagggctacatgag

SEQ ID NO: 17 - DNA, 89 bp, synthetic construct
  source 1..89: mol_type = other DNA
ctttgggagaagatttaagccttatgtaaagcagggctactaagaattcagtggaggagcaggatatgtactaagcaaagaagccttga

SEQ ID NO: 18 - DNA, 33 bp, synthetic construct
  source 1..33: mol_type = other DNA
ggaggataatagttgtaattccagtaccaaaac

SEQ ID NO: 19 - DNA, 28 bp, synthetic construct
  source 1..28: mol_type = other DNA
tcaaaacctagagaaaaaggccaaacac

SEQ ID NO: 20 - DNA, 22 bp, synthetic construct
  source 1..22: mol_type = other DNA
agtggttaaggacttgaaactg

SEQ ID NO: 21 - DNA, 20 bp, synthetic construct
  source 1..20: mol_type = other DNA
tctactaagcggcatcattg

SEQ ID NO: 22 - DNA, 19 bp, synthetic construct
  source 1..19: mol_type = other DNA
acacgtcaaagctacttgg

SEQ ID NO: 23 - DNA, 22 bp, synthetic construct
  source 1..22: mol_type = other DNA
agtggttaaggacttgaaactg

SEQ ID NO: 24 - DNA, 27 bp, synthetic construct
  source 1..27: mol_type = other DNA
tcagtcatgacgttggtcataattatg

SEQ ID NO: 25 - DNA, 18 bp, synthetic construct
  source 1..18: mol_type = other DNA
actcattccccaatgtgg

SEQ ID NO: 26 - DNA, 18 bp, synthetic construct
  source 1..18: mol_type = other DNA
taactcgagcaccaccac

SEQ ID NO: 27 - DNA, 24 bp, synthetic construct
  source 1..24: mol_type = other DNA
atttacagtataggtaagtccttc

SEQ ID NO: 28 - DNA, 18 bp, synthetic construct
  source 1..18: mol_type = other DNA
tgagatccggctgctaac

SEQ ID NO: 29 - DNA, 26 bp, synthetic construct
  source 1..26: mol_type = other DNA
atttacagtataggtaagtccttctg

SEQ ID NO: 30 - DNA, 723 bp, synthetic construct
  source 1..723: mol_type = other DNA
caagttcaacttcaacaaagtgggccagaactcaagaaaccaggggaaactgtgaaaatttcatgtaaagcttcaggatatccatttacaaattatgggatgaattgggtcaagcaagctcccggacaaggtctcaaatggatggggtggataaatacgtccacaggtgaatccacatttgcggatgactttaaaggacggtttgattttagtctcgagacttctgctaacactgcttatctccaaattaataatctgaaatctgaagattccgcaacatatttctgtgcacggtgggaagtctatcatggttatgtcccttattggggacaaggtacaactgtaactgtatcttccggtggggggggaagtggaggaggcggcagtggtggtggcgggtctgatatacaactcacgcaatctcataaattcctttctacgtccgttggagatagagtcagcattacgtgtaaggcttcccaagatgtctataatgcagtcgcatggtaccaacagaaaccagggcaaagtcctaaattgcttatatattctgcttcttccaggtatactggtgtcccttcccgctttacggggtccggatctgggcctgattttacgtttactatttcatccgtccaagcggaagatctcgctgtctatttctgtcaacagcattttcgtaccccttttacatttggaagtggaacgaaactcgagataaaa

SEQ ID NO: 31 - DNA, 186 bp, synthetic construct
  source 1..186: mol_type = other DNA
gcacttagtaattccattatgtattttagccattttgtcccagtcttccttccagcaaaacctactacaacaccagcgccacgcccaccaacgcctgcacctacgatagcatctcaaccactcagtctccgaccagaagcgtctaggccagcggccggcggcgcggtccatacaagagggcttgat

SEQ ID NO: 32 - DNA, 210 bp, synthetic construct
  source 1..210: mol_type = other DNA
aaaccattttgggtactcgtggttgtaggtggtgttttggcttgctattccctgcttgttacagtcgcgtttattattttctgggttcgttctaaaagatctcgccttttgcattctgattatatgaatatgactccccgcagacctgggccgactagaaaacattatcaaccgtacgctcccccgcgggattttgctgcatatagaagc

SEQ ID NO: 33 - DNA, 336 bp, synthetic construct
  source 1..336: mol_type = other DNA
cgggtcaaattttcacgttccgctgatgcacccgcatatcaacaaggacaaaatcaactttataatgaactcaatctcgggcggcgcgaagagtatgatgttctcgataaaaggcggggccgagatcccgaaatgggtgggaaaccgcggagaaagaatcctcaagaggggctttacaatgagttgcaaaaggataaaatggcggaagcttattctgaaattgggatgaaaggagaacgccgcagagggaaaggtcatgatgggctctatcaagggctcagcacggcaactaaagatacttatgatgctcttcatatgcaagctctcccaccgcga

SEQ ID NO: 34 - DNA, 711 bp, synthetic construct
  source 1..711: mol_type = other DNA
atggtcagcaaaggagaggaacttataaaggaaaatatgcatatgaaattgtacatggaaggtacagtcgataatcaccattttaagtgtacctctgaaggagaggggaaaccttatgaaggaactcaaactatgaggattaaagtcgtggaaggtgggccacttccattcgcgtttgatattctcgcaacaagttttctgtatggttccaaaacctttattaatcatacgcaaggaatccctgatttctttaaacaatcatttcccgaaggctttacctgggaacgcgtgacgacgtatgaggatggtggagtcttgactgccactcaagatacttcactgcaagatgggtgtctgatttataatgtgaaaattcgcggcgtcaattttacctctaatgggccagtcatgcaaaagaagactttgggttgggaagcatttacagaaaccttgtatccagcggatggtggacttgaggggagaaatgatatggcactcaaactggttggcggaagtcacctcatagctaatgctaaaacgacttaccgctctaagaagccggccaagaatctgaaaatgcccggtgtgtattacgttgattatcggctcgagcggattaaagaagcgaataatgaaacatatgtggaacaacatgaagttgccgtcgcacggtattgtgatctgccatcaaagctcggccataaactgaac

SEQ ID NO: 35 - DNA, 2286 bp, synthetic construct
  source 1..2286: mol_type = other DNA
atggactggatatggagaatattgtttctggtgggcgctgctaccggggctcatagtcaagttcaacttcaacaaagtgggccagaactcaagaaaccaggggaaactgtgaaaatttcatgtaaagcttcaggatatccatttacaaattatgggatgaattgggtcaagcaagctcccggacaaggtctcaaatggatggggtggataaatacgtccacaggtgaatccacatttgcggatgactttaaaggacggtttgattttagtctcgagacttctgctaacactgcttatctccaaattaataatctgaaatctgaagattccgcaacatatttctgtgcacggtgggaagtctatcatggttatgtcccttattggggacaaggtacaactgtaactgtatcttccggtggggggggaagtggaggaggcggcagtggtggtggcgggtctgatatacaactcacgcaatctcataaattcctttctacgtccgttggagatagagtcagcattacgtgtaaggcttcccaagatgtctataatgcagtcgcatggtaccaacagaaaccagggcaaagtcctaaattgcttatatattctgcttcttccaggtatactggtgtcccttcccgctttacggggtccggatctgggcctgattttacgtttactatttcatccgtccaagcggaagatctcgctgtctatttctgtcaacagcattttcgtaccccttttacatttggaagtggaacgaaactcgagataaaagcacttagtaattccattatgtattttagccattttgtcccagtcttccttccagcaaaacctactacaacaccagcgccacgcccaccaacgcctgcacctacgatagcatctcaaccactcagtctccgaccagaagcgtctaggccagcggccggcggcgcggtccatacaagagggcttgataaaccattttgggtactcgtggttgtaggtggtgttttggcttgctattccctgcttgttacagtcgcgtttattattttctgggttcgttctaaaagatctcgccttttgcattctgattatatgaatatgactccccgcagacctgggccgactagaaaacattatcaaccgtacgctcccccgcgggattttgctgcatatagaagccgggtcaaattttcacgttccgctgatgcacccgcatatcaacaaggacaaaatcaactttataatgaactcaatctcgggcggcgcgaagagtatgatgttctcgataaaaggcggggccgagatcccgaaatgggtgggaaaccgcggagaaagaatcctcaagaggggctttacaatgagttgcaaaaggataaaatggcggaagcttattctgaaattgggatgaaaggagaacgccgcagagggaaaggtcatgatgggctctatcaagggctcagcacggcaactaaagatacttatgatgctcttcatatgcaagctctcccaccgcgagcttccgctacaaacttttcccttctcaaacaggctggagatgtagaggagaatccagggccgatggtcagcaaaggagaggaacttataaaggaaaatatgcatatgaaattgtacatggaaggtacagtcgataatcaccattttaagtgtacctctgaaggagaggggaaaccttatgaaggaactcaaactatgaggattaaagtcgtggaaggtgggccacttccattcgcgtttgatattctcgcaacaagttttctgtatggttccaaaacctttattaatcatacgcaaggaatccctgatttctttaaacaatcatttcccgaaggctttacctgggaacgcgtgacgacgtatgaggatggtggagtcttgactgccactcaagatacttcactgcaagatgggtgtctgatttataatgtgaaaattcgcggcgtcaattttacctctaatgggccagtcatgcaaaagaagactttgggttgggaagcatttacagaaaccttgtatccagcggatggtggacttgaggggagaaatgatatggcactcaaactggttggcggaagtcacctcatagctaatgctaaaacgacttaccgctctaagaagccggccaagaatctgaaaatgcccggtgtgtattacgttgattatcggctcgagcggattaaagaagcgaataatgaaacatatgtggaacaacatgaagttgccgtcgcacggtattgtgatctgccatcaaagctcggccataaactgaac

SEQ ID NO: 36 - DNA, 2595 bp, synthetic construct
  source 1..2595: mol_type = other DNA
gctagcgctgataataattcagccatttatttcaatacctcccagcctataaatgatctgcagggttcgttggccgcagaggtgaaatttgcacaaagccagattttacccgcccatcctaaagaaggggatagtcaaccacatctgaccagcctgcggaaaagtctgctgcttgtccgtccggtgaaagctgatgataaaacacctgttcaggtggaagcccgcgatgataataataaaattctcggtacgttaaccctttatcctccttcatcactaccggatacaatctaccatctggatggtgttccggaaggtggtatcgatttcacacctcataatggaacgaaaaagatcattaatacggtggctgaagtaaacaaactcagtgatgccagcgggagttctattcatagccatctaacaaataatgcactggtggagatccatactgcaaatggtcgttgggtaagagacatttatctgccgcagggacccgaccttgaaggtaagatggttcgctttgtttcgtctgcaggctatagttcaacggttttttatggtgatcgaaaagtcacactctcggtgggtaacactcttctgttcaaatatgtaaatggtcagtggttccgctccggtgaactggagaataatcgaatcacttatgctcagcatatttggagtgctgaactgcctgcgcactggatcgtgcctggtttaaacttggtgattaaacagggcaatctgagcggtcgcctaaatgatatcaagattggagcaccgggtgagctgttgttgcatacaattgatatcgggatgttgaccactccccgggatcgctttgattttgccaaagacaaagaagcacatagggaatatttccagaccattcctgtaagtcgtatgattgttaataattatgcgcctctacacctaaaggaagttatgttaccaaccggagagttattgacagatatggatccaggaaatggtgggtggcatagtggtacaatgcgtcaaagaataggtaaagaattggtttcgcatggcattgataatgctaactatggtttaaatagtaccgcaggcttaggggagaatagtcatccatatgtagttgcgcaattagcggcacataatagccgcggtaattatgctaatggcatccaggttcatggtggctccggaggtgggggaattgttactttagattccacattggggaatgagttcagtcatgaagttggtcataattatggtcttggtcattatgtagatggtttcaagggttctgtacatcgtagtgcagaaaataacaactcaacttggggatgggatggtgataaaaaacggtttattcctaacttttatccgtctcaaacaaatgaaaagagttgtctgaataatcagtgtcaagaaccgtttgatggacacaaatttggttttgacgccatggcgggaggcagccctttctctgctgcaaaccgtttcacaatgtatactccgaattcatcggctatcatccagcgtttttttgaaaataaagctgtgttcgatagccgttcctccaccggcttcagcaagtggaatgcagatacgcaggaaatggaaccgtatgaacacaccattgaccgtgcggagcagattacggcttcagtcaatgagctaagtgaaagcaaaatggctgagctgatggcagagtacgctgtcgtcaaagtgcatatgtggaacggtaactggacaagaaacatctatatccctacagcctccgcagataatagaggcagtatcctgaccatcaaccatgaggccggttataatagttatctgtttataaatggtgacgaaaaggtcgtttcccaggggtataaaaagagctttgtttccgatggtcagttctggaaagaacgtgatgtggttgatactcgtgaagcgcgtaagccagagcagtttggtgttcctgtgacgaccctggtggggtattacgatccggaaggcacgctgtcaagctacatctatcctgcgatgtatggtgcctatggcttcacttattccgatgatagtcagaatctatccgataacgactgccagctgcaggtggatacgaaagaagggcagttgcgattcagactggctaatcaccgggctaacaacactgtaatgaataagttccatattaacgtgccaacagaaagtcagcccacacaggccacattggtttgcaataacaagatactggataccaaatcgctcacacctgcgccagaaggacttacctatactgtaaatgggcaggcacttccagcaaaagaaaacgagggatgcatcgtgtccgtgaattcaggtaaacgttactgtttgccggttggtcaacggtcaggatatagccttcctgactggattgttgggcaggaagtctatgtcgacagcggggctaaagcgaaagtgctgctttctgactgggataacctgtcctataacaggattggtgagtttgtaggtaatgtgaacccagctgatatgaaaaaagttaaagcctggaacggacagtatttggacttcagtaaacctaggtcaatgagggttgtatataaa

SEQ ID NO: 37 - DNA, 2289 bp, synthetic construct
  source 1..2289: mol_type = other DNA
gctagcgctgataataattcagccatttatttcaatacctcccagcctataaatgatctgcagggttcgttggccgcagaggtgaaatttgcacaaagccagattttacccgcccatcctaaagaaggggatagtcaaccacatctgaccagcctgcggaaaagtctgctgcttgtccgtccggtgaaagctgatgataaaacacctgttcaggtggaagcccgcgatgataataataaaattctcggtacgttaaccctttatcctccttcatcactaccggatacaatctaccatctggatggtgttccggaaggtggtatcgatttcacacctcataatggaacgaaaaagatcattaatacggtggctgaagtaaacaaactcagtgatgccagcgggagttctattcatagccatctaacaaataatgcactggtggagatccatactgcaaatggtcgttgggtaagagacatttatctgccgcagggacccgaccttgaaggtaagatggttcgctttgtttcgtctgcaggctatagttcaacggttttttatggtgatcgaaaagtcacactctcggtgggtaacactcttctgttcaaatatgtaaatggtcagtggttccgctccggtgaactggagaataatcgaatcacttatgctcagcatatttggagtgctgaactgcctgcgcactggatcgtgcctggtttaaacttggtgattaaacagggcaatctgagcggtcgcctaaatgatatcaagattggagcaccgggtgagctgttgttgcatacaattgatatcgggatgttgaccactccccgggatcgctttgattttgccaaagacaaagaagcacatagggaatatttccagaccattcctgtaagtcgtatgattgttaataattatgcgcctctacacctaaaggaagttatgttaccaaccggagagttattgacagatatggatccaggaaatggtgggtggcatagtggtacaatgcgtcaaagaataggtaaagaattggtttcgcatggcattgataatgctaactatggtttaaatagtaccgcaggcttaggggagaatagtcatccatatgtagttgcgcaattagcggcacataatagccgcggtaattatgctaatggcatccaggttcatggtggctccggaggtgggggaattgttactttagattccacattggggaatgagttcagtcatgaagttggtcataattatggtcttggtcattatgtagatggtttcaagggttctgtacatcgtagtgcagaaaataacaactcaacttggggatgggatggtgataaaaaacggtttattcctaacttttatccgtctcaaacaaatgaaaagagttgtctgaataatcagtgtcaagaaccgtttgatggacacaaatttggttttgacgccatggcgggaggcagccctttctctgctgcaaaccgtttcacaatgtatactccgaattcatcggctatcatccagcgtttttttgaaaataaagctgtgttcgatagccgttcctccaccggcttcagcaagtggaatgcagatacgcaggaaatggaaccgtatgaacacaccattgaccgtgcggagcagattacggcttcagtcaatgagctaagtgaaagcaaaatggctgagctgatggcagagtacgctgtcgtcaaagtgcatatgtggaacggtaactggacaagaaacatctatatccctacagcctccgcagataatagaggcagtatcctgaccatcaaccatgaggccggttataatagttatctgtttataaatggtgacgaaaaggtcgtttcccaggggtataaaaagagctttgtttccgatggtcagttctggaaagaacgtgatgtggttgatactcgtgaagcgcgtaagccagagcagtttggtgttcctgtgacgaccctggtggggtattacgatccggaaggcacgctgtcaagctacatctatcctgcgatgtatggtgcctatggcttcacttattccgatgatagtcagaatctatccgataacgactgccagctgcaggtggatacgaaagaagggcagttgcgattcagactggctaatcaccgggctaacaacactgtaatgaataagttccatattaacgtgccaacagaaagtcagcccacacaggccacattggtttgcaataacaagatactggataccaaatcgctcacacctgcgccagaaggacttacctatactgtaaat

SEQ ID NO: 38 - DNA, 1458 bp, synthetic construct
  source 1..1458: mol_type = other DNA
gcgaacacgccggagcatatcgggaatgatttaaaactgtttaaagattcatcgtgtacctctttgaagccggacgtaaaaaatactagtgcctttcagagtgatgcgatgaaggaattggccacaaagatccttgccgggcattacaaacctgattatctttacgcggagtatcgcgcactgccgagtcctcggcagacagggaaaaatcttcgcatcggtgacgggtttagtaaatatgacaacatgaccggtgtgtacttggagaaaggccggcacgtcgtgttggttgggaagacagagggccaggagatttccttactgttgccgaacttgatgcggaagccggcagaaggcgtccagcctaccaaggacccgaacggctggggcttgcacaaaaaacaaatcccactcaaggagggtatcaacatcatcgacgtagagaccccggcaaatgcatatatctcatatttcactgaagatgccgggaaagcaccgaagatccctgtacacttcgttacgggcaaagctaacggctattttgacaccacccgtggggacacgaacaaagactgggtgcgtctcttggatcaagctgtcagcccgatcatggatgcacggggtaaatatattcaagtcgcatatccagtggagttccttaaaaagttcacaaaagatcgggggactgagctgattaacgcatacgacaagcttatcgggatccagtatcagctgatgggcttagataaatatggtaaaattccggaaaatcgcgtattggcacgcgtaaattttaactactatatgtttcgtgatggtgacggggtagcctatctcggcaacgatggtaccatgcgtatggtcacggacccagagaatgtattgaagggtgacgcctgctggggcttctcgcacgaggtgggtcatgttatgcagatgcggccgatgacatggggtggtatgaccgaggtgtccaacaatatctttagcttgcaagcggcagcaaagactggcaatgagtctcgtttaaaacggcagggtagctacgacaaagctcgcaaggaaattattgagggtgaaatcgcgtatttgcaatcgaaagacgtttttaacaagttggtaccgttatggcaattacatctgtactttacgaagaacggccatccagatttctatccagatgtaatggaatatctgcgtaataacgctggcaattatggtggtaacgacacagtcaaatatcagttcgagttcgttaaagcgtgttgcgatgtcacgaagaccgatcttacggatttttttgaaaaatggggcttctttaagcctgggaaatttcacatcggtgattacgcccagtatgactttaacgtaacaccagagatggttgaagagaccaaaaaatggattgctgggaaggggtacccaaaacctgaaactgacatcacggagttgtctgaa

SEQ ID NO: 39 - DNA, 2469 bp, synthetic construct
  source 1..2469: mol_type = other DNA
ttttcaatttccgaggagtatttaattcaaaatttagacaagtcctccacatccgtacaaatccctattaatacttcaatggagcttgcgcagtggtccgtgtcgtacgaagcgaattggttacaatgctctaagcaaaaaacagccgcggaagggacgtttctccgcattacggtgaacgaaaatactggggagacaaagcggacggctaatatcaaggtcacctcaacaacagccacttacaccattacagtgaatcaatatgccaaaggtgaagtaattgtagaaggtgatattaaggtgactcctacggggggcaaagcttcggagcaccaagagggtcaggatattgagaacacttatgatggtaaatttagtacagatggtgctgccccattccacacgccgtgggggcagtccgcaaagtttccagttactctggaatactacttcaagggcgacactgagattgattacttgatttactatacgcgttcaggcaatggcaatttcggtaaggtgaaggtgtataccacaacaaatccagaccggtcggactatacccttcaaggggaatacgactttaaggagcaaaacgcaccttcaaaagtctcgttttcagaagggattaaggcgactggcattaagtttgaggtgttatctgggttgggtgatttcgtatcctgtgacgagatggaattttataaaacgaacactgacaagacgttggacaaacaattgttaactgtctttaccgacattacctgtactgagattaagaataatgtgacaaacgaacaaattcaggctttaccagattatttcgtgcgtatcgccgaggcggtgcgggataatacttatgacaaatgggagaaggagttccgtatccgcagttacgagccttacagtaacattgctgagtgggctgacaaacttatgactaaaaaatattcagatttggacaatccgacaggcatctcagtgaaggctggcgatgatatcatcgttctcgtgggcgacacttacgggcagaatatctctatgcagtgcatttgggagacggggactgagtacaagcagacagctagcagcggggacgtatatatgctgaacccgggggtaaacaaactgacaatgaagggcgagggtcagttgttcgttatgtataatactgaattaacttctaacactgctaagccaatcaaaattcatatcccattggggtccgggacggtaaacgggtttttcgatttaaaggagcataaaactgatgagaaatacgccgaacttttgaaaaaatcgacacacaagtatttctgtatccgtggggagaagatcatgttctatttccaccgcaataagcttttagaatacgtcccaaacaacattctttctgcgatccacctgtgggataacatcgtaggttggcagcaagagcttatgggcattgacgatgtgcgcccatcgcaggtcaacaaccatctgttcgcgatctcgccagaaggtagttacatgtgggccagtgattatcagattggcttcgtttacacttacttaggtaatattttgctcgaggacaatgttatggcagcagaagacaatgcttggggtccagcacacgagatcggccatgttcatcaagctgcgattaattgggcctccagtacagaatcatcaaacaatcttttctctaacttcatcatctataagctgggcaagtacaagagtcggggcaatggtctgggttcggtggcaacagcgcggtacgctaatgggcaggcgtggtacaacatgggtgacgccactcaccagaatgaggacacggaaacacatatgcggatgaattggcaattatggatctattaccatcgctgtgaatacaagactgacttttggcaaacgttgttcaagcttatgcgggaagtcaacatgacagagggcgaagacccaggcaaaaagcaattagaatttgcaaagatggcgtccaaggccgccaaccagaatcttacagatttcttcgaaatgtggggcttttttgagccggtcaatacaactattgagcaatacgggacgtataaatactacgtctctgatgccatgatccgggaagctaaggaatacatggcacaatttccggcgcctaagcacgcattccaatatatcgaggaccgcaagaagtccgagttcccttcgaacgactatcgctactcggccgttggtgatgtcggctattatacccagtttaaagaaaatcaaaagattaccaaggcgattacggctgaactggcagggcgcaaggtttccatccagaatggcgacgaagcggttgcctttgagttacgggaaaatgacgagaacggcaagttattgtattttagcactttcaccacatttgaaatccctagttccattctcatggtgaatgcgaagttatatgccgtacaggcggacggcaaacgtatcctcctg

SEQ ID NO: 40 - DNA, 1143 bp, synthetic construct
  source 1..1143: mol_type = other DNA
accgtagagaagtcagtggtgttcaaagccgaaggcgagcattttaccgatcagaaaggaaataccattgtgggttctgggtcgggtgggactacaaaatatttccgtatcccggcaatgtgtaccacatcaaagggtacgatcgttgttttcgctgacgcccgccataatactgcgtctgatcaaagtttcatcgatacagccgccgcacgtagcacggacggcgggaaaacttggaataaaaaaatcgctatctacaatgaccgcgtcaatagcaagctgtctcgtgttatggatcctacttgcattgtcgctaatattcagggtcgcgagaccatcttagttatggtaggaaagtggaacaacaatgacaaaacttggggtgcttatcgcgacaaagcccctgatactgattgggatctggttttatacaagtcaaccgacgacggagtaactttcagcaaggttgaaactaatattcacgatatcgtaaccaaaaatggaacaatttcagcaatgttaggtggcgtagggagcggattacaacttaacgacggaaaactggtcttcccggtacaaatggtccgcacaaaaaatatcacgactgtcttgaacacctcctttatctactcaaccgacgggatcacctggagtttaccttctggatactgcgagggtttcggctccgaaaacaacatcattgaattcaatgcgtctttggtcaacaacatccgcaattcagggttgcgccgttcgtttgagacaaaggacttcgggaagacctggacggagtttccgccgatggacaaaaaagtggacaatcgtaatcatggcgtccaaggcagcactattacgattccttcaggaaacaaactggtggctgcccacagttctgcccagaataaaaacaatgattacactcgctctgatatttcactgtacgctcataacctttattcaggagaggttaaattgattgacgcattttacccgaaggtcggcaacgcatctggggccgggtatagctgtttgagctaccgcaaaaacgttgacaaggagaccttgtatgtagtgtacgaagcaaatggctctattgagttccaagacctgtcgcgccatctgcccgtcattaagtcgtacaac

SEQ ID NO: 41 - DNA, 153 bp, synthetic construct
  source 1..153: mol_type = other DNA
atggaccctgatcttgagattgaagctgccttccttgaacgggagaacacggccttggaaacgagagtggcggagttaagacaaagagtccagcgccttcgtaaccgcgtaagccagtatcggacccggtacggaccccttggcgggggaaag

SEQ ID NO: 42 - DNA, 153 bp, synthetic construct
  source 1..153: mol_type = other DNA
atggaccctgatctcgaaattcgcgcggccttcctcaggcagcgaaataccgctttgagaacggaagtcgctgagctcgaacaagaggtccagagactggagaacgaagtgagccaatatgaaacacgatatggccccctcggcggcggaaag

SEQ ID NO: 43 - DNA, 2547 bp, synthetic construct
  source 1..2547: mol_type = other DNA
gctagcgctgataataattcagccatttatttcaatacctcccagcctataaatgatctgcagggttcgttggccgcagaggtgaaatttgcacaaagccagattttacccgcccatcctaaagaaggggatagtcaaccacatctgaccagcctgcggaaaagtctgctgcttgtccgtccggtgaaagctgatgataaaacacctgttcaggtggaagcccgcgatgataataataaaattctcggtacgttaaccctttatcctccttcatcactaccggatacaatctaccatctggatggtgttccggaaggtggtatcgatttcacacctcataatggaacgaaaaagatcattaatacggtggctgaagtaaacaaactcagtgatgccagcgggagttctattcatagccatctaacaaataatgcactggtggagatccatactgcaaatggtcgttgggtaagagacatttatctgccgcagggacccgaccttgaaggtaagatggttcgctttgtttcgtctgcaggctatagttcaacggttttttatggtgatcgaaaagtcacactctcggtgggtaacactcttctgttcaaatatgtaaatggtcagtggttccgctccggtgaactggagaataatcgaatcacttatgctcagcatatttggagtgctgaactgcctgcgcactggatcgtgcctggtttaaacttggtgattaaacagggcaatctgagcggtcgcctaaatgatatcaagattggagcaccgggtgagctgttgttgcatacaattgatatcgggatgttgaccactccccgggatcgctttgattttgccaaagacaaagaagcacatagggaatatttccagaccattcctgtaagtcgtatgattgttaataattatgcgcctctacacctaaaggaagttatgttaccaaccggagagttattgacagatatggatccaggaaatggtgggtggcatagtggtacaatgcgtcaaagaataggtaaagaattggtttcgcatggcattgataatgctaactatggtttaaatagtaccgcaggcttaggggagaatagtcatccatatgtagttgcgcaattagcggcacataatagccgcggtaattatgctaatggcatccaggttcatggtggctccggaggtgggggaattgttactttagattccacattggggaatgagttcagtcatgaagttggtcataattatggtcttggtcattatgtagatggtttcaagggttctgtacatcgtagtgcagaaaataacaactcaacttggggatgggatggtgataaaaaacggtttattcctaacttttatccgtctcaaacaaatgaaaagagttgtctgaataatcagtgtcaagaaccgtttgatggacacaaatttggttttgacgccatggcgggaggcagccctttctctgctgcaaaccgtttcacaatgtatactccgaattcatcggctatcatccagcgtttttttgaaaataaagctgtgttcgatagccgttcctccaccggcttcagcaagtggaatgcagatacgcaggaaatggaaccgtatgaacacaccattgaccgtgcggagcagattacggcttcagtcaatgagctaagtgaaagcaaaatggctgagctgatggcagagtacgctgtcgtcaaagtgcatatgtggaacggtaactggacaagaaacatctatatccctacagcctccgcagataatagaggcagtatcctgaccatcaaccatgaggccggttataatagttatctgtttataaatggtgacgaaaaggtcgtttcccaggggtataaaaagagctttgtttccgatggtcagttctggaaagaacgtgatgtggttgatactcgtgaagcgcgtaagccagagcagtttggtgttcctgtgacgaccctggtggggtattacgatccggaaggcacgctgtcaagctacatctatcctgcgatgtatggtgcctatggcttcacttattccgatgatagtcagaatctatccgataacgactgccagctgcaggtggatacgaaagaagggcagttgcgattcagactggctaatcaccgggctaacaacactgtaatgaataagttccatattaacgtgccaacagaaagtcagcccacacaggccacattggtttgcaataacaagatactggataccaaatcgctcacacctgcgccagaaggacttacctatactgtaaatggtggaggcggttctggcggtggcggctcaggtggaggtggctctggcggaggaggtagcggaggtggcggtagcggcggaggaggttctggtggaggaggtagcatggaccctgatcttgagattgaagctgccttccttgaacgggagaacacggccttggaaacgagagtggcggagttaagacaaagagtccagcgccttcgtaaccgcgtaagccagtatcggacccggtacggaccccttggcgggggaaag

SEQ ID NO: 44 - DNA, 1716 bp, synthetic construct
  source 1..1716: mol_type = other DNA
gcgaacacgccggagcatatcgggaatgatttaaaactgtttaaagattcatcgtgtacctctttgaagccggacgtaaaaaatactagtgcctttcagagtgatgcgatgaaggaattggccacaaagatccttgccgggcattacaaacctgattatctttacgcggagtatcgcgcactgccgagtcctcggcagacagggaaaaatcttcgcatcggtgacgggtttagtaaatatgacaacatgaccggtgtgtacttggagaaaggccggcacgtcgtgttggttgggaagacagagggccaggagatttccttactgttgccgaacttgatgcggaagccggcagaaggcgtccagcctaccaaggacccgaacggctggggcttgcacaaaaaacaaatcccactcaaggagggtatcaacatcatcgacgtagagaccccggcaaatgcatatatctcatatttcactgaagatgccgggaaagcaccgaagatccctgtacacttcgttacgggcaaagctaacggctattttgacaccacccgtggggacacgaacaaagactgggtgcgtctcttggatcaagctgtcagcccgatcatggatgcacggggtaaatatattcaagtcgcatatccagtggagttccttaaaaagttcacaaaagatcgggggactgagctgattaacgcatacgacaagcttatcgggatccagtatcagctgatgggcttagataaatatggtaaaattccggaaaatcgcgtattggcacgcgtaaattttaactactatatgtttcgtgatggtgacggggtagcctatctcggcaacgatggtaccatgcgtatggtcacggacccagagaatgtattgaagggtgacgcctgctggggcttctcgcacgaggtgggtcatgttatgcagatgcggccgatgacatggggtggtatgaccgaggtgtccaacaatatctttagcttgcaagcggcagcaaagactggcaatgagtctcgtttaaaacggcagggtagctacgacaaagctcgcaaggaaattattgagggtgaaatcgcgtatttgcaatcgaaagacgtttttaacaagttggtaccgttatggcaattacatctgtactttacgaagaacggccatccagatttctatccagatgtaatggaatatctgcgtaataacgctggcaattatggtggtaacgacacagtcaaatatcagttcgagttcgttaaagcgtgttgcgatgtcacgaagaccgatcttacggatttttttgaaaaatggggcttctttaagcctgggaaatttcacatcggtgattacgcccagtatgactttaacgtaacaccagagatggttgaagagaccaaaaaatggattgctgggaaggggtacccaaaacctgaaactgacatcacggagttgtctgaaggtggaggcggttctggcggtggcggctcaggtggaggtggctctggcggaggaggtagcggaggtggcggtagcggcggaggaggttctggtggaggaggtagcatggaccctgatcttgagattgaagctgccttccttgaacgggagaacacggccttggaaacgagagtggcggagttaagacaaagagtccagcgccttcgtaaccgcgtaagccagtatcggacccggtacggaccccttggcgggggaaag

SEQ ID NO: 45 - DNA, 2727 bp, synthetic construct
  source 1..2727: mol_type = other DNA
ttttcaatttccgaggagtatttaattcaaaatttagacaagtcctccacatccgtacaaatccctattaatacttcaatggagcttgcgcagtggtccgtgtcgtacgaagcgaattggttacaatgctctaagcaaaaaacagccgcggaagggacgtttctccgcattacggtgaacgaaaatactggggagacaaagcggacggctaatatcaaggtcacctcaacaacagccacttacaccattacagtgaatcaatatgccaaaggtgaagtaattgtagaaggtgatattaaggtgactcctacggggggcaaagcttcggagcaccaagagggtcaggatattgagaacacttatgatggtaaatttagtacagatggtgctgccccattccacacgccgtgggggcagtccgcaaagtttccagttactctggaatactacttcaagggcgacactgagattgattacttgatttactatacgcgttcaggcaatggcaatttcggtaaggtgaaggtgtataccacaacaaatccagaccggtcggactatacccttcaaggggaatacgactttaaggagcaaaacgcaccttcaaaagtctcgttttcagaagggattaaggcgactggcattaagtttgaggtgttatctgggttgggtgatttcgtatcctgtgacgagatggaattttataaaacgaacactgacaagacgttggacaaacaattgttaactgtctttaccgacattacctgtactgagattaagaataatgtgacaaacgaacaaattcaggctttaccagattatttcgtgcgtatcgccgaggcggtgcgggataatacttatgacaaatgggagaaggagttccgtatccgcagttacgagccttacagtaacattgctgagtgggctgacaaacttatgactaaaaaatattcagatttggacaatccgacaggcatctcagtgaaggctggcgatgatatcatcgttctcgtgggcgacacttacgggcagaatatctctatgcagtgcatttgggagacggggactgagtacaagcagacagctagcagcggggacgtatatatgctgaacccgggggtaaacaaactgacaatgaagggcgagggtcagttgttcgttatgtataatactgaattaacttctaacactgctaagccaatcaaaattcatatcccattggggtccgggacggtaaacgggtttttcgatttaaaggagcataaaactgatgagaaatacgccgaacttttgaaaaaatcgacacacaagtatttctgtatccgtggggagaagatcatgttctatttccaccgcaataagcttttagaatacgtcccaaacaacattctttctgcgatccacctgtgggataacatcgtaggttggcagcaagagcttatgggcattgacgatgtgcgcccatcgcaggtcaacaaccatctgttcgcgatctcgccagaaggtagttacatgtgggccagtgattatcagattggcttcgtttacacttacttaggtaatattttgctcgaggacaatgttatggcagcagaagacaatgcttggggtccagcacacgagatcggccatgttcatcaagctgcgattaattgggcctccagtacagaatcatcaaacaatcttttctctaacttcatcatctataagctgggcaagtacaagagtcggggcaatggtctgggttcggtggcaacagcgcggtacgctaatgggcaggcgtggtacaacatgggtgacgccactcaccagaatgaggacacggaaacacatatgcggatgaattggcaattatggatctattaccatcgctgtgaatacaagactgacttttggcaaacgttgttcaagcttatgcgggaagtcaacatgacagagggcgaagacccaggcaaaaagcaattagaatttgcaaagatggcgtccaaggccgccaaccagaatcttacagatttcttcgaaatgtggggcttttttgagccggtcaatacaactattgagcaatacgggacgtataaatactacgtctctgatgccatgatccgggaagctaaggaatacatggcacaatttccggcgcctaagcacgcattccaatatatcgaggaccgcaagaagtccgagttcccttcgaacgactatcgctactcggccgttggtgatgtcggctattatacccagtttaaagaaaatcaaaagattaccaaggcgattacggctgaactggcagggcgcaaggtttccatccagaatggcgacgaagcggttgcctttgagttacgggaaaatgacgagaacggcaagttattgtattttagcactttcaccacatttgaaatccctagttccattctcatggtgaatgcgaagttatatgccgtacaggcggacggcaaacgtatcctcctgggtggaggcggttctggcggtggcggctcaggtggaggtggctctggcggaggaggtagcggaggtggcggtagcggcggaggaggttctggtggaggaggtagcatggaccctgatcttgagattgaagctgccttccttgaacgggagaacacggccttggaaacgagagtggcggagttaagacaaagagtccagcgccttcgtaaccgcgtaagccagtatcggacccggtacggaccccttggcgggggaaag

SEQ ID NO: 46 - DNA, 1401 bp, synthetic construct
  source 1..1401: mol_type = other DNA
accgtagagaagtcagtggtgttcaaagccgaaggcgagcattttaccgatcagaaaggaaataccattgtgggttctgggtcgggtgggactacaaaatatttccgtatcccggcaatgtgtaccacatcaaagggtacgatcgttgttttcgctgacgcccgccataatactgcgtctgatcaaagtttcatcgatacagccgccgcacgtagcacggacggcgggaaaacttggaataaaaaaatcgctatctacaatgaccgcgtcaatagcaagctgtctcgtgttatggatcctacttgcattgtcgctaatattcagggtcgcgagaccatcttagttatggtaggaaagtggaacaacaatgacaaaacttggggtgcttatcgcgacaaagcccctgatactgattgggatctggttttatacaagtcaaccgacgacggagtaactttcagcaaggttgaaactaatattcacgatatcgtaaccaaaaatggaacaatttcagcaatgttaggtggcgtagggagcggattacaacttaacgacggaaaactggtcttcccggtacaaatggtccgcacaaaaaatatcacgactgtcttgaacacctcctttatctactcaaccgacgggatcacctggagtttaccttctggatactgcgagggtttcggctccgaaaacaacatcattgaattcaatgcgtctttggtcaacaacatccgcaattcagggttgcgccgttcgtttgagacaaaggacttcgggaagacctggacggagtttccgccgatggacaaaaaagtggacaatcgtaatcatggcgtccaaggcagcactattacgattccttcaggaaacaaactggtggctgcccacagttctgcccagaataaaaacaatgattacactcgctctgatatttcactgtacgctcataacctttattcaggagaggttaaattgattgacgcattttacccgaaggtcggcaacgcatctggggccgggtatagctgtttgagctaccgcaaaaacgttgacaaggagaccttgtatgtagtgtacgaagcaaatggctctattgagttccaagacctgtcgcgccatctgcccgtcattaagtcgtacaacggtggaggcggttctggcggtggcggctcaggtggaggtggctctggcggaggaggtagcggaggtggcggtagcggcggaggaggttctggtggaggaggtagcatggaccctgatcttgagattgaagctgccttccttgaacgggagaacacggccttggaaacgagagtggcggagttaagacaaagagtccagcgccttcgtaaccgcgtaagccagtatcggacccggtacggaccccttggcgggggaaag

SEQ ID NO: 47 - DNA, 2175 bp, synthetic construct
  source 1..2175: mol_type = other DNA
atggagacagacacactcctgctatgggtactgctgctctgggttccaggttccactggtgactcaagattggaggaggaattgcggagacgattgaccgagccaaccgtagagaagtcagtggtgttcaaagccgaaggcgagcattttaccgatcagaaaggaaataccattgtgggttctgggtcgggtgggactacaaaatatttccgtatcccggcaatgtgtaccacatcaaagggtacgatcgttgttttcgctgacgcccgccataatactgcgtctgatcaaagtttcatcgatacagccgccgcacgtagcacggacggcgggaaaacttggaataaaaaaatcgctatctacaatgaccgcgtcaatagcaagctgtctcgtgttatggatcctacttgcattgtcgctaatattcagggtcgcgagaccatcttagttatggtaggaaagtggaacaacaatgacaaaacttggggtgcttatcgcgacaaagcccctgatactgattgggatctggttttatacaagtcaaccgacgacggagtaactttcagcaaggttgaaactaatattcacgatatcgtaaccaaaaatggaacaatttcagcaatgttaggtggcgtagggagcggattacaacttaacgacggaaaactggtcttcccggtacaaatggtccgcacaaaaaatatcacgactgtcttgaacacctcctttatctactcaaccgacgggatcacctggagtttaccttctggatactgcgagggtttcggctccgaaaacaacatcattgaattcaatgcgtctttggtcaacaacatccgcaattcagggttgcgccgttcgtttgagacaaaggacttcgggaagacctggacggagtttccgccgatggacaaaaaagtggacaatcgtaatcatggcgtccaaggcagcactattacgattccttcaggaaacaaactggtggctgcccacagttctgcccagaataaaaacaatgattacactcgctctgatatttcactgtacgctcataacctttattcaggagaggttaaattgattgacgcattttacccgaaggtcggcaacgcatctggggccgggtatagctgtttgagctaccgcaaaaacgttgacaaggagaccttgtatgtagtgtacgaagcaaatggctctattgagttccaagacctgtcgcgccatctgcccgtcattaagtcgtacaacttggaagaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtactagtggttctggttctcctgcaggaatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaa

SEQ ID NO: 48 - DNA, 1176 bp, synthetic construct
  source 1..1176: mol_type = other DNA
atggagacagacacactcctgctatgggtactgctgctctgggttccaggttccactggtgactcaagattggaggaggaattgcggagacgattgaccgagccaatggaccctgatctcgaaattcgcgcggccttcctcaggcagcgaaataccgctttgagaacggaagtcgctgagctcgaacaagaggtccagagactggagaacgaagtgagccaatatgaaacacgatatggccccctcggcggcggaaagttggaagaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtactagtggttctggttctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaa

(19) United States
(12) Patent Application Publication
(10) Pub. No.: US 2025/0152627 A1
(43) Pub. Date: May 15, 2025

(54) COMPOSITIONS AND METHODS FOR ENHANCING IMMUNE CELL FUNCTION USING MODIFIED CELLS WITH GLYCOCALYX EDITING ENZYMES

(71) Applicant: Cornell University, Ithaca, NY (US)

(72) Inventors: Matthew PASZEK, Lansing, NY (US); Sangwoo PARK, Ithaca, NY (US)

(21) Appl. No.: 18/715,695

(22) PCT Filed: Dec. 5, 2022
(86) PCT No.: PCT/US2022/080937

§ 371 Date: May 31, 2024

Related U.S. Application Data
(60) Provisional application No. 63/285,947, filed on Dec. 3, 2021.

(51) Int. Cl. A61K 35/17; A61K 38/47; A61K 38/48; C12N 5/0783; C12N 5/0784; C12N 5/0786; C12N 9/24; C12N 9/64

(52) U.S. Cl. CPC A61K 35/17; A61K 38/47; A61K 38/4886; C12N 5/0636; C12N 5/0639; C12N 5/0645; C12N 5/0646; C12N 9/2402; C12N 9/6416; C12N 2510/00

(57) ABSTRACT

Provided are modified cells that can alter the glycocalyx of cancer cells. The modified cells include modified lymphocytes that have a one or more plasma membrane-coupled glycocalyx-editing enzymes. The modified lymphocytes include modified immune cells. The modified immune cells include T cells, natural killer cells, macrophages and dendritic cells. The modified immune cells may also express a chimeric antigen receptor. Methods of using the modified cells to alter the glycocalyx of cancer cells, and for cancer therapy, are also provided.

### CROSS REFERENCE TO RELATED APPLICATIONS

This application claims priority to U.S. provisional patent application No. 63/285,947, filed Dec. 3, 2021, the entire disclosure of which is incorporated herein by reference.

### SEQUENCE LISTING

The instant application contains a Sequence Listing which has been submitted in .xml format and is hereby incorporated by reference in its entirety. Said .xml copy was created on Dec. 5, 2022, is named “018617_01389_ST26.XML”, and is 72,267 bytes in size.

### BACKGROUND

Tumor cells construct a glycocalyx with physical and biochemical attributes that guard against detection and destruction by surveilling immune cells¹. Essentially all types of human cancers exhibit changes in glycan synthesis, resulting in their expression at atypical levels or with altered structural attributes, phenomena that were first observed more than six decades ago²⁻⁴. Immunomodulatory glycans can reprogram the activities of important immune cell types, including macrophages, dendritic cells, cytotoxic T-cells, and Natural Killer (NK) cells⁵. Larger macromolecules in the glycocalyx also are proposed to sterically shield molecular epitopes, fundamentally changing how immune cells perceive and interact with tumor cells¹. While a multifaceted role for the cancer-associated glycocalyx in immune evasion has become clear, a physical understanding of the glycocalyx remains limited, especially regarding cancer immunotherapy. With the rapid advancement of adoptive cell therapies for cancer, identification of strategies to overcome the immune-protective actions of the cancer cell glycocalyx have become of strong interest.

Cancer cells often generate a glycocalyx with high levels of cell surface mucins, which can directly modulate the tumor immune microenvironment⁶⁻¹⁰ Mucin-type O-glycans initiate with N-acetylgalactosamine (GalNAc) that is linked to serine or threonine on the mucin polypeptide backbone and elongated into more complex structures through the sequential actions of glycosyltransferases in the Golgi apparatus¹¹⁻¹³. Cancer is often associated with dramatically higher expression of cell-surface mucins that bear truncated and more highly sialylated O-glycans^{9,14}. Through the engagement of Siglecs, the macrophage galactose receptor, and other immune-cell receptors, tumor-associated O-glycans have been implicated in the direct suppression of CD4⁺ T, CD8⁺ T, and NK cell activities^{7,15,16}, as well as the recruitment and education of tumor-associated macrophages¹⁷⁻¹⁹. Mucins and cancer-associated O-glycans also serve as scaffolds for multivalent galectins that can act as negative regulators, or checkpoints, of the immune cell functions²⁰⁻²². Immune suppression by mucins and mucin-type O-glycans is under active investigation as a mechanism of recalcitrance to cancer immunotherapies.

From a physical perspective, cell-surface mucins are semiflexible biopolymers that form a soft material coating on the tumor-cell surface^{23,24}. Although recent studies of immune modulation by mucins have focused on the biochemical activity of their O-glycans, it has been suggested that the dense grafting of mucin biopolymers on the tumor cell surface may form a steric barrier against immune-cell recognition and cytolytic activities²⁵⁻¹⁷. Direct validation of this possibility has remained elusive due to the challenge of disentangling the biochemical and material effects of specific changes in mucin-type glycosylation²⁷. The dense clustering of O-glycans along the mucin backbone both extends and rigidifies its macromolecular structure^{11,23,28}. Therefore, changes in the number, length, and sialylation (i.e. charge) of O-glycan chains are expected to alter the molecular properties of individual mucins and the ensemble material properties of the mucin-rich glycocalyx²³. Given the close, intimate contact that is required for target engagement by effector immune cells, the material thickness of the glycocalyx may be a major determinant of immune evasion. However, the investigation of how the physical dimensions of the glycocalyx change in response to altered states of glycosylation has been challenging due the nanometer-scale precision of measurement that is required to detect the structural changes²⁹.

Clinical interest in Natural killer (NK) cells for adoptive cell therapy has risen swiftly due to their ability to kill cells bearing markers associated with oncogenic transformation³⁰. NK cells have been equipped with chimeric antigen receptors (CAR-NK) to direct attacks against specific tumor antigens, with some CAR-NK therapeutics now in human clinical trials³⁰⁻³³. Compared to CAR-T cells, CAR-NK cells have some significant advantages that may include better safety and more potential for “off-the-shelf” manufacturing of allogeneic therapies. As is the case with other immune therapies, tumors can develop multiple mechanisms to resist attack by NK and CAR-NK cells. Notably, an inverse relationship between NK cell killing and the expression of cell-surface mucins on target cells has been reported²⁷. Truncation of mucin-type O-glycans increases NK cell-mediated antibody-dependent cellular-cytotoxicity, whereas elongation of the O-glycans has been reported to protect tumor cells^{26,27,34,35}. How such changes alter the material properties of the glycocalyx has been previously unknown, and it has been unclear if the effects on NK-cell resistance are caused by specific receptor interactions, changes in the structural attributes of the mucins, or some other consequence of altered glycosylation²⁷. There remains a need for new tools that can probe and measure the material properties of the glycocalyx to understand how these properties may contribute to immune evasion. Further, there is an ongoing and unmet need for improved approaches to overcoming the glycocalyx barrier that impedes anti-cancer immune responses. The present disclosure is pertinent to this need.

### BRIEF SUMMARY

The present disclosure provides modified eukaryotic cells comprising one or more plasma membrane-coupled glycocalyx-editing (GE) enzymes. In certain examples, the GE enzyme is a mucinase or a sialidase. Cells that include combinations of plasma membrane-coupled glycocalyx GEs are included in the disclosure. In certain examples the GE enzyme is coupled to the plasma membrane by a transmembrane domain that is a component of the GE enzyme, the GE enzyme is coupled to the plasma membrane by at least two agents that bind to one another, such as a leucine zipper. In one example, the sialidase is coupled to the plasma membrane by a transmembrane domain that is a component of the sialidase, and the mucinase is coupled to the plasma membrane by a leucine zipper that includes a transmembrane domain. In certain examples the modified cells are lymphocytes. In some examples the lymphocytes are T cells, natural killer (NK), natural killer T cells (NKTs), macrophages, or dendritic cells. In certain examples a modified T cell or a modified NK cell is also modified such that it expresses a chimeric antigen receptor (CAR).

Methods of making and using the modified cells are also included. The methods of making the modified cells comprise engineering the cells such that a GE enzyme is coupled to the plasma membrane by a transmembrane domain, a leucine zipper, or a combination thereof. The disclosure includes isolated populations of modified cells.

Methods of using the modified cell a comprise administering the modified cells to an individual in need thereof. In certain examples, the individual in need is an individual who has cancer, or is at risk of developing cancer, or cancer relapse.

### DESCRIPTION OF THE FIGURES

FIG. 1. Physical behavior of the mucin material barrier. (A) Doxycycline inducible system for controlled expression of Muc1-GFP with 42 tandem repeats. (B) Cartoon showing clonal expansion of epithelial cells expressing Muc1-GFP to isolate a stable clone, 1E7, with titratable mucin expression levels. (C) Flow cytometry analysis of the 1E7 clone showing titratable Muc1-GFP levels across the indicated concentrations of doxycycline inducer. (D) Fluorescence and widefield images of 1E7 cells induced at the indicated concentration of doxycycline (Scale bar, 10 μm). (E) Representative widefield fluorescence images and Ring-SAIM reconstructions of glycocalyx thickness in 1E7 cells at the indicated doxycycline induction levels and control epithelial cells that do not express Muc1-GFP (−Muc1-GFP). (F) Quantification of mean glycocalyx thickness in control cells (−Muc1-GFP) and 1E7 cells at the indicated induction levels. Results are the mean±s.d. of at least 18 cells. Statistical significance is given by ****P≤0.0001. (G) Log of the mean glycocalyx thickness in 1E7 cells versus the log of the mean GFP-nanobody signal, a proportionate measure of Muc surface density; best-fit line to a power-law model has slope=0.06 and R²=0.79. (H) Log of the mean glycocalyx thickness versus log of the mucin biopolymer length; best-fit line to a power-law model with slope=0.16 and R²=0.62. (I) Schematic summarizing the scaling behavior of the glycocalyx layer on mucin density, a, and chain size, L. (J) NK-92 cell-mediated lysis of control cells (−Muc1-GFP) and 1E7 target cells at the indicated doxycycline induction levels; results are shown for varying NK-92 to target cell ratios. Results are a representative dataset from 3 independent replicates. (Scale bar, 10 μm).

FIG. 2. Molecular determinants of the mucin material barrier. (A) Biosynthetic pathway and lectin probes for mucin-type core-I and core-II O-glycans. (B) Flow cytometry analysis of 1E7 cells and their glycoengineered progeny with knockout (KO) of C1GALT1 to truncate O-glycans, KO of GNE to ablate glycan sialylation, and overexpression (OE) of GCNT1 to enhance O-glycan extension; results for epithelial cells that do not express Muc1-GFP (−Muc1-GFP) are included as a control. (C) Western blot showing the relative size of Muc polymer in the 1E7 clone and glycoengineered progeny. (D) sWGA lectin blot after extended SDS-PAGE run time showing increased molecular weight of Muc1-GFP in GCNT1-overexpressing 1E7 cells compared to wild-type 1E7 cells. (E) Quantification of glycocalyx thickness in 1E7 cells, their glycoengineered progeny, and galectin-3 overexpressing cells (+Gal3 OE) at the indicated doxycycline induction levels; as indicated, some treatments include 10 μM recombinant galectin-1, 5 μM recombinant galectin-3, or 10 μM of the galectin inhibitor TD139. Results are the mean±s.d. of at least 13 cells. Statistical significance is given by ns—not significant, *P≤0.05, **P≤0.01, ***P≤0.001, ****P≤0.0001. Results are a representative dataset from 3 independent replicates.

FIG. 3. The mucin material barrier guards against NK cell-mediated cytotoxicity. (A) NK-92 cell-mediated cytotoxicity of 1E7 cells, their glycoengineered progeny (C1GALT1 KO, GNE KO, and GCNT1 OE), and 1E7 cells overexpressing galectin-3 (Gal-3 OE) at the indicated doxycycline induction level; NK cell to target cell ratio is 5:1. (B) NK-92 cell-mediated cytotoxicity is inversely proportional to the measured glycocalyx thickness with Pearson correlation coefficient, r=−0.94; dashed line indicates a linear fit to the data. (C) Cytotoxicity of NK cells against ZR-75-1 breast cancer cells sorted into three sub-populations with low, medium, and high levels of endogenous Muc1 expression; NK cell to target cell ratio is 5:1. Data presented as the mean and ±s.d. of three independent experiments. Statistical significance is given by ns—not significant, *P≤0.05, **P≤0.01, ***P≤0.001, ****P≤0.0001. Results are a representative dataset from 3 independent replicates.

FIG. 4. Tethering StcE mucinase to the NK cell surface enhances their cytolytic efficiency. (A) Cartoon showing preparation of StcE-NK through tethering of StcE mucinase to the NK-92 cell surface by its X409 lectin domain. (B) Flow cytometry analysis of StcE surface levels on NK cells incubated with wild-type StcE and StcE with deletion of the X409 lectin domain (StcE ΔX409) at the indicated concentration; StcE surface level is probed with anti His-tag antibody. (C) Cytotoxicity of control NK-92 cells and NK-92 cells treated with wild-type StcE (StcE-NK), catalytically dead StcE E447D (StcE-E447D NK), and StcE with deletion of the X409 lectin domain (StcE-ΔX409 NK) against 1E7 cells at the indicated doxycycline induction level of Muc1-GFP expression; StcE-NK cells, StcE E447D-NK cells, and StcE ΔX409-NK were prepared through incubation with 100 nM StcE, StcE E447D, or StcE ΔX409; NK cell to target cell ratio is 10:1. (D) Cartoon showing a modular strategy for coupling glycocalyx editing (GE) enzymes to the NK surface using leucine zippers; to create StcE-Zip NK cells, recombinant StcE with the X409 domain swapped for the EE leucine zipper (StcE-EE) is coupled to a membrane-anchored, cognate zipper (RR) stably expressed on the NK cell surface. (E) Mean StcE surface level on wild-type NK-92 cells and those expressing surface RR treated with StcE-EE Zip at the indicated concentration for 1 hour; StcE surface level is probed with anti His-tag antibody and the fluorescence signal is normalized to the mean signal for untreated cells. (F) Cytotoxicity of StcE-Zip NK cells, prepared with the indicated concentration of StcE-EE, against 1E7 cells; Muc1-GFP induction is at the indicated doxycycline concentration; NK cell to target cell ratio is 5:1. (G, H) Representative Muc1-GFP and brightfield images of 1E7 cells interacting with NK-92 (G) and StcE-NK (H) cells; lower images show zoomed-in regions of the NK-1E7 interface. (Scale bar, 10 μm). (I) Plot of the mean Muc1-GFP intensity (dark lines) and standard deviations (lighter bounding areas) along line profiles across the contact interface between 1E7 cell and NK cells (n=6) or StcE-NK cells (n=10); the fluorescence intensity is normalized to the mean fluorescence intensity outside of the contact interface; red dotted arrows in G and H show representative profile lines. (J, K) Cytotoxicity of NK cells and 10 nM StcE-NK cells against unsorted ZR-75-1 (J) and T47D (K) breast cancer cells that endogenously express Muc1; NK cell to target cell ratio is 5:1. Data presented as the mean and ±s.d. of three independent experiments. Statistical significance is given by *P≤0.05, **P≤0.01, ***P≤0.001, ****P≤0.0001.

FIG. 5. The glycocalyx is a barrier against engineered immune cell therapies. (A) Cartoon showing construction of NK-92 cells equipped with HER2 chimeric antigen receptor (CAR-NK) and CAR-NK cells additionally equipped with StcE mucinase (StcE CAR-NK). (B) Western blot analysis of wild-type (control) and HER2 overexpressing 1E7 cells. (C) Cytotoxicity of NK, StcE-NK, CAR-NK, and StcE CAR-NK against HER2-overexpressed 1E7 clone at the indicated concentration of doxycycline; NK cell to target cell ratio is 5:1. (D) Quantification of mean glycocalyx thickness in the indicated cells before and after treatment of StcE; 1E7 cells induced 1,000 ng/ml doxycycline. (E) Percent reduction of glycocalyx thickness by StcE treatment in the indicated cells from (D). (F, G, H) Cytotoxicity of NK, StcE-NK, CAR-NK, and StcE CAR-NK against SKOV3 (F), SKBR3 (G) and OVCAR3 (H). NK cell to target cell ratio is 10:1. Statistical significance is given by *P≤0.05, **P≤0.01, ***P≤0.001, ****P≤0.0001.

FIG. 6. Measurement of the glycocalyx material thickness with Ring Scanning Angle Interference Microscopy (Ring-SAIM). (A) Schematic illustration of Ring-SAIM; interference of direct and reflected laser light off a silicon mirror create standing waves of excitation light that depend on the laser incidence angle, θ, and are used to probe the vertical position of fluorescence emitters. (B) Optical configuration for Ring-SAIM showing azimuthally scanned excitation light at varying θ; AOTF, Acousto-optic tunable filter; Polarizer, m=1 vortex half-wave retarder. (C) Muc1-GFP constructs with biopolymer domains comprised of 0, 10, 21, and 42 tandem repeats (TRs); GFP-nanobody specifically labels the cell-surface fraction of Muc1-GFP. (D) Western blot analysis of Muc1-GFP constructs stably expressed in MCF10A epithelial cells; arrows indicate fully glycosylated Muc with 10, 21, or 42 TRs. (E) Examples of the pixelwise Ring-SAIM interferograms for Muc1-GFP constructs labelled with AF647-GFP nanobody. (F) Flow cytometry analysis of cell-surface Muc1-GFP probed with Alexa Fluor 647 (AF647) conjugated GFP nanobody. (G) Representative widefield image and glycocalyx thickness map of live epithelial cells expressing the indicated Muc1 constructs labeled with AF647 GFP nanobody (scale bars, 10 μm). (H) Quantification of mean glycocalyx thickness in cells expressing the Muc constructs. Results are the mean±s.d. of at least 19 cells. Statistical significance is given by *P≤0.05, ***P≤0.001, ****P≤0.0001. Results are a representative dataset from 3 independent replicates.

FIG. 7. Analysis of NK-cell mediated killing in additional Muc1-GFP expressing clones. (A,B) Representative confocal and bright-field images of two clonally expanded MCF10A cell lines expressing Muc1-GFP with 42 tandem repeats under the control of a tetracycline inducible promoter; the doxycycline induction levels are indicated. (Scale bar, 10 μm). (C) Cytotoxicity of NK-92 cells against three clonally expanded cell populations and the parental polyclonal cell line. The doxycycline induction levels are indicated. NK cell to target cell ratio is 5:1. Statistical significance is given by ns—not significant, *P≤0.05, ***P≤0.001, ****P≤0.0001. Results are a representative dataset from 3 independent replicates.

FIG. 8. Validation of glycoengineered cell lines. (A) Agarose gel electrophoresis of PCR-amplified and EcoRI-digested segment C1GALT1 showing successful homozygous knockout (KO) in 1E7 cells (See 2A7 sub-clone of 1E7). For the KO, a stop codon and an EcoRI restriction site for screening were inserted in the C1GALT1 gene via CRISPR/Cas9 and homology directed repair. (B) Western blot analysis confirming UDP-N-acetylglucosamine 2-epimerase (GNE) KO in 1E7 cells. (C) Fluorescence images of GCNT1 in 1E7 clone and GCNT1 overexpression in the 1E7 clone induced at 1,000 ng/ml of doxycycline (Scale bar, 10 μm).

FIG. 9. Confirmation of galectin-1 and galectin-3 interactions with mucin-type 0-glycans (A) Quantification of recombinant galectin-1 (Gal-1) binding to Muc1-GFP expressing 1E7 cells and their glycoengineered progeny with knockout of C1GALT1 to ablate Core-I extension of O-glycans and overexpression (OE) of GCNT1 to increase Core-II extension; induction of Muc1-GFP is at 1,000 ng/ml of doxycycline; data normalized to the Muc signal intensity and presented as the mean and ±s.d. of 20 cells (B) Quantification of recombinant galectin-3 (Gal-3) binding to the 1E7 cells and their glycoengineered progeny; data normalized to the Muc signal intensity and presented as the mean and ±s.d. of 20 cells. (C) Same as in b with 5,000 U/mL PNGase F to selectively removes N-glycans prior to analysis. (D) Quantification of endogenous galectin-3 (left) and Muc (right) on cell surface before and after 200 nM of StcE mucinase treatment, confirming that galectin-3 binds specifically to Muc1. (E) Representative confocal images of permeabilized 1E7 clone and Gal-3 overexpression in the 1E7 clone induced at 1,000 ng/ml of doxycycline (Scale bar, 10 μm). (F) Representative confocal images of endogenous galectin-3 on the cell membrane in presence and absence of TD139 (Scale bars, 10 μm). (G) Quantification of galectin-3 (Gal3) binding to 1E7 clone surface induced 1,000 ng/ml of dox with TD139; data normalized to the Muc signal intensity and presented as the mean and ±s.d. of 10 cells. Statistical significance is given by **P≤0.01, ****P≤0.0001.

FIG. 10. Galectin-1 does not significantly affect the mobility and mobile fraction of cell surface Muc1. (A) FRAP analysis was performed on 1E7 clone (red), 1E7 GCNT1 OE (green), 1E7 GCNT1 OE with 10 μM recombinant human galectin-1 (blue). n=11, 10, 9 respectively (B,C) Quantitative analysis of mobile fraction (B) and half recovery time (C). (D) Representative confocal images of each condition at pre-bleach, post-bleach, and recovery. (Scale bar, 10 μm). Statistical significance is given by ns—not significant. Results are a representative dataset from 3 independent replicates.

FIG. 11. StcE mucinase effectively remove cell-surface mucin on 1E7 clone and NK cell. (A) Representative confocal images of Muc1-GFP expressing 1E7 cells with and without treatment with 100 nM StcE, StcE ΔX409, or StcE-EE Zip; Alexa Fluor 647 conjugated to GFP nanobody is used to probe cell-surface Muc1-GFP constructs. (Scale bar, 10 μm). (B) Quantification of mean fluorescence intensity of Muc1-GFP and cell-surface Muc1-GFP probed with GFP nanobody in 1E7 cells treated with the indicated concentration of StcE mucinase; results are shown for induction of Muc1-GFP at 100 ng/ml and 1000 ng/ml doxycycline. (C) Cytotoxicity of NK-92 cells against 1E7 cells treated with control buffer, 100 nM StcE mucinase, or 5,000 U/mL PNGase F for 1 hour; Muc1-GFP induction is at the indicated doxycycline concentration (1,100, and 1,000 ng/ml); NK cell to target cell ratio is 5:1. (D) Flow cytometry analysis of mucin levels on NK-92 cells following incubation with control buffer or 100 nM recombinant StcE or StcE ΔX409; StcE surface level after incubation probed with anti His-tag antibody and cell-surface mucin level probed with catalytically dead StcE E447D conjugated to Cy5. Statistical significance is given by ns—not significant, *P≤0.05, **P≤0.01, ***P≤0.001, ****P≤0.0001. Results are a representative dataset from 3 independent replicates.

FIG. 12. Modular strategy for Enzyme-Zip NK cell (A) Cartoon showing an updated modular strategy for coupling glycocalyx-editing (GE) enzymes to the NK cell surface using leucine zipper; to create GE-Zip NK cells, recombinant GE enzyme with the EE leucine zipper (Enzyme-EE) is coupled to a membrane-anchored, cognate zipper (RR) stably expressed on the NK cell surface. For StcE, X409 lectin domain of StcE was swapped to the EE leucine zipper. RR leucine zipper is linked with extracellular ALFA-Tag, PDGFRP transmembrane domain and intracellular sfGFP. (B) Flow cytometry analysis of expression level of cell-surface RR leucine zipper on NK-RR Zip cells with sfGFP and anti-ALFA-tag Alexa Flour 647. (C) Cytotoxicity of wild-type NK cells and StcE-Zip NK cells, prepared with the indicated concentration of StcE-EE, against MCF10A cells expressing titratable Muc dCT construct (B2 clone); Muc induction is at the indicated doxycycline concentration; NK cell to target cell ratio is 5:1. (D) Flow cytometry analysis of surface levels of EE Zip-tagged mucinases on NK cells incubated with each mucinaeses at the indicated concentration; surface level of mucinases is probed with anti His-tag antibody. (E) Dose-response plot of normalized mean intensity for (D); Kd values determined by sigmoidal fitting.

FIG. 13. Combinatorial approach for GE-NK. (A) Cartoon showing a combinatorial enzyme approach for mucinase and sialidase. (B) Flow cytometry analysis of total sialic acid on cell-surface of 1E7 cells incubated with Sialidase at the indicated concentration by using Periodate oxidation and Aniline catalyzed oxime Ligation (PAL) with Amminooxy-CF680; 1E7 GNE KO used as a negative control. (C) Normalized cytotoxicity of NK-Zip and Sialidase+AM0627-NK (90 nM AM0627; 10 nM Sialidase) against T47D, Capan2, and ZR-75-1; NK cell to target cell ratio is 5:1. (D) Normalized cytotoxicity of NK-Zip, Sialidase-NK, AM0627-NK, Sialidase+AM0627 (1:1)-NK, BT4244-NK, Sialidase+BT4244 (1:1)-NK against 1E7 clone with 1,000 ng/ml of doxycycline, OVCAR3, SKBR3, and SKOV3; NK cell to target cell ratio is 5:1.

FIG. 14. Genetic expression of sialidase-transmembrane fusion proteins in NK cells. (A) Cartoon showing an approach for the genetic expression of sialidase on the NK cell surface; the sialidase is fused to the PDGFRβ transmembrane domain (TM) with a cytoplasmic sfGFP reporter and expressed under the control of a doxycycline inducible promoter. (B) Fluorescence images of NK cells expressing the sialidase-TM-sfGFP construct following induction of expression at the indicated concentration of doxycycline (Scale bar, 10 μm). (C) The cytotoxicity of NK-Zip (a control NK cell line that does not express sialidase) and Sialidase-TM-GFP expressing NK cells against target cells at an NK:target ratio of 5:1.

FIG. 15. Mucin barrier against other immune cells. (A) Representative flow plots depicting surface CD3 and CD56 levels of NK-92 cell line and primary NK cell. (B, C, D) Flow cytometry analysis of cell-surface CD56 (B), Siglec-7 (C), Siglec-9 (D) levels of NK-92 cell line and primary NK cells. (E) Quantification of Siglec-7 expression level on NK cells from (C). (F, G) Cytotoxicity of NK-92 and primary NK cells against 1E7 and 1E7 GNE KO; Muc1 induction is at the indicated doxycycline concentration. (H) Cytotoxicity of Horse PBMCs against 1E7; Muc induction is at the indicated doxycycline concentration. (I) Flow cytometry analysis of cell surface CD19 expression level on 1E7 and CD19 overexpressed 1E7 at varying doxycycline concentrations. (J) Cytotoxicity of CD19 CAR NK against 1E7 and CD19 overexpressed 1E7; Muc induction is at the indicated doxycycline concentration. NK cell to target cell ratio is 5:1. Data presented as the mean and ±s.d. of three independent experiments. Statistical significance is given by ns—not significant, *P≤0.05, **P≤0.01, ***P≤0.001, ****P≤0.0001.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification includes every narrower numerical range that falls within such broader numerical range, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value encompasses variations of +/−10%, +/−5%, or +/−1%.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included.

The disclosure includes all polynucleotide and all amino acid sequences that are identified herein by way of a database entry. Such sequences are incorporated herein as they exist in the database on the effective filing date of this application or patent.

The disclosure provides modified cells, methods of using the modified cells, methods of making the modified cells, and the modified cells themselves. In general, the cells are modified such they include a plasma membrane-coupled glycocalyx-editing (GE) enzyme. The GE enzyme may be any GE enzyme that is not expressed by the cell without modification, and/or any GE enzyme that is not plasma membrane-coupled absent a described modification. “Plasma-membrane coupled” as used herein means the described protein is connected to the plasma membrane directly or indirectly such that the described protein can modify the glycocalyx of other cells. As such, at least an enzymatically active segment of the described protein is positioned externally relative to the plasma membrane. In one embodiment, the GE enzyme is coupled to the plasma membrane by a transmembrane domain that is a component of the described protein. In another embodiment, a first protein that comprises a transmembrane domain is modified such that that it non-covalently associates with a second protein that is cognate to the first protein. For instance, first and second leucine zipper proteins can be used. One of the leucine zipper proteins comprises a transmembrane domain and is expressed by the modified cell. The other leucine zipper protein is added to the GE enzyme. By combining the GE enzyme comprising a leucine zipper protein that is cognate to the leucine zipper comprised by the modified cell the modified GE enzyme will be coupled to the plasma membrane through the leucine zipper protein pairing. Thus, in an embodiment, a described modified cell is engineered to express a first leucine zipper protein that comprises a transmembrane domain, and a modified GE enzyme that comprises a cognate leucine zipper that is provided to the cells such that the modified GE enzyme is coupled to the plasma membrane by the assembled leucine zipper. In one embodiment, a domain of the GE enzyme that is not a catalytic domain is substituted with a leucine zipper protein to thereby produce an enzymatically active fusion protein comprising a GE catalytic domain and a leucine zipper protein.

In alternative approaches, the described leucine zipper approach may be substituted with other agents. Such agents include but are not necessarily first and second binding agents, wherein the first and second binding agents that are not leucine zipper proteins bind to one another. For instance, an avidin-biotin binding interaction could be used. In embodiments, non-leucine zipper protein tags may be used. In embodiments, one or more epitope tag formats may be used in combination with an antibody or antigen binding segment thereof that specifically binds to the epitope tag.

Representative and non-limiting examples formats that may be used to couple the GE enzyme to the plasma membrane include SpyTag, ALFA-Tag, SNAP-tag, CLIP tag, maltose binding protein, Fc-tag, and Halo-Tag approaches. In other embodiments, the GE enzyme may be coupled to a plasma membrane protein through click chemistry approaches, such as azide-alkyne cycloaddition.

Any suitable GE enzyme can be used. In embodiments, the GE enzyme is a mucinase or a sialidase. In non-limiting embodiments the GE enzyme component of a described protein comprises or consists of the entire protein, or comprises or consists of a catalytic domain. As such, certain amino acids of the GE enzyme may be excluded, changed, or substituted. In embodiments, a signal peptide is excluded from the GE enzyme.

In non-limiting embodiments, the mucinase component comprises or consists of all or a catalytically active segment of secreted protease of C1 esterase inhibitor (StcE), a bacterial protease from Escherichia coli. The amino acid sequence of StcE is known and can be accessed via UniProtKB/Swiss-Prot: O82882.2. A representative full length StcE amino acid sequence is:

(SEQ ID NO: 1)

| | |
|---|---|
| 1 | mntkmnerwr tpmklkylsc tilaplaigv fsataadnns |
| | aiyfntsqpi ndlqgslaae |
| 61 | vkfaqsqilp ahpkegdsqp hltslrksll lvrpvkaddk |
| | tpvqveardd nnkilgtltl |
| 121 | yppsslpdti yhldgvpegg idftphngtk kiintvaevn |
| | klsdasgssi hshltnnalv |
| 181 | eihtangrwv rdiylpqgpd legkmvrfvs sagysstvfy |
| | gdrkvtlsvg ntllfkyvng |
| 241 | qwfrsgelen nrityaqhiw saelpahwiv pglnlvikqg |
| | nlsgrlndik igapgelllh |
| 301 | tidigmlttp rdrfdfakdk eahreyfqti pvsrmivnny |
| | aplhlkevml ptgelltdmd |
| 361 | pgnggwhsgt mrqrigkelv shgidnanyg lnstaglgen |
| | shpyvvaqla ahnsrgnyan |
| 421 | giqvhggsgg ggivtldstl gnefshevgh nyglghyvdg |
| | fkgsvhrsae nnnstwgwdg |
| 481 | dkkrfipnfy psqtnekscl nnqcqepfdg hkfgfdamag |
| | gspfsaanrf tmytpnssai |
| 541 | iqrffenkav fdsrsstgfs kwnadtqeme pyehtidrae |
| | qitasvnels eskmaelmae |
| 601 | yavvkvhmwn gnwtrniyip tasadnrgsi ltinheagyn |
| | sylfingdek vvsqgykksf |
| 661 | vsdgqfwker dvvdtreark peqfgvpvtt lvgyydpegt |
| | lssyiypamy gaygftysdd |
| 721 | sqnlsdndcq lqvdtkegql rfrlanhran ntvmnkfhin |
| | vptesqptqa tlvonnkild |
| 781 | tksltpapeg ltytvngqal pakenegciv svnsgkrycl |
| | pvgqrsgysl pdwivgqevy |
| 841 | vdsgakakvl lsdwdnlsyn rigefvgnvn padmkkvkaw |
| | ngqyldfskp rsmrvvyk. |

The italicized sequence is a signal peptide sequence. The bold sequence is the X409 sequence.

In an embodiment, the first 35 amino acids of the StcE protein, which constitute a signal sequence, are deleted. In an embodiment, the X409 lectin domain of StcE is replaced with a leucine zipper to create a fusion between the StcE catalytic domain (amino acids 36-796 of StcE as shown in SEQ ID NO:1) and the leucine zipper. This fusion is then produced recombinantly and tethered to the immune cell surface through coupling with the cognate leucine zipper expressed genetically in the immune cells as a transmembrane fusion protein.

In another embodiment, the mucinase may be BT4244 from Bacteroides thetaiotaomicron or M60-like protease AM0627 from Akkermansia muciniphila.

The amino acid sequence of BT4244 is known and is available under GenBank sequence WP_012419679.1. A representative full length sequence of BT4244 is:

(SEQ ID NO: 2)

| | |
|---|---|
| 1 | mnrilpllsl pllslaavfa antpehignd lklfkdssct |
| | slkpdvknts afqsdamkel |
| 61 | atkilaghyk pdylyaeyra lpsprqtgkn lrigdgfsky |
| | dnmtgvylek grhvvlvgkt |
| 121 | egqeislllp nlmrkpaegv qptkdpngwg lhkkqiplke |
| | giniidvetp anayisyfte |
| 181 | dagkapkipv hfvtgkangy fdttrgdtnk dwvrlldqav |
| | spimdargky iqvaypvefl |
| 241 | kkftkdrgte linaydklig iqyqlmgldk ygkipenrvl |
| | arvnfnyymf rdgdgvaylg |
| 301 | ndgtmrmvtd penvlkgdac wgfshevghv mqmrpmtwgg |
| | mtevsnnifs lqaaaktgne |
| 361 | srlkrqgsyd karkeiiege iaylqskdvf nklvplwqlh |
| | lyftknghpd fypdvmeylr |
| 421 | nnagnyggnd tvkyqfefvk accdvtktdl tdffekwgff |
| | kpgkfhigdy aqydfnvtpe |
| 481 | mveetkkwia gkgypkpetd itelse. |

In an embodiment, the first 10 amino acids, which constitute a signal sequence, can be deleted from the BT4244 protein.

The amino acid sequence of AM0627 and is available from GenBank sequence WP_008764444.1. A representative full length sequence of AM0627 is:

(SEQ ID NO: 3)

| | |
|---|---|
| 1 | mtikrfitnl lalftlftvs lackdteksi inssfsisee |
| | yliqnldkss tsvqipints |
| 61 | melaqwsvsy eanwlqcskq ktaaegtflr itvnentget |
| | krtanikvts ttatytitvn |
| 121 | qyakgevive gdikvtptgg kasehqegqd ientydgkfs |
| | tdgaapfhtp wgqsakfpvt |
| 181 | leyyfkgdte idyliyytrs gngnfgkvkv ytttnpdrsd |
| | ytlqgeydfk eqnapskvsf |
| 241 | segikatgik fevlsglgdf vscdemefyk tntdktldkq |
| | lltvftditc teiknnvtne |
| 301 | qiqalpdyfv riaeavrdnt ydkwekefri rsyepysnia |
| | ewadklmtkk ysdldnptgi |
| 361 | svkagddiiv lvgdtygqni smqciwetgt eykqtassgd |
| | vymlnpgvnk ltmkgegqlf |
| 421 | vmynteltsn takpikihip lgsgtvngff dlkehktdek |
| | yaellkksth kyfcirgeki |
| 481 | mfyfhrnkll eyvpnnilsa ihlwdnivgw qqelmgiddv |
| | rpsqvnnhlf aispegsymw |
| 541 | asdyqigfvy tylgnilled nvmaaednaw gpaheighvh |
| | qaainwasst essnnlfsnf |
| 601 | iiyklgkyks rgnglgsvat aryangqawy nmgdathqne |
| | dtethmrmnw qlwiyyhrce |
| 661 | yktdfwqtlf klmrevnmte gedpgkkqle fakmaskaan |
| | qnltdffemw gffepvntti |
| 721 | eqygtykyyv sdamireake ymaqfpapkh afqyiedrkk |
| | sefpsndyry savgdvgyyt |
| 781 | qfkenqkitk aitaelagrk vsiqngdeav afelrenden |
| | gkllyfstft tfeipssilm |
| 841 | vnaklyavqa dgkrill. |

In embodiments, the first 34 amino acids, which constitute a signal sequence, can be deleted from the BT4244 protein.

In embodiments, the GE enzyme comprises or consists of all or a segment of a sialidase. Suitable sialidases are known in the art. In one embodiment, the sialidase comprises a neuraminidase. In one embodiment, the sialidase comprises a sequence that is available under GenBank accession number AAA27168.1. A representative amino acid sequence of this protein is:

(SEQ ID NO: 4)

| | |
|---|---|
| 1 | mtveksvvfk aegehftdqk gntivgsgsg gttkyfripa |
| | mcttskgtiv vfadarhnta |
| 61 | sdqsfidtaa arstdggktw nkkiaiyndr vnsklsrvmd |
| | ptcivaniqg retilvmvgk |
| 121 | wnnndktwga yrdkapdtdw dlvlykstdd gvtfskvetn |
| | ihdivtkngt isamlggvgs |
| 181 | glqlndgklv fpvqmvrtkn ittvlntsfi ystdgitwsl |
| | psgycegfgs enniiefnas |
| 241 | lvnnirnsgl rrsfetkdfg ktwtefppmd kkvdnrnhgv |
| | qgstitipsg nklvaahssa |
| 301 | qnknndytrs dislyahnly sgevklidaf ypkvgnasga |
| | gysclsyrkn vdketlyvvy |
| 361 | eangsiefqd lsrhlpviks yn |

As discussed above, in certain embodiments, the GE enzyme component may be tethered to the plasma membrane using a leucine zipper approach. The amino acid sequences of leucine zipper protein pairs are known in the art. In embodiments, a first leucine zipper protein may comprises or consist of the sequence MDPDLEIEAAFLERENTALETRVAELRQRVQRLRNRVSQYRTRYGPLGGGK (SEQ ID NO:5). In non-limiting embodiments this sequence is tethered to the GE enzyme. In embodiments, a second leucine zipper protein may comprise or consist of the sequence MDPDLEIRAAFLRQRNTALRTEVAELEQEVQRLENEVSQYETRYGPLGGGK (SEQ ID NO:6). In embodiments, this sequence may be expressed by a modified cell, and as such, it may also comprise a transmembrane domain.

In certain examples, a described protein has a segment joined to another segment using linking amino acids, i.e., a linker. In certain examples, a described protein may comprise linking amino acids that connect a first component to a second component. Suitable amino acid linkers may be mainly composed of relatively small, neutral amino acids, such as glycine, serine, and alanine, and can include multiple copies of a sequence enriched in glycine and serine. In specific and non-limiting embodiments, the linker comprises 3, 4, 5, 6, 7,8,9, 10, 11, 12, 13, 14, 15, 16, 17, 18, 19, 20, 21, 22, 23, 24, 25, 26, 27, 28, 29, 30, 31, 32, 33, 34, 35, or more amino acids. In embodiments, a GE enzyme is connected to a leucine zipper using a 35 amino acid linker, a non-limiting example comprises

| (SEQ ID NO: 7) | |
|---|---|
| | GGGGSGGGGSGGGGSGGGGSGGGGSGGGGSGGGGS. |

In non-limiting embodiments, modified cells of the disclosure may display only one plasma membrane coupled GE. In embodiments, modified cells of the disclosure may display more than one plasma membrane coupled GE. In a non-limiting embodiment, a sialidase is coupled to the plasma membrane by a transmembrane domain that is a component of the sialidase, such as in a fusion protein. In a non-limiting embodiment, a mucinase is coupled to the plasma membrane by a leucine zipper. The disclosure also includes a plasma-membrane coupled mucinase that comprises a transmembrane domain, a sialidase that is coupled to the plasma membrane by a leucine zipper, and combinations of the described modifications. Single cells that are modified to have both a plasma-membrane coupled sialidase that comprises a transmembrane domain and to have a mucinase that is coupled to the plasma membrane by a leucine zipper are encompassed by the disclosure.

In embodiments, any suitable plasma-membrane anchor sequence can be used. In embodiments, the transmembrane segment comprises about 20 residues of suitable hydrophobicity so that an α-helix can span the apolar core of a plasma membrane bilayer. Suitable transmembrane domains include, but are not limited to: a member of the tumor necrosis factor receptor superfamily, CD30, platelet derived growth factor receptor (PDGFR, e.g. amino acids 514-562 of human PDGFR; Chestnut et al., 1996, J Immunological Methods, 193:17-27; also see Gronwald et al., 1988, PNAS, 85:3435-3439); nerve growth factor receptor, Murine B7-1 (Freeman et al., 1991, J Exp Med 174:625-631), asialoglycoprotein receptor H1 subunit (ASGPR; Speiss et al. 1985 J Biol Chem 260:1979-1982), CD27, CD40, CD120a, CD120b, CD80 (B7) (Freeman et al., 1989, J Immunol, 143:2714-2272) lymphotoxin beta receptor, galactosyltransferase (e.g., GenBank accession number AF155582), sialyltransferase (E.G. GenBank accession number NM-003032), aspartyl transferase 1 (Asp1; e.g. GenBank accession number AF200342), aspartyl transferase 2 (Asp2; e.g. GenBank accession number NM-012104), syntaxin 6 (e.g. GenBank accession number NM-005819), ubiquitin, dopamine receptor, insulin B chain, acetylglucosaminyl transferase (e.g. GenBank accession number NM-002406), APP (e.g. GenBank accession number A33292), a G-protein coupled receptor, thrombomodulin (Suzuki et al., 1987, EMBO J, 6:1891-1897) and TRAIL receptor. The sequence of the plasma-membrane anchor used in the Examples of this disclosure is

(SEQ ID NO: 8)
AVGQDTQEVIVVPHSLPFKVVVISAILALVVLTIISLIILIMLWQKKPR.

In embodiments, the modified cells comprise any type of eukaryotic cells. In embodiments, the modified cells are mammalian cells. In embodiments, the modified cells are human cells. In embodiments, the cells are leukocytes. In embodiments, the cells are lymphocytes. In embodiments, the modified cells are immune cells. In embodiments, the modified cells are CD4+ T cells, CD8+ T cells (i.e, cytotoxic T cells), Natural Killer T cells (NKTs), i.e., a subset of T-cells that express T-cell receptor αβ chains as well as NK cell markers, or γδ T cells, or cells that are progenitors of T cells, such as hematopoietic stem cells or other lymphoid progenitor cells, such as immature thymocytes (double-negative CD4−CD8−) cells, or double-positive thymocytes (CD4+CD8+). In embodiments, the modified cells are NK cells, including but not limited to the CD56^{high}, CD56^{low}, CD56^{high}/CD16^{high}, CD56^{high}/CD16^{low}, and CD56^{low}/CD16^{high} subsets of NK cells. Such markers and expression levels will be understood by those skilled in the art, and can be used by the skilled artisan to differentiate subsets of NK cells. In embodiments, NK cells modified according to the disclosure are CD3−/CD56+ lymphocytes. In embodiments, the modified cells are macrophages or dendritic cells. In embodiments, the modified cells are primary cells that are obtained from an autologous or allogenic donor. In embodiments, the modified cells are primary cells that are obtained from an autologous or allogenic donor. In the embodiments, the modified cells are immortalized cells grown in culture. In the embodiments, the modified cells are pluripotent stem cells or induced pluripotent stem cells (iPSC). In the embodiments, the modified cells are derived from pluripotent stem cells or iPSC cells.

In embodiments, the modified cells are NK cells or T cells that have also engineered to express a chimeric antigen receptor (CAR). The type of CAR is not particularly limited, but in general is configured to enhance an anti-cancer immune response. Representative CARs are discussed in the Examples.

In one embodiment, a biological sample from an individual is analyzed to determine the presence of cancer cells. The cancer cells if present can be analyzed to characterize the glycocalyx exhibited by the cancer cells. Based at least in part on the characterization of the glycocalyx, a suitable single or combination of GE enzymes can be selected and used to produce and use modified cells according to this disclosure for therapy of the individual.

The disclosure includes isolated modified cells as described herein, and methods of making the modified cells. Compositions used in making the modified cells are also includes within the scope of the disclosure. As such, isolated modified GE enzymes, and expression vectors encoding them, are encompassed by the disclosure.

In embodiments, a genome of one or more cells as described herein may be modified to express any described protein, such as a GE enzyme, or a leucine zipper component, either of which may be modified to include a transmembrane domain. This approach can be performed using any designer nuclease. In embodiments, the nuclease is a RNA-guided CRISPR nuclease. A variety of suitable CRISPR nucleases (e.g., Cas nucleases) are known in the art. In specific and non-limiting embodiments, the Cas comprises a Cas9, such as Streptococcus pyogenes (SpCas9). Derivatives of Cas9 are known in the art and may also be used. Such derivatives may be, for example, smaller enzymes than Cas9, and/or have different proto adjacent motif (PAM) requirements. In a non-limiting embodiments, the Cas enzyme may be Cas12a, also known as Cpf1, or SpCas9-HF1, or HypaCas9. In embodiments, a protein may be administered directly to the cells. For example, a Cas protein and suitable guide RNA(s) may be administered to the cells as ribonucleoproteins (RNPs) using any suitable technique. Alternatively, the Cas protein may be introduced into the cells separately from the guide RNAs. In embodiments, a viral expression vector may be used to introduce sequences encoding one or more of the described proteins. Expression of any of the described proteins can be constitutive or induced. In embodiments, one or more of the described proteins are stably expressed within cells from an introduced genetic element, or from an extra-chromosomal element.

In embodiments, an effective amount of modified cells is administered to an individual in need thereof. In embodiments, an effective amount is an amount of cells that reduces one or more signs or symptoms of a disease and/or reduces the severity of the disease. An effective amount may also inhibit or prevent the onset of a disease or a disease relapse. A precise cellular dosage can be selected by the individual physician in view of the patient to be treated. Dosage and administration can be adjusted to provide sufficient levels of the described modified to maintain the desired effect. Additional factors that may be taken into account include the severity and type of the disease state, age, weight, and gender of the patient, desired duration of treatment, method of administration, time and frequency of administration, drug combination(s), reaction sensitivities, and/or tolerance/response to therapy. The described modified cells and pharmaceutical compositions comprising the modified cells can be administered to an individual in need thereof using any suitable route, examples of which include intravenous, intramuscular, intraperitoneal, intracerobrospinal, and subcutaneous routes. The modified cells may be introduced as a single administration or as multiple administrations or may be introduced in a continuous manner over a period of time.

In embodiments, the individual in need of modified cells of this disclosure has been diagnosed with or is suspected of having cancer. In embodiments, the cancer is a solid tumor or a hematologic malignancy. In embodiments, the cancer is renal cell carcinoma, breast cancer, prostate cancer, pancreatic cancer, lung cancer, liver cancer, ovarian cancer, cervical cancer, colon cancer, esophageal cancer, glioma, glioblastoma or another brain cancer, stomach cancer, bladder cancer, testicular cancer, head and neck cancer, melanoma or another skin cancer, any sarcoma, including but not limited to fibrosarcoma, angiosarcoma, osteosarcoma, and rhabdomyosarcoma, and any blood cancer, including all types of leukemia, lymphoma, and myeloma. In embodiments, the individual in need thereof has cancer cells that exhibit an altered glycocalyx, relative to the glycocalyx of non-cancer cells. In embodiments, cancer cells from the individual overexpress one or more mucins, relative to expression of mucins by non-cancer cells. In embodiments, cancer cells from the individual express one or more different mucines, relative to non-cancer cells. In embodiments, overexpressed mucins by cancer cells comprise galectin-3, GCNT1, or a combination thereof. In embodiments, cancer cells from the individual have an increased thickness of their glycocalyx, relative to the thickness of the glycocalyx of non-cancer cells. In embodiments, cancer cells from the individual have increased mucin surface density, relative to the surface density of non-cancer cells. In embodiments, cancer cells from the individual have increased sialic acid surface density, relative to the surface density of non-cancer cells. In embodiments, the described modified cells may be used prophylactically for any of the described types of cancer.

In embodiments, the described modified cells exhibit an improved activity relative to a control, such as control cells. In embodiments, administering described modified cells to an individual in need thereof exhibits an improved activity relative to a control. In embodiments, the control comprises unmodified cells. In embodiments, the control comprises cells that are modified to display only one GE enzyme, which is compared to modified cells that have more than one plasma-membrane coupled GE enzyme, such as at least two, or only two GE enzymes. In embodiments, the control comprises a different type of cell than the modified cell. In an embodiment, coupling of both a sialidase and a mucinase improves cell mediated cytotoxicity relative to unmodified cells. In an embodiment, the modified cells are NK cells, and exhibit improved cytotoxicity of cancer cells relative to unmodified NK cells. In an embodiment, the improved cytotoxicity comprises a 1-20 fold enhancement in cytotoxicity relative to cytotoxicity of non-modified NK cells. In embodiments, the modified NK cells exhibit at least a 10 fold increased cytotoxicity relative to non-modified NK cells. In an embodiment, using a described leucine zipper approach provides an improved property of modified cells, relative to cells modified using a SpyTag/SpyCatcher approach. This approach is known in the art, for example, from Zakeri et al., Proc Natl Acad Sci USA. 2012 Mar. 20; 109(12): E690-E697, the disclosure of which is incorporated herein by reference. Thus, in embodiments, the described GE enzymes are not coupled to the described cells using an intact or split fibronectin-binding protein. In embodiments, the described GE enzymes are not coupled to a plasma membrane by an antibody.

The modified cells can be used in combination with any other cancer therapy, including but not necessarily limited to chemotherapeutic agents, therapeutic antibodies, including but not limited to checkpoint inhibitors, and adoptive immunotherapies.

### EXAMPLES

The following Examples are intended to illustrate but not limit the disclosure. Certain of the Examples involve a technique referred to herein as Ring-SAIM. In this regard, several imaging strategies have been developed to image the nanoscale dimensions and structural organization of the glycocalyx^{29,36-38}. One of these technologies, called Scanning Angle Interference Microscopy (SAIM), is a localization microscopy technique based on the principles of Fluorescent Interference Contrast Microscopy (FLIC) whereby standing waves of excitation light are used to axially localize fluorescently labelled structures of interest with nanoscale precision^{24,39-42}. To improve the precision of SAIM for glycocalyx materials research, the disclosure includes use of a pair of high-speed, galvanometer-controlled mirrors to generate a revolving circle, or “ring”, of excitation light at defined sample incidence angles, as described in Colville, et al., Scientific Reports 9, 1-13 (2019), the disclosure of which is incorporated herein by reference. The described Ring-SAIM approach improves sample illumination and, thus, the precision of measurement compared to standard SAIM implementations⁴⁰.

### Example 1

### Physical Properties of the Mucin Polymer Brush

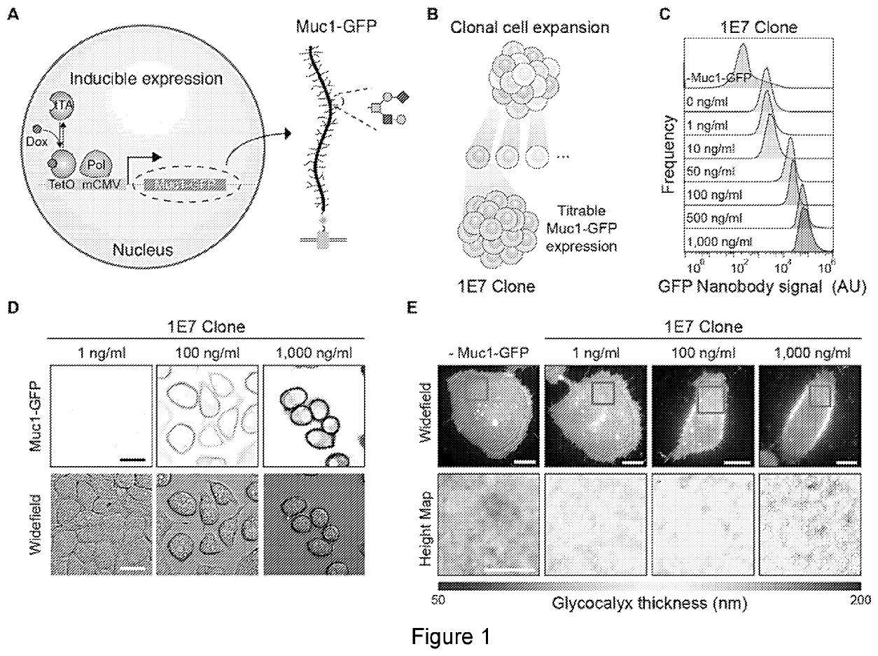

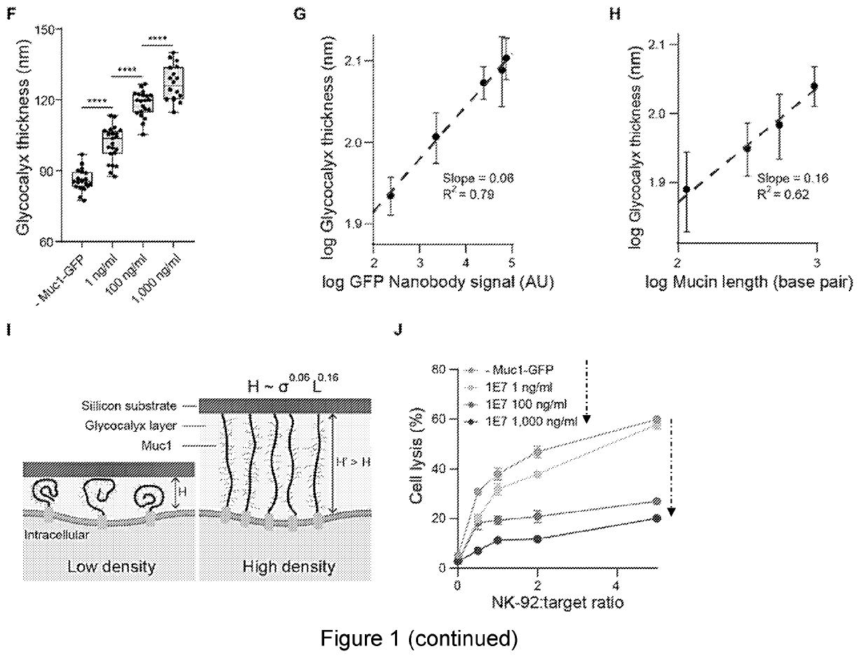

Cell-surface mucins are anchored to the plasma membrane on one end by their transmembrane domain, forming a brush-like structure⁴³⁻⁴⁵. Polymer chains are predicted to extend out at higher surface densities due to steric repulsions in a more crowded structure⁴⁶. In tumors and cell lines that overexpress cancer-associated mucins, high heterogeneity in the cell surface levels is typical⁴⁷⁻⁴⁸. To test how the expression level of mucin affects the material thickness of the glycocalyx, the disclosure provides a cellular model with highly titratable cell-surface levels of the cancer-associated mucin, Muc1. The disclosure provides an expression cassette for Muc1-GFP with 42 tandem repeats (TRs) under the control of a tetracycline-inducible promoter (FIG. 1A). We initially generated a polyclonal cell population through stable integration of the expression cassette in epithelial cells. In the polyclonal population, the ability to titrate mucin expression in a dose-dependent manner with doxycycline was limited by the large cell-to-cell variability in expression levels and the variability of the doxycycline response curves. To overcome this challenge, we clonally expanded the cell pool and identified multiple clones that exhibited accurate dose responses to doxycycline induction. One of these clonal lines, referred to here as 1E7, was selected as a representative model for subsequent experiments (FIG. 1B-1D).

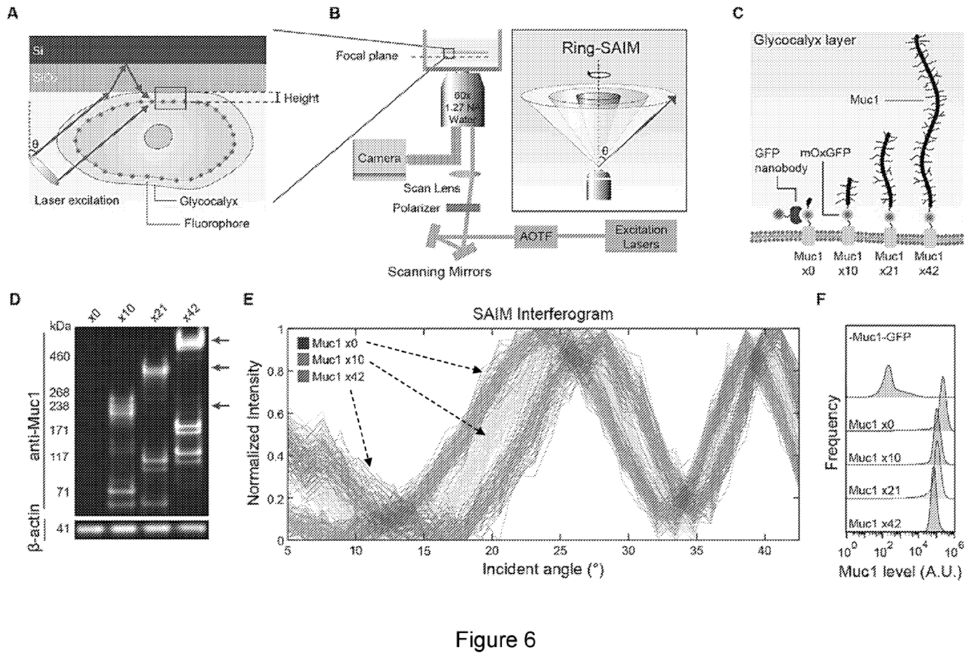

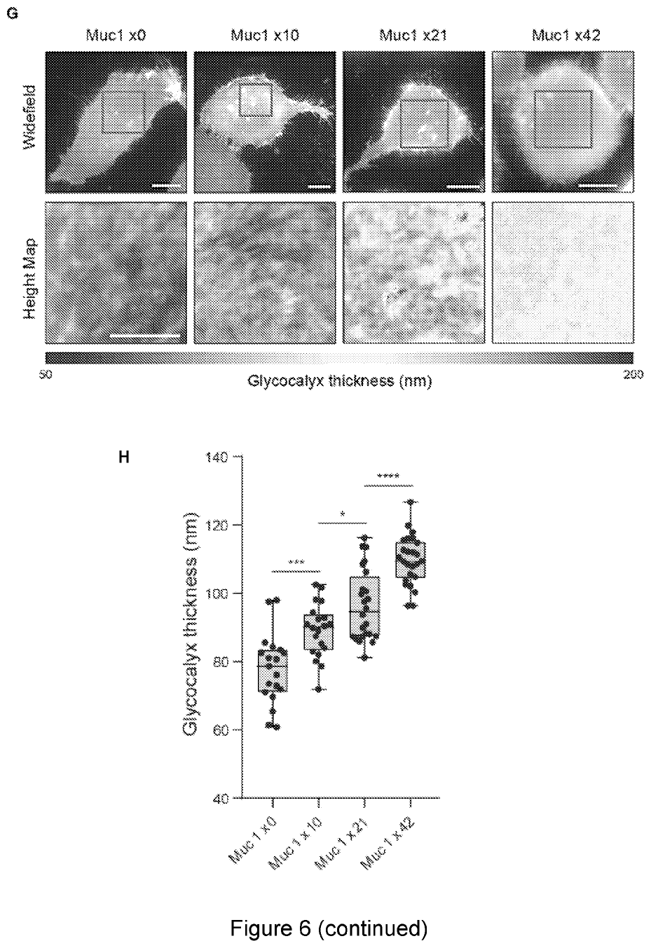

Noting that the height and compressibility of a polymer brush are known to scale with the polymer grafting density and polymer chain length^{49,50}, the disclosure provides a description of quantitative relationships that described the thickening of the glycocalyx by increased expression of cell-surface mucins. We first validated that the Ring-SAIM setup could detect nanometer-scale changes in the glycocalyx thickness of live cells using Muc1 constructs of varying size (FIG. 6). After confirming the ability of Ring-SAIM to detect changes in glycocalyx thickness of approximately 10-nm (FIG. 6H), we measured the thickness of the glycocalyx in 1E7 cells at varying doxycycline induction levels. We observed a progressive thickening of the glycocalyx with higher induction levels of Muc1 (FIG. 1E). The glycocalyx thickness ranged from 86.0±4.58 nm for cells without Muc1-GFP expression to 126.8±7.45 nm for cells at saturating levels of Muc1-GFP induction (FIG. 1F). The data revealed a clear power-law dependence of glycocalyx thickness on the Muc1-GFP surface density with a scaling exponent of 0.06 (FIG. 1G). To construct this relationship, we used the GFP nanobody signal as a proportionate quantity to the Muc1-GFP surface level. The glycocalyx thickness also had a clear power-law dependence on the length of the mucin with a scaling exponent of approximately 0.16 (FIG. 1H). Thus, the glycocalyx thickness weakly scaled with mucin surface density and more strongly scaled with mucin backbone length (FIG. 1I). These relationships provide a framework for understanding how the heterogeneity in mucin expression level and variation in the number of TRs could influence the thickness and barrier properties of the glycocalyx (FIG. 1I).

### Example 2

### The Mucin Brush Resists NK-Cell Attack

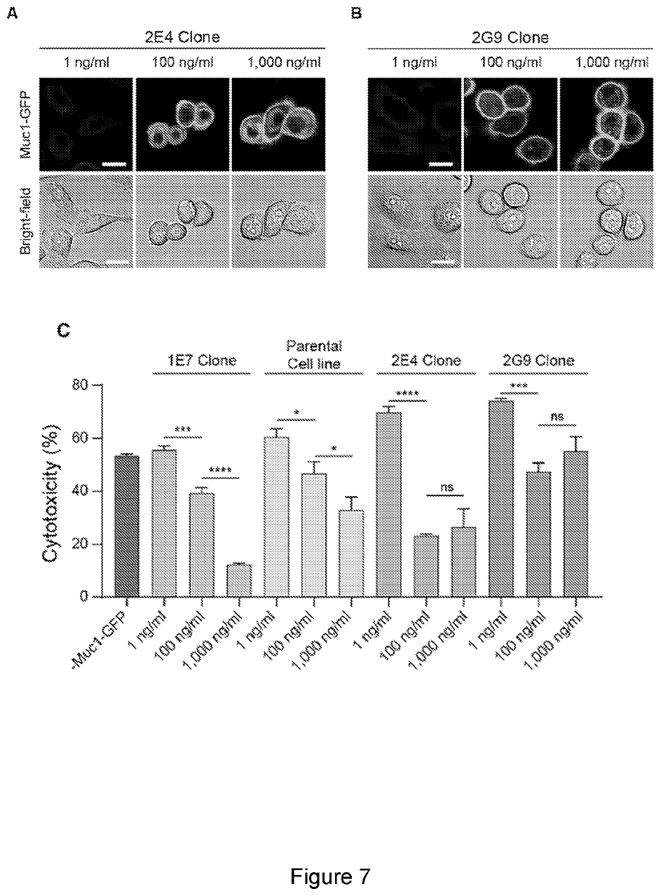

The Ring-SAIM measurements in the 1E7 clone indicated how mucin chain length and density would resist close cellular contact with a surface (FIGS. 1G and 1H). We tested whether a mucin brush could similarly resist effector cell engagement with a target cell, a process that requires close, receptor-mediated interactions between cells. We considered immune cell attack by NK-92, a well-characterized NK cell line that has demonstrated promising anticancer activities in human clinical trials^{51,52}. NK-92 cytotoxicity against target cells was reduced by Muc1-GFP expression in a dose-dependent manner (FIG. 1J). NK-92 cells do not express appreciable levels of inhibitory Siglec-7 and Siglec-9, the two primary receptors which have been implicated in NK suppression by Muc1 O-glycans^{7,16,53}, raising the possibility that the protection by mucins was mediated by a physical mechanism. To confirm that the results were not a clonal artifact limited to the 1E7 cell line, we repeated the NK-92 cytotoxicity assay against two additional Muc1-GFP expressing clones, as well as the parental polyclonal cell line from which the 1E7 clone was derived. We observed a similar trend of enhanced resistance to NK-cell attack with increasing Muc1-GFP expression in the parental line and all clones tested (FIG. 7).

### Example 3

### Regulation of Glycocalyx Material Thickness by Mucin-Type O-Glycosylation and Multivalent Galectins

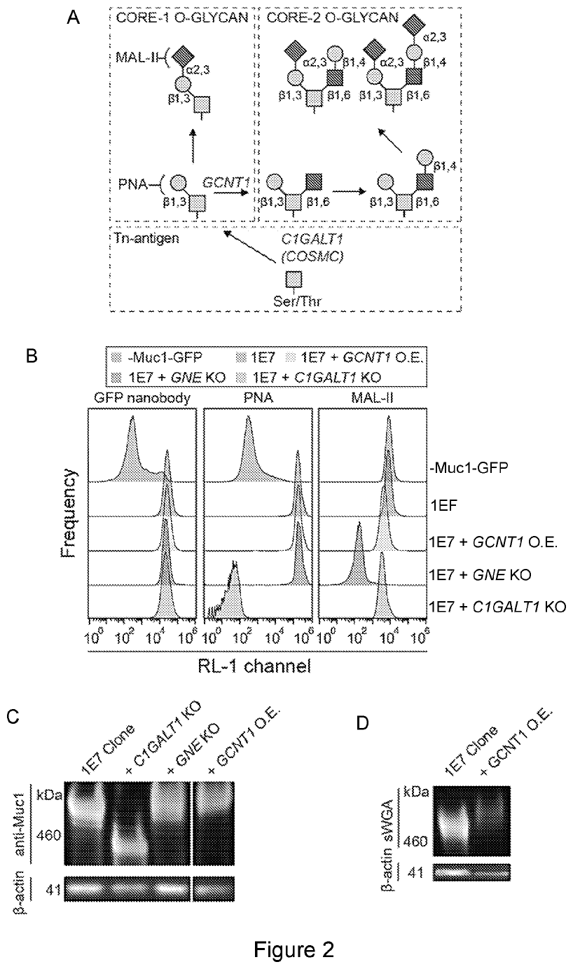

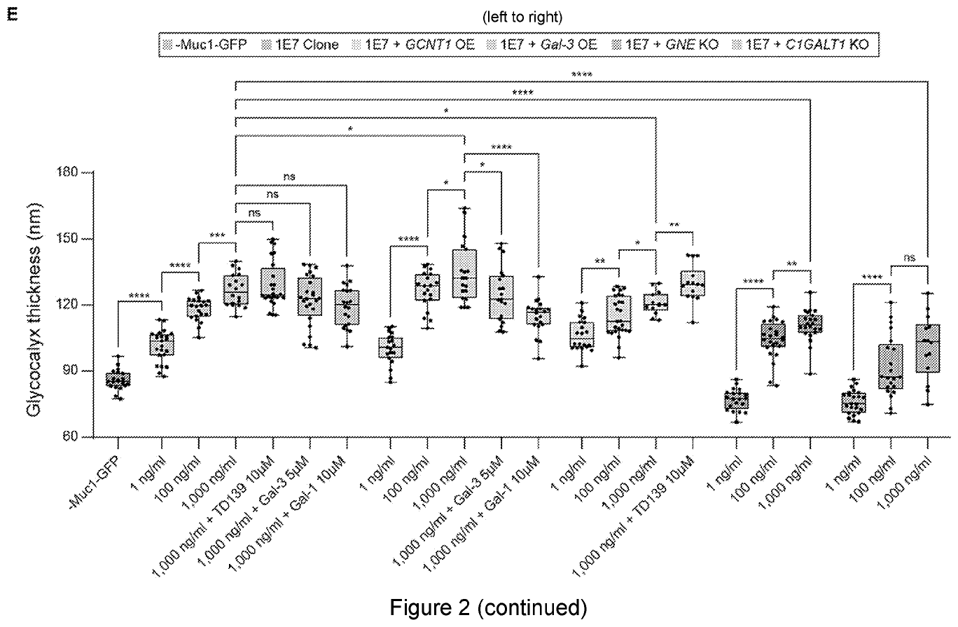

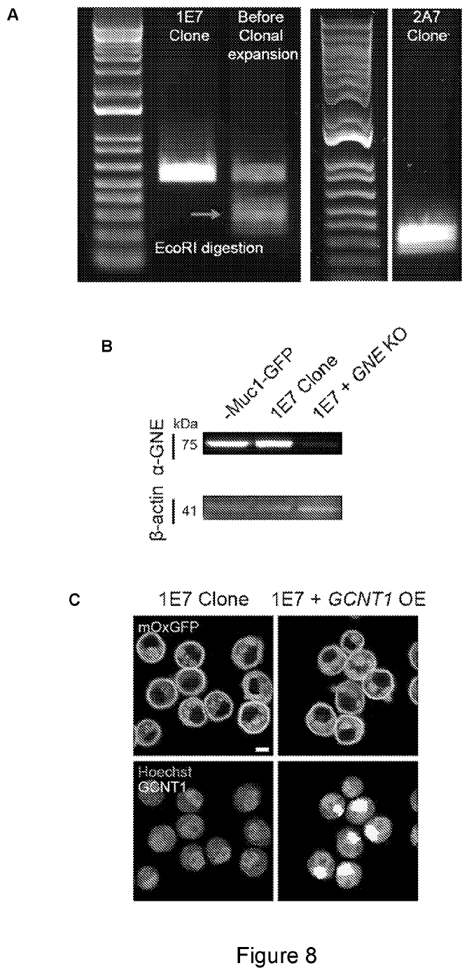

This example provides a description of how specific molecular attributes of mucins contribute to the ensemble materials properties of the glycocalyx and, thus, protect against immune cell attack. For bottlebrush copolymers with densely grafted side chains, such as mucins, the size and charge of the side chains can strongly influence the polymer rigidity and persistence length^{54,55}. Thus, we investigated how O-glycan truncation, elongation, and sialylation each contribute to the material thickness of the glycocalyx in 1E7 cells. 0-glycans were truncated in 1E7 cells through CRISPR/Cas9 mediated knockout (KO) of the C1GALT1 gene, which encodes the Core 1 synthase that governs the extension of α-GalNAc (FIG. 2A). Sialylation was ablated by CRISPR/Cas9 KO of the GNE gene, which encodes the glucosamine (UDP-N-acetyl)-2-epimerase/N-acetylmannosamine kinase, an essential enzyme in CMP-sialic acid biosynthesis. The 1E7 C1GALT1 and GNE KO cells were each clonally expanded to isolate homozygous KO lines (FIGS. 8A and 8B). Glucosaminyl (N-Acetyl) Transferase 1 (GCNT1) was overexpressed to increase O-glycan extension through Core-II elongation (FIGS. 2A and 8C). Analysis by flow cytometry showed similar cell-surface levels of Muc1-GFP across the glycoengineered cell line panel, indicating that manipulation of glycosylation did not alter Muc1-GFP surface expression (FIG. 2B). Disruption of Core-I O-glycan extension or sialylation in the KO lines was confirmed by flow cytometry using appropriate lectin probes (FIG. 2B). Since there are no direct immunochemical or lectin probes for Core-II O-glycans, we instead confirmed increased elongation of O-glycans in GCNT1 overexpressing cells by SDS-PAGE (FIGS. 2C and 2D). The apparent molecular weight of Muc1-GFP observed by SDS-PAGE was substantially lower in C1GALT1 KO cells and appreciably higher in GCNT1 overexpressing cells compared to Muc1-GFP in wild-type cells, consistent with the presence of shorter and longer glycan side chains, respectively (FIGS. 2C and 2D).

Utilizing Ring-SAIM, we measured the material thickness of the glycocalyx across the glycoengineered cell panel. At each Muc1-GFP induction level, the glycocalyx was significantly thinner in C1GALT1 and GNE KO cells compared to wild-type 1E7 cells (FIG. 2E). At 1,000 ng/mL doxycycline induction, KO of C1GALT1 had the largest effect on the glycocalyx, reducing its material thickness by 25.1 nm compared to wild-type 1E7 cells. KO of GNE reduced the average glycocalyx thickness by 16 nm, whereas GCNT1 overexpression increased the average glycocalyx thickness by 7.6 nm at the same induction level (FIG. 2E). Notably, even with the truncation of O-glycans or ablation of sialylation, the thickness of the glycocalyx increased with increasing surface density (FIG. 2E). Thus, even minimally glycosylated mucin polymers could resist close cell-to-surface contact when expressed at sufficiently high levels.

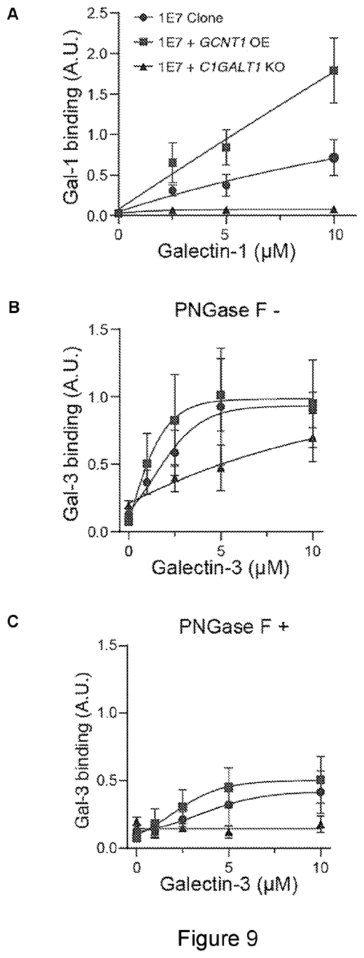

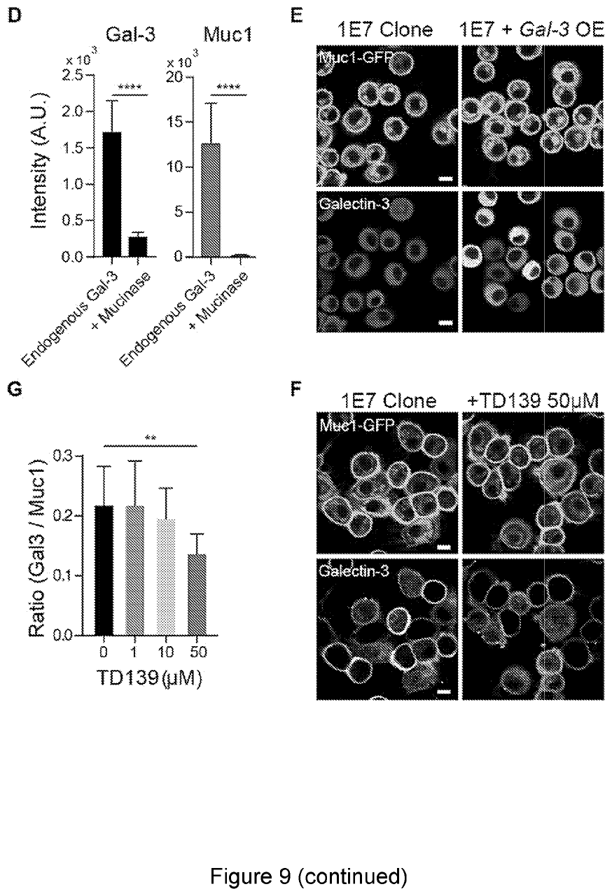

Multivalent galectins have been proposed to crosslink and increase the barrier properties of cell-surface mucins⁵⁶. The disclosure provides data demonstrating specific interactions of galectin-1 and -3 with Muc1-GFP on 1E7 cells (FIG. 9). Recombinant galectin-1 showed a preference for Core-II O-glycans, whereas recombinant galectin-3 bound specifically to Core-I O-glycan structures, as well as N-glycans (FIG. 9). Treatment of wild-type 1E7 cells with recombinant galectin-3, recombinant galectin-1, or 10 μm of the small-molecule galectin inhibitor TD139 (K_{d}=0.036 μM for galectin-3; K_{d}=2.2 μM for galectin-1) did not significantly change the material thickness of the glycocalyx at 1,000 ng/mL doxycycline induction (FIG. 2E). Likewise, the effect of galectin-3 overexpression on the glycocalyx thickness in 1E7 cells was either small or not statistically significant depending on the Muc1-GFP expression level (FIG. 2E). In 1E7 cells overexpressing GCNT1, treatment with recombinant galectin-3 slightly decreased the glycocalyx thickness by 9.5 nm compared to untreated cells (p=0.0308; FIG. 2E). Galectin-1 treatment had a more pronounced effect, reducing the glycocalyx thickness by 19.4 nm compared to untreated GCNT1 overexpressing cells (p=0.000003; FIG. 2E).

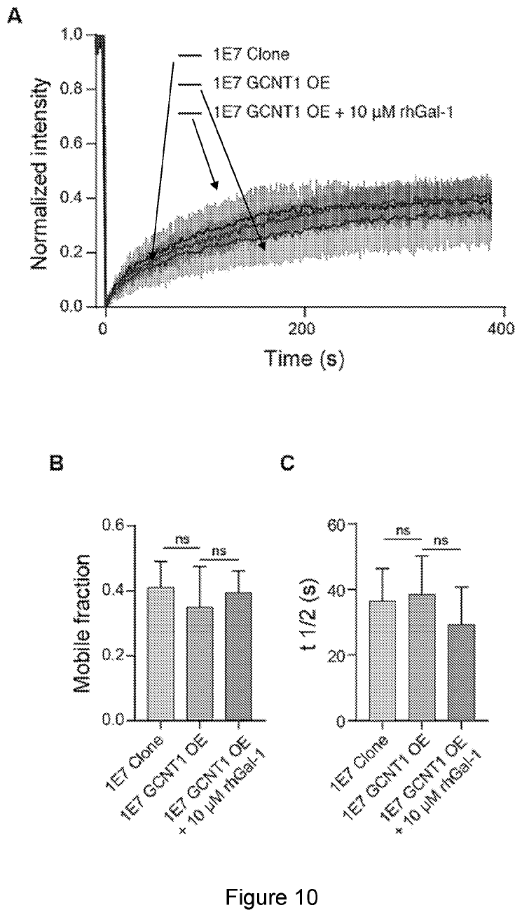

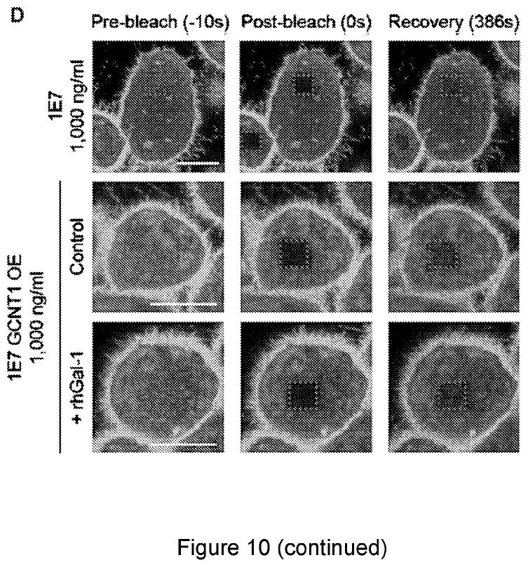

These observations were consistent with galectins restricting the free chain extension of mucins, presumably through either intermolecular or intramolecular crosslinking. Fluorescence recovery after photobleaching (FRAP) showed that a large percentage of mucins were mobile in the cell-surface brush, indicating that intermolecular crosslinking by galectin was modest (FIG. 10). Overall, the relatively small effects of the galectin manipulations compared to the larger effects observed with varying mucin surface density, length, and glycosylation pointed to chain entropy, and not crosslinking, as the major determinants of the glycocalyx material thickness at the substrate interface.

### Example 4

### The Material Thickness of the Glycocalyx Accounts for the Resistance to NK Cell Attack

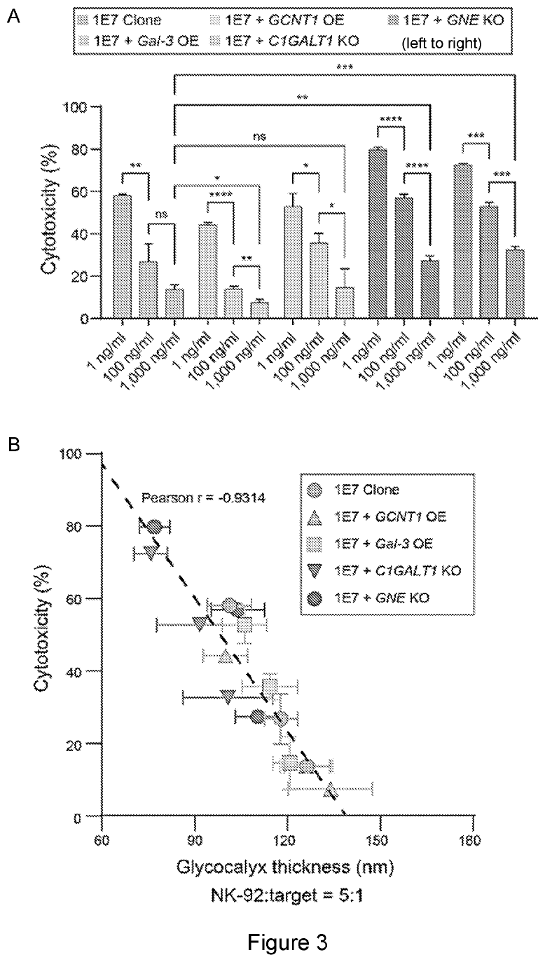

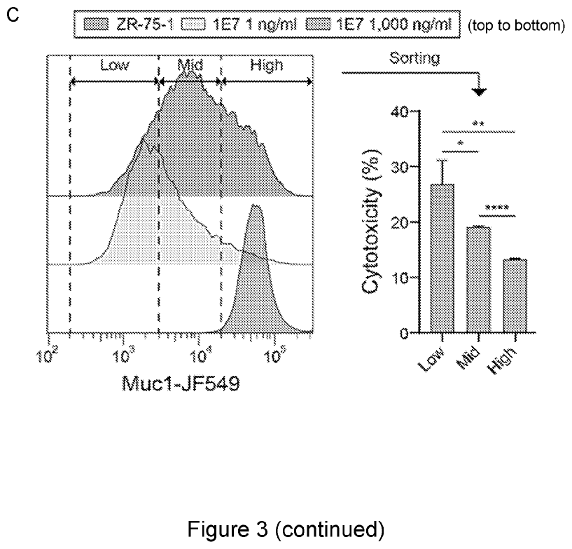

The described Ring-SAIM measurements and biochemical analyses provided guidance as to how the material and biochemical state of the glycocalyx could be manipulated by tuning mucin density and glycosylation. With this information, the disclosure provides an analysis of how specific glycocalyx states protect against immune cell attack. 1E7 cells at varying Muc1-GFP induction levels and their glycoengineered progenies were co-incubated with NK-92 to assess cytotoxicity. Across the glycoengineered panel, NK-92 mediated cytotoxicity against the mucin-expressing target cells was progressively reduced at higher doxycycline induction levels (FIG. 3A). Mucins with truncated and less sialylated O-glycans provided less protection against NK-92 attack (FIG. 3A). Thus, and without intending to be bound by any particular theory, resistance to NK cell attack appeared to correlate with conditions that favored extension of a thicker glycocalyx.

To assess the relationship between resistance to immune cell attack and glycocalyx material thickness, the NK-mediated cytotoxicity reported in FIG. 3A was plotted against the measured glycocalyx thickness reported in FIG. 2E for each of the glycoengineered cell type and Muc1-GFP induction level. A strong and nearly perfect inverse correlation between cytotoxicity and glycocalyx material thickness was observed across the combined dataset representing different mucin expression levels and glycosylation patterns (Pearson coefficient=−0.94; FIG. 3B). The cytotoxicity data were fit to a linear model dependent on a single variable: the glycocalyx thickness (FIG. 3B). Surprisingly, we found that the glycocalyx thickness explained 86% of the variation in NK mediated cytotoxicity against the full glycoengineered cell panel, which presents glycocalyces with widely varying glycosylation patterns and biochemical attributes.

We next tested the ability of NK cells to attack cancer cell lines that endogenously express cell-surface mucins. We benchmarked the Muc1 surface levels of a luminal A breast cancer cell line, ZR-75-1, against the 1E7 cell line. Non-permeabilized cells were probed with conventional Muc monoclonal antibodies and analyzed by flow cytometry. We constructed distributions for Muc surface levels in 1E7 cells at minimal and saturating Muc1-GFP induction (1 ng/mL and 1,000 ng/mL doxycycline) to define standardized gates for low, moderate, and high Muc expression that could be applied to categorize other cell lines. The Muc1 surface levels were highly heterogeneous in the ZR-75-1 cell line and fully overlapped with the 1E7 low, medium, and high expression benchmarks (FIG. 3C). Notably, these results confirmed that the Muc1-GFP levels investigated in the model 1E7 cell system were within the bounds of naturally varying Muc1 levels in tumor cells. Similar to results with 1E7 cells, NK cell cytotoxicity against the ZR-75-1 cells decreased in a graded fashion with increased Muc surface levels (FIG. 3C). Together, these results strongly implicate the glycocalyx material structure as an important determinant of cellular resistance to immune cell attack.

### Example 5

Tethering Mucinase to NK Cells can Dramatically Increase their Cytotoxicity Against Mucin-Expressing Target Cells.

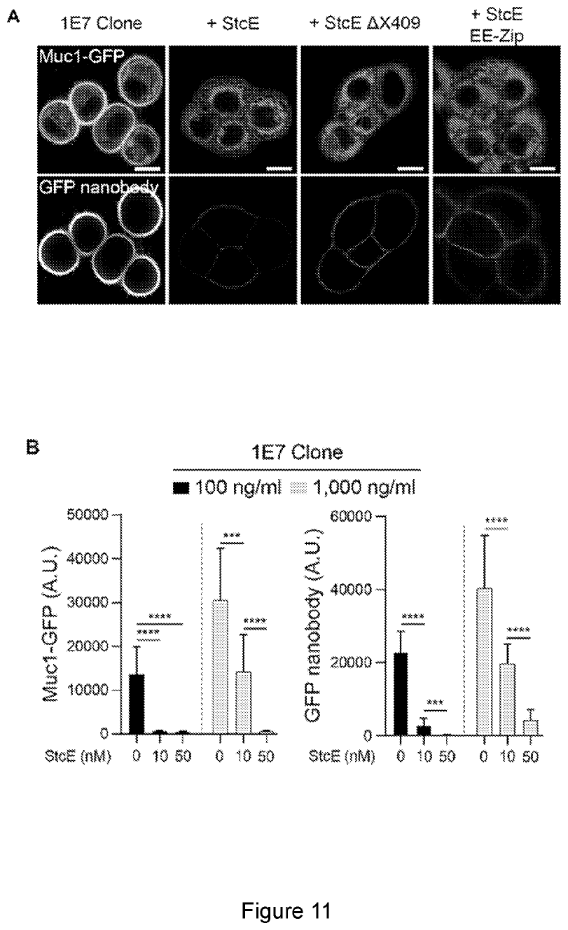

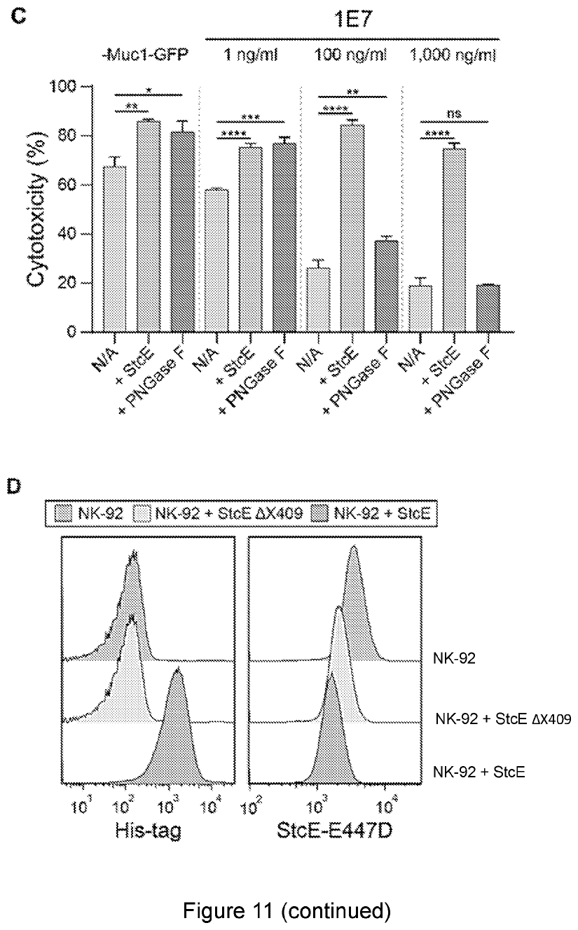

Enterohemorrhagic Escherichia coli (EHEC) utilize the mucin-specific metalloprotease, StcE, to breach the gut mucosal barrier and adhere to intestinal cells⁶⁰. The disclosure includes effector immune equipped with StcE mucinase, which is extendable to other mucinases as further described herein, to breach the mucin barrier on target cells⁶¹. The StcE mucinase was highly effective at removing the cell-surface mucin brush from 1E7 cells (FIGS. 11A and 11B), rendering the cells more susceptible to NK-92 mediated attack (FIG. 11C). The disclosure includes various approaches for tethering the StcE mucinase could to the NK-92 cell surface. The same approach accordingly applies to tethering other glycocalyx modifying enzymes to NK cells and other immune cells as described herein.

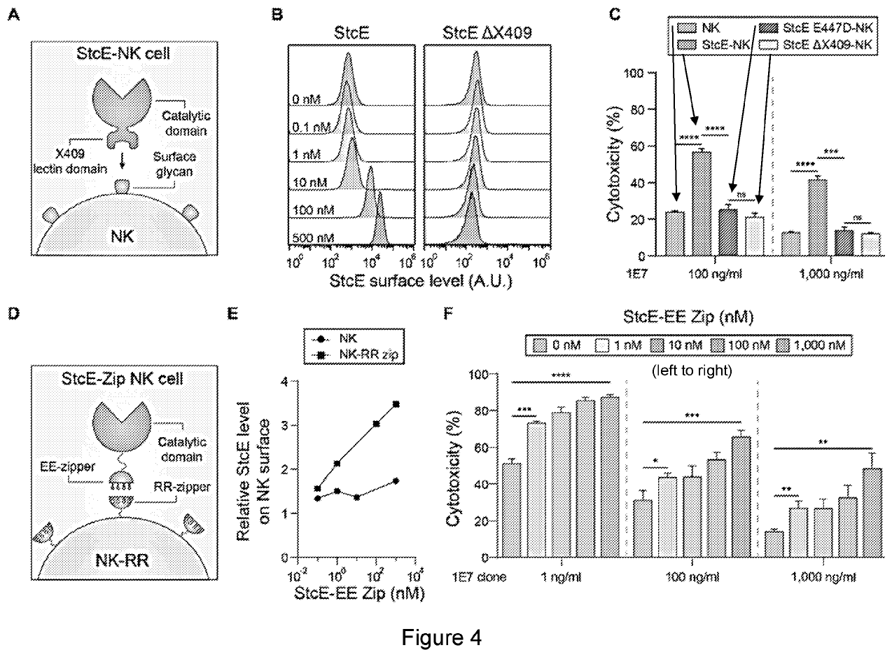

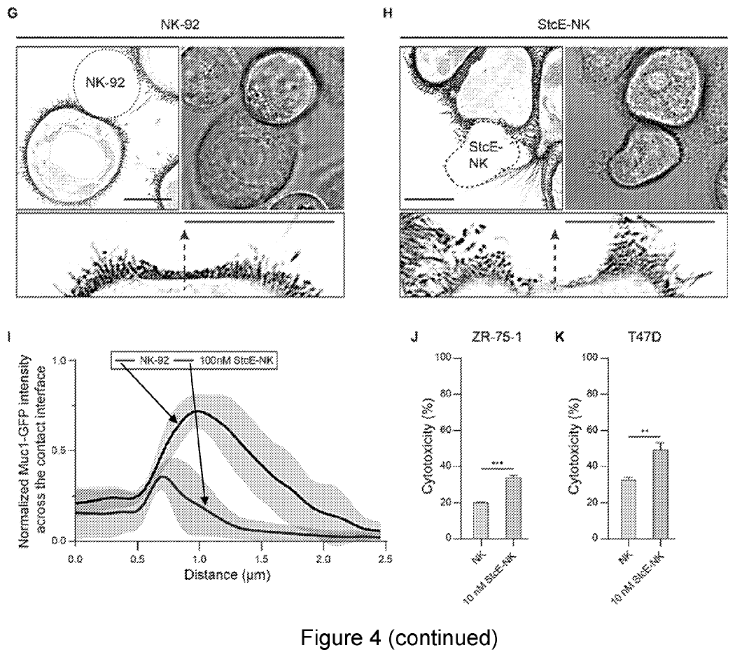

The StcE mucinase possesses a C-terminal lectin domain, referred to as X409, in addition to its catalytic domain¹². We tested whether the StcE mucinase would specifically bind to the NK-92 cell surface and remain bound without an additional molecular tether. The recombinant StcE bound stably to the NK-92 cell surface and was retained for prolonged periods even after stringent washing with buffer (FIGS. 4A and 4B). StcE-ΔX409 with a deleted C-terminal lectin domain was not retained on the NK-92 surface, indicating that the X409 domain was the primary binding module to the NK-92 surface (FIG. 4B).

The StcE-equipped NK-92 (StcE-NK) cells were considerably more effective at killing mucin-expressing target cells than the unmodified NK-92 cells (FIG. 4C). Control experiments with the catalytically dead StcE-E447D mucinase confirmed that the enzymatic activity of StcE was required for the enhancement of cytotoxicity (FIG. 4C). Additional controls indicated that the surface tethering of StcE to the NK surface was required for the enhanced cytotoxicity against mucin-expressing targets. Indeed, the cytolytic activity of NK cells was not significantly enhanced by treatment with StcE-ΔX409, which we confirmed could actively digest Muc but did not remain tethered to the NK surface (FIGS. 4C and 11A). The NK-92 cells possessed a small number of mucin-domain containing proteins that were trimmed similarly by wild-type StcE and StcE-ΔX409 (FIG. 11D). The inability of StcE-ΔX409 treatment to enhance the cytolytic activity of NK cells indicated that the StcE mucinase treatment likely did not directly enhance the latent activity of NK cells through the trimming of mucin domain proteins from the NK cells surface (FIG. 4C).

The disclosure also includes a modular strategy whereby StcE or other glycocalyx-editing (GE) enzymes can be displayed on the NK cell surface, which is considered to be extendable to other types of immune cells as described herein. The X409 lectin domain of StcE was swapped for the EE leucine zipper and the new fusion protein, StcE-EE Zip, was produced recombinantly (FIG. 4D). We confirmed that StcE-EE Zip could cleave Muc from the cell surface similarly to wild-type StcE (FIG. 11A). StcE-EE Zip was subsequently coupled to the NK surface through the cognate RR leucine zipper, which was stably expressed as a fusion with a transmembrane anchor in the NK cells (FIGS. 4D and 4E). The StcE-Zip NK cells displayed significantly enhanced cytotoxicity against mucin-bearing target cells (FIG. 4F). Cytotoxicity against the 1E7 cells was qualitatively similar for StcE-Zip NK cell and StcE-NK cells in which StcE was coupled to the NK surface through the native X409 lectin domain, indicating that the performance enhancement was agnostic to the precise StcE coupling strategy (FIGS. 4C and 4F).

The disclosure includes an analysis of the contact zone between the NK cells and their targets. We observed a localized disruption of the mucin barrier at the interface between StcE-NK cells and target cells. Visible deflection of microvilli on 1E7 cells in the contact zone with both unmodified NK-92 and StcE-NK indicated that both the wild-type and engineered cells could form viable mechanical connections with the target cells (FIGS. 4G and 4H). However, a high density of Muc1-GFP and microvilli persisted in the contact zone with unmodified NK-92 cells (FIG. 4G). Significantly lower Muc1-GFP levels and fewer microvilli were observed in the contact zone with StcE-NK cells (FIGS. 4H and 4I). These results were consistent with the surface-tethered StcE locally cleaving mucin or otherwise aiding in the segregation of cell-surface mucin from the contact interface, as well as disrupting microvilli (FIG. 4I).

Surface display of StcE also improved NK attack against cancer cell lines expressing endogenous Muc1. StcE-NK cells attacked the ZR-75-1 cells more aggressively compared to unmodified NK-92 cells (FIG. 4J). Similar results were observed in T47D cells, another luminal A breast cancer line that expresses moderate to high levels of endogenous Muc (FIG. 4K). Taken together, our results instead suggested that StcE display likely improved the efficacy of NK cell attack by overcoming the mucin physical barrier.

### Example 6

### CAR-NK and StcE-NK can Overcome the Mucin Barrier

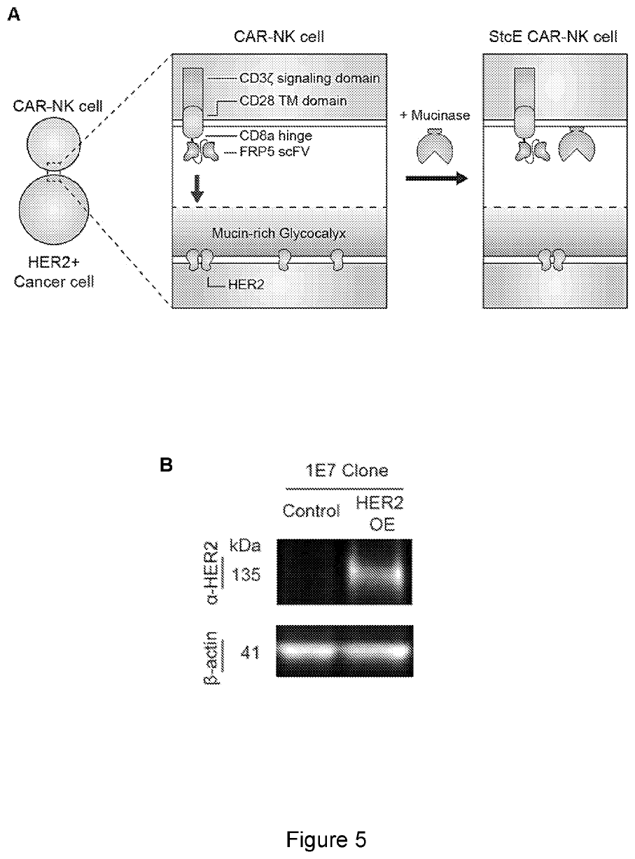

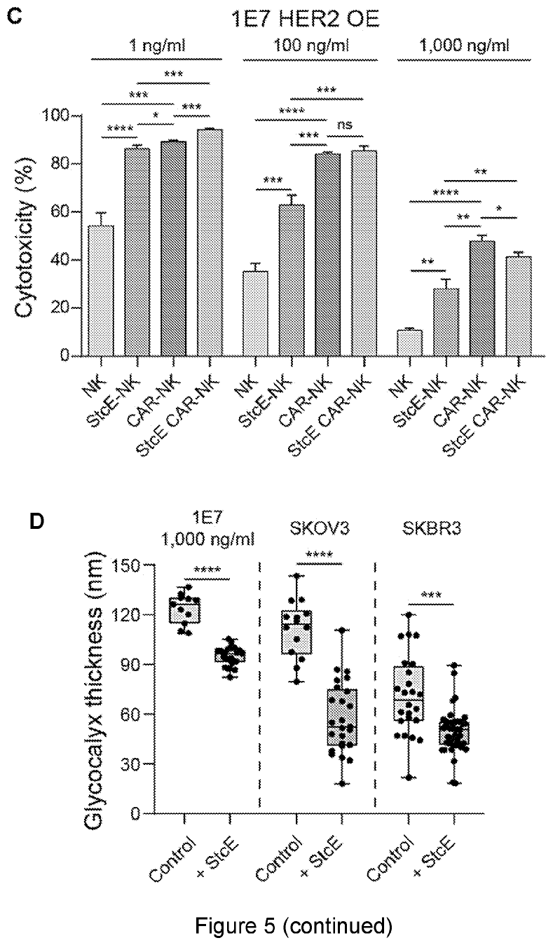

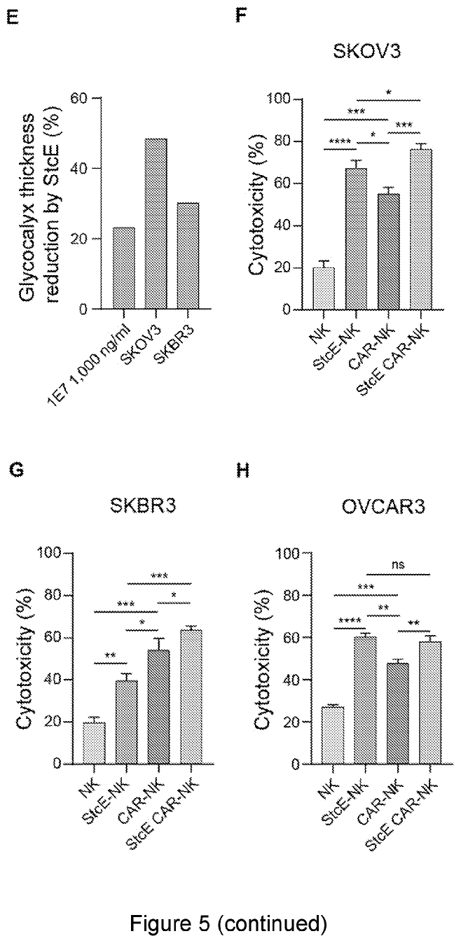

Chimeric antigen receptors (CAR) engagement in CAR-NK cells can override inhibitory signals deployed by tumor cells and directly trigger the effector cells' intrinsic cytolytic effector functions, as well as the release of pro-inflammatory cytokines⁶³. We next tested whether a mucin barrier would also protect against CAR-NK attack. We engineered the NK-92 cell line to express a humanized anti-HER2 CAR with CD28 and CD3ζ signaling domains (FIG. 5A). This CAR was previously validated in NK-92 cells, which are currently under investigation in human clinical trials for glioblastoma^{64,65}.

To test the ability of the glycocalyx to physically resist CAR-NK cell attack, HER2 was overexpressed in the 1E7 cell line (FIG. 5B). CAR-NK cells were more effective at killing HER2 1E7 cells than control NK-92 cells, although the mucin barrier still provided resistance against CAR-NK attack (FIG. 5C). Cytotoxicity mediated by CAR-NK cells against HER2 1E7 cells dropped from 89.40% at 1 ng/mL doxycycline induction of Muc1-GFP to 47.92% at 1,000 ng/mL induction (FIG. 5C). CAR-NK cells killed the HER2 1E7 targets with moderately higher efficiency compared to StcE-NK cells (FIG. 5C). StcE CAR-NK cells, which were equipped with mucinase and the engineered immune receptor, killed the 1E7 targets with similar efficiency as CAR-NK cells (FIGS. 5A and 5C).

We evaluated the killing efficiency of StcE-NK, CAR-NK, and StcE CAR-NK cells against cancer cells lines that endogenously express HER2 and cell-surface mucin. We first tested the ovarian cancer cell line, SKOV3, which expresses Muc1 and another large mucin called Podocalyxin^{66,67}. SKOV3 cells had a thick glycocalyx of approximately 120 nm that was reduced to approximately 60 nm following mucinase treatment, confirming that cell-surface mucins were predominant structural elements of the glycocalyx of these cells (FIG. 5D). StcE-NK cells had more than three-fold higher cytotoxicity against SKOV3 targets than unmodified NK-92 cells, killing the targets with an even greater efficiency than CAR-NK cells (FIG. 5F). We also tested SKBR3 breast cancer cells, which express some Muc1, but overall had a thinner glycocalyx than 1E7 and SKOV3⁶⁸. Against these targets, CAR-NK cells were more effective at killing than StcE-NK, although StcE-NK did kill a significantly higher percentage of SKBR3 cells compared to unmodified NK-92. Equipping the NK cells with both StcE and the CAR resulted in a modest enhancement of cytotoxicity compared to the StcE-NK and CAR-NK cells (FIG. 5G). We also evaluated ovarian cancer OVCAR3 cells, which express Muc and the very large cell-surface mucin, Muc16^{69,70} (FIG. 5H). We could not accurately measure the glycocalyx thickness of OVCAR3 cells due to the poor adhesion of these cells to the SAIM substrate, which was possibly due to a thick mucin barrier on these cells. Mirroring the results for SKOV3, StcE-NK cells were more effective at killing these targets than CAR-NK. Collectively, these results demonstrate that immune cell engineering can help overcome the resistance to cellular attack posed by the mucin barrier.

### Example 7

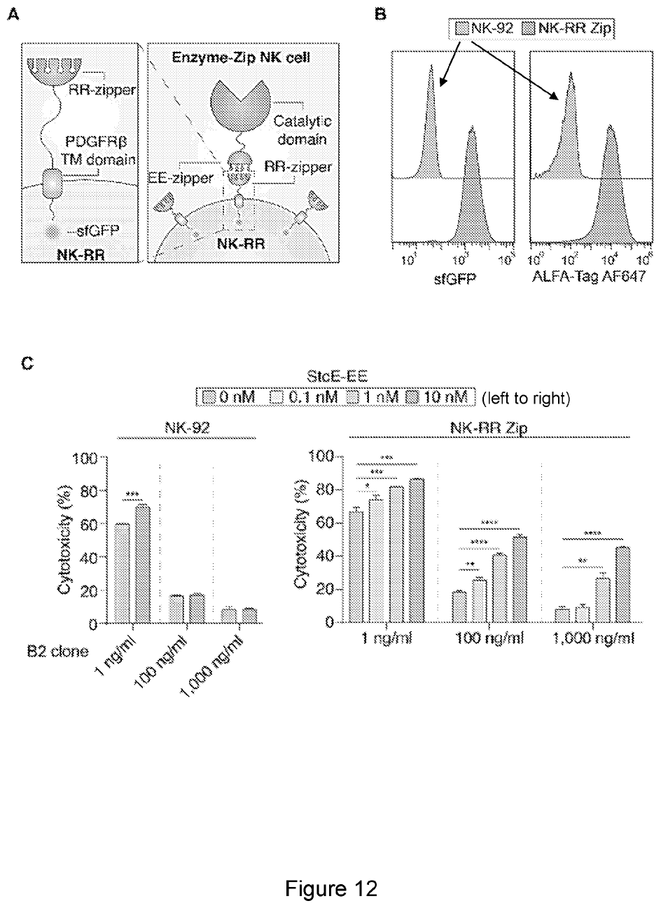

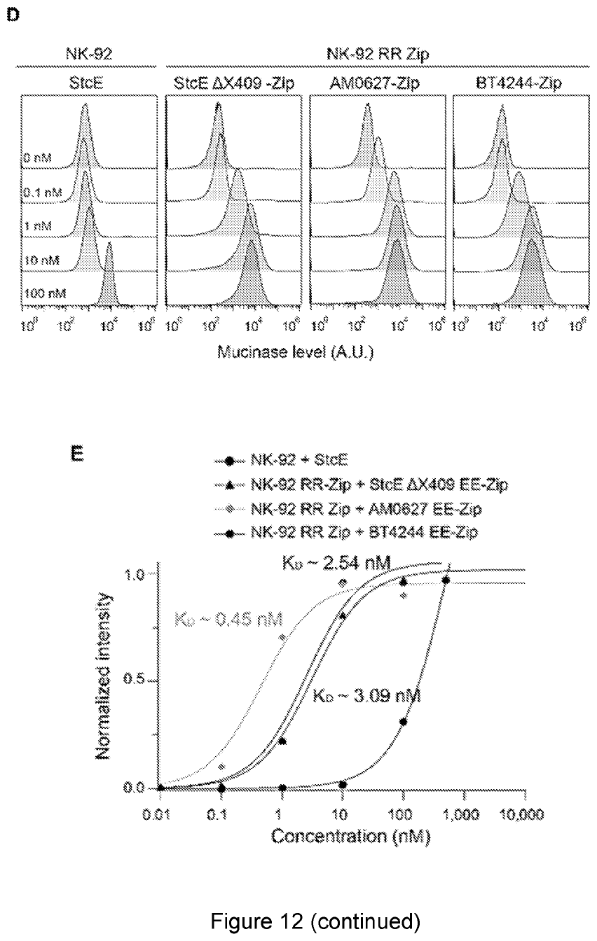

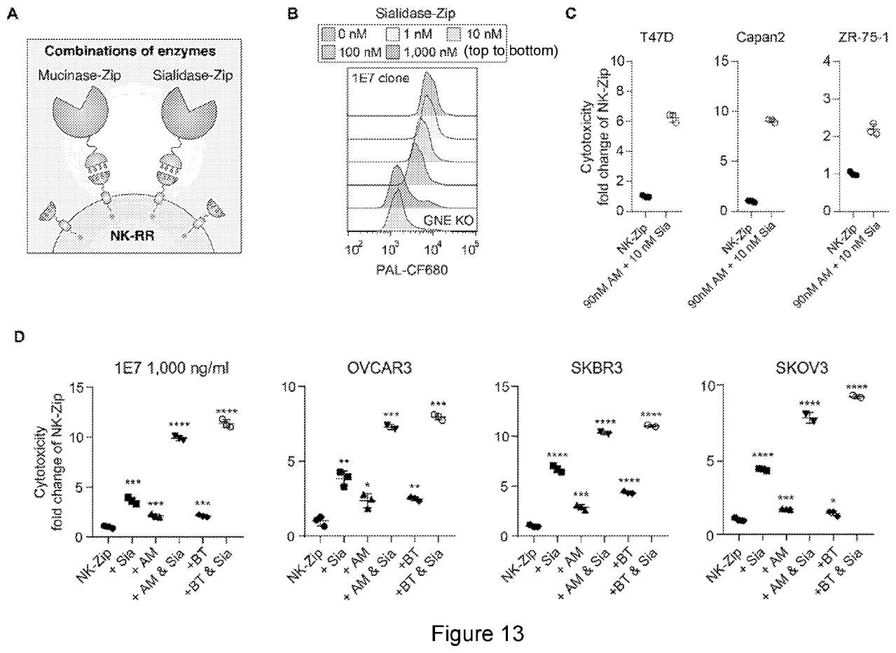

The disclosure includes additional examples showing the modularity of the leucine-zipper based coupling strategy for display of mucinases and glycosidases, individually or in combination, on the immune cell surface. In an embodiment, a fusion protein comprised of the RR leucine zipper, an extracellular ALFA-Tag, a type I transmembrane domain (from PDGFR), and an intracellular superfolder GFP (sfGFP) was genetically encoded and stably expressed in NK-92 cells (FIGS. 12A and 12B). The recombinant StcE-EE Zip described in Example 5 was subsequently coupled to the NK surface through noncovalent binding of the EE and RR leucine zippers. The StcE couple NK cells had improved cytotoxicity against epithelial cells expressing Muc or Muc1-GFP (FIG. 12C). We next demonstrated that other mucinases could similarly be coupled to the NK surface using leucine zippers. The mucinases BT4244 from Bacteroides thetaiotaomicron and M60-like protease AM0627 from Akkermansia muciniphila, which preferentially cleaves mucins with truncated, cancer-associated O-glycans⁷⁶, were recombinantly generated as fusions with the EE leucine zipper. The BT4244 and AM0627 proteins coupled to the surface of NK-92 cells expressing the membrane-anchored RR leucine zipper with nanomolar affinity (FIGS. 12D and 12E). The NK-92 cells coupled with BT4244 or AM0627 had enhanced cytotoxicity against 1E7 cells and OVCAR3, SKBR3, and SKOV3 cancer cells compared to NK-92 cells without the enzymes (FIG. 13D).

We next demonstrated that the cytotoxicity of NK cells could be enhanced through coupling of more than one type of enzyme on the immune cell surface. We generated recombinant V. cholerae sialidase fused to the EE leucine zipper. The recombinant protein was anchored to the surface of NK-92 cells expressing the membrane-anchored RR leucine (FIG. 13B). Engineering of NK cells with the sialidase improved their cytotoxicity against 1E7 cells and OVCAR3, SKBR3, and SKOV3 cancer cells (FIG. 13D). We next coupled the EE leucine zipper sialidase to the NK cells simultaneously with either the EE leucine zipper BT4244 or AM0627, such that both sialidase and BT4244 or sialidase and AM0627 would be displayed on the NK surface. We demonstrated that engineering the NK surface to display both a mucinase and sialidase improved cytotoxicity against OVCAR3, SKBR3, and SKOV3 cancer cells compared to NK cells displaying only the mucinase or only the sialidase individually (FIG. 13D). The enhanced cytotoxicity of the mucinase plus sialidase equipped cells was additionally validated on T47D, Capan2, and ZR-75-1 cancer cells (FIG. 13C).

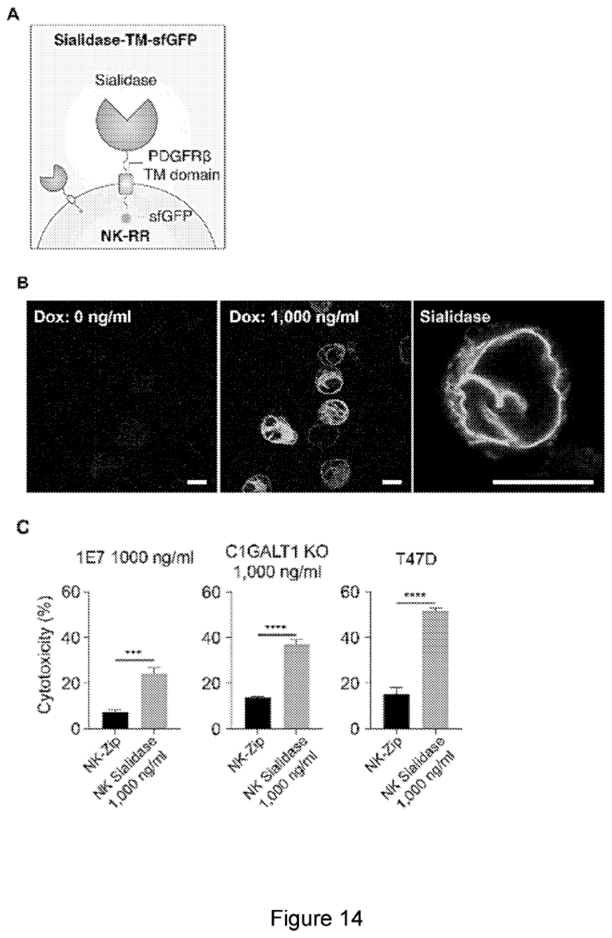

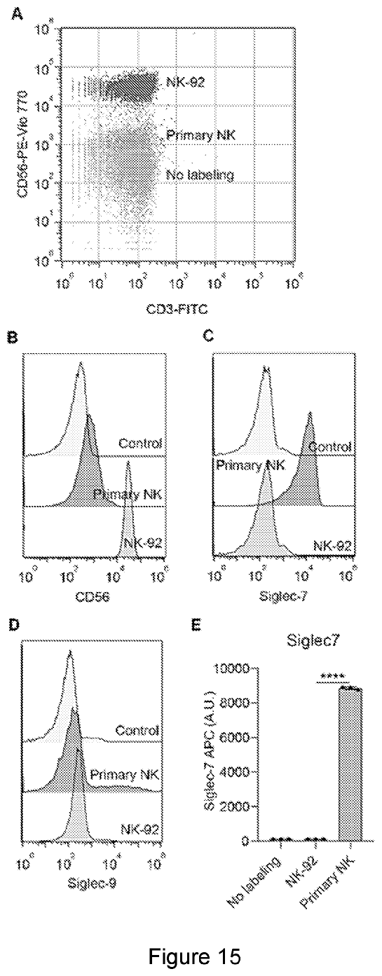

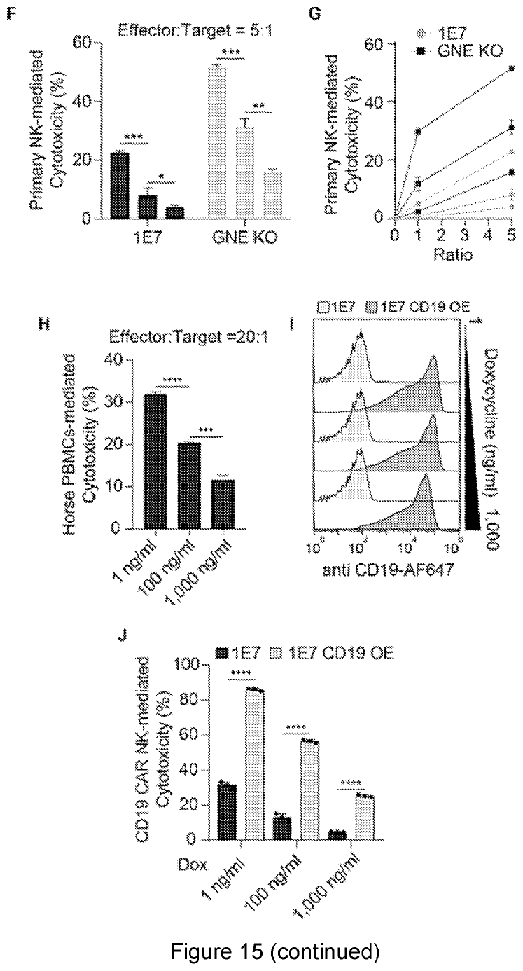

As an alternative strategy to leucine-zipper based coupling, we also demonstrate that V. cholerae sialidase could be genetically expressed on the immune cells surface. The sialidase was genetically encoded as a fusion with a eukaryotic signal peptide, the PDGFR transmembrane domain, and an intracellular sfGFP tag (FIG. 14A). The resulting cDNA was stably expressed in NK-92 cells. Confocal microscopy of the sfGFP tag confirmed that the sialidase was correctly localized on the NK-92 plasma membrane (FIG. 14B). We confirmed that the sialidase equipped NK cells showed increased cytotoxicity against 1E7 cells and T47D breast cancer cells (FIG. 14C). Thus, glycosidases could be functionally displayed on the immune cell surface through fusion with an appropriate transmembrane anchor.

### Summary of Examples 1-7.

It will be recognized from the foregoing Examples that the present disclosure demonstrates, among other aspects, that NK cell-mediated cytotoxicity shows a strong inverse relationship with the material thickness of the glycocalyx across widely varying states of glycosylation (FIG. 3B). By combining gene editing techniques and the Ring-SAIM approach for nanoscale optical characterization of the glycocalyx, the disclosure demonstrates how specific molecular attributes of mucins, including their glycosylation and backbone length, contribute to the expansion and material thickness of the glycocalyx and, thus, the barrier to immune cell attack. The disclosure demonstrates that equipping NK cells with a CAR or surface displayed StcE improved cytolytic activity against target cells.

In malignant conditions, differential glycosylation frequently results in the generation of truncated O-glycan structures, such as the Tn and sialyl-Tn antigens⁹. The described results reveal that mucins with truncated O-glycans can still resist close contact with an apposed surface when expressed at high levels. In general, increased mucin surface density can compensate for O-glycan truncation or loss of sialylation to maintain the structural integrity and thickness of the glycocalyx. Thus, the mucin barrier that is characterized in this disclosure may be broadly relevant to cancer types that overexpress mucins, even across the highly heterogeneous patterns of glycosylation that are observed among specific tumor types and individual patients.

The disclosure includes a description of the quantitative relationships that describe the effect of mucin chain length and surface density on glycocalyx thickness. From the perspective of personalized medicine, mucin transcript levels, allele lengths, and surface levels are accessible quantities71,72. Combined with the scaling relations described herein, such metrics could be used to predict the likelihood of a mucin barrier and, thus, patient recalcitrance to immune therapies. Few transferable standards exist for benchmarking and reproducibly categorizing mucin surface levels in clinical or research samples across labs. The disclosure also provides as a potential standard the 1E7 cell line described herein, which allows titration of Muc1 levels with high reliability and reproducibility. The Examples also demonstrate that that StcE mucinase can be directly tethered to the immune cell surface via its X409 lectin domain to provide a performance benefit. The described approach of coupling the NK cells surface with StcE via its X409 lectin domain has some potential drawbacks: First, lectin-mediated affinity may not be sufficient for the prolonged conjugation necessary for in vivo applications. Second, the ligand for X409 on the NK surface is unknown and may be expressed at variable levels. Third, X409 lectin domain is unique to StcE, necessitating the development of alternative coupling strategies for other GE enzymes. To achieve tunable and universal display of GE enzymes on NK (and other type of immune cells) the disclosure demonstrates the GE-NK paradigm via a modular leucine-zipper coupling strategy. GE enzymes that could be coupled with this approach include other mucolytic agents, sialidase, and enzymes that digest other common structural elements in the glycocalyx, such as hyaluronic acid. The disclosure includes GE enzyme surface display encoding a fused membrane anchor. The disclosure includes use of StcE as well as more selective mucinsases, including but not necessarily limited to the above described BT4244 from Bacteroides thetaiotaomicron and M60-like protease AM0627 from Akkermansia muciniphila, which preferentially cleaves mucins with truncated, cancer-associated O-glycans⁷⁶.

### Example 8

This Example provides a description of the materials and methods used to produce the results described in Examples 1-6.

### Cell Lines

MCF10A cells were cultured in DMEM/F12 media (Thermo Fisher Scientific) supplemented with 5% horse serum (Thermo Fisher Scientific), 20 ng/mL EGF (Pepro Tech), 10 mg/ml insulin (Sigma), 500 ng/mL hydrocortisone (Sigma), 100 ng/mL cholera toxin (Sigma) and 1× penicillin/streptomycin (Thermo Fisher Scientific) at 37° C. in 5% CO₂. HEK293T cells was cultured in DMEM high glucose media (Thermo Fisher Scientific) supplemented with 10% fetal bovine serum (Thermo Fisher Scientific) and 1× penicillin/streptomycin (Thermo Fisher Scientific) at 37° C. in 5% CO₂. ZR-75-1 cells and T47D cells were cultured in RPMI media (Thermo Fisher Scientific) supplemented with 10% fetal bovine serum (Thermo Fisher Scientific) and 1× penicillin/streptomycin (Thermo Fisher Scientific) at 37° C. in 5% CO₂. SKOV-3 and SK-BR3 cells were cultured in McCoy's 5a Medium Modified (Thermo Fisher Scientific) supplemented with 10% fetal bovine serum and 1× penicillin/streptomycin. NK-92 cells (ATCC CRL-2407) were cultured in α-MEM without ribonucleosides media (Thermo Fisher Scientific) supplemented with 12.5% fetal bovine serum (Thermo Fisher Scientific), 12.5% horse serum (Thermo Fisher Scientific), 0.2 mM Myo-inositol (Sigma Aldrich), 0.1 mM 2-mercaptoethanol (Thermo Fisher Scientific), 0.02 mM folic acid (Millipore Sigma), 100 U/ml recombinant human IL-2 (Pepro Tech), and 1× penicillin/streptomycin (Thermo Fisher Scientific) at 37° C. in 5% CO₂.

### Antibodies and Reagents

The following antibodies were used: anti-Human Muc Clone HMPV (555925, BD Biosciences), anti-Human Muc Janelia Fluor 549 (NBP-2-47883JF549, Novus Biologicals), Recombinant anti-GNE antibody (ab189927, Abcam), anti-GCNT1 antibody (ab102665, Abcam), anti-Galectin-3 N-20 (19280, Santa Cruz), anti-Galectin-1 (ab108389, Abcam), mouse anti-3-Actin Clone C4 (47778, Santa Cruz), anti-ErbB2/HER2 Clone 3B5 (ab16901, Abcam), APC conjugated anti-hErbB2/HER2 (FABI129A, R&D systems), APC conjugated anti-Human perforin (Clone dG9, 30811, BioLegend), anti-ErbB2/HER2 Clone 3B5 (ab16901, Abcam), Anti-6× His-tag antibody (ab9108, Abcam). The following secondary antibodies were used: goat anti-mouse IgG DyLight 800 4×PEG conjugate (SA535521, Invitrogen), goat anti-mouse IgG DyLight 680 conjugate (35518, Invitrogen), goat anti-rabbit IgG Alexa Fluor 647 conjugate (A21245, Thermo Fisher Scientific), goat anti-mouse Alexa Fluor 647 conjugate (A28181, Invitrogen), and rabbit anti-goat IgG Alexa Fluor 647 conjugate (A21446, Invitrogen). Lectins used were: CF640R-conjugated PNA (#29063, Biotium), biotin-conjugated MAL-II (B-1265-1, Vector Laboratories). Biotinylated lectins were detected using NeutrAvidin Protein (DyLight 800 conjugated, 22853; DyLight 650 conjugated, 84607; both from Thermo Fisher Scientific). For tetracycline-inducible systems, doxycycline was used for induction (204734, Santa Cruz). For western blot, RIPA lysis buffer (89900, Thermo Fisher Scientific) and Halt Protease and Phosphatase Inhibitor Cocktail (78446, Thermo Fisher Scientific) were used. TD139 (28400, Cayman Chemical) was used to inhibit galectin-3 binding to Muc. For digesting N-glycan, PNGase F (P0704S, New England Biolabs) was used. For detecting dead cell population by NK-mediated killing assay, propidium iodide (P4170, Sigma) and Annexin V conjugated CF647 (#29003, Biotium) were used. For SAIM measurement, MemGlow dye (MemGlow 560: MG02-2; MemGlow 590: MG03-10; MemGlow 640: MG04-02, Cytoskeleton) or CellBrite Steady 550 (#30107-T, biotium) were used to label cell membrane. Myc-Tag (9B11) Mouse mAb conjugated with Alexa Fluor 647 (#2233S, Cell Signaling Technology) was used to detect surface level of a cognate leucine zipper level on NK-92 cells

### Cloning and Constructs

cDNAs for mOxGFP-tagged Muc1 were previously cloned into a PiggyBac expression system containing a tetracycline-inducible promoter (P_{CMV-min}-tetO₇) and a separate puromycin selectin cassette (pPB tetOn PuroR)^{43,77}. In this work, Muc1 cDNAs with approximately 42 native tandem repeats (TRs; Muc1 mO×GFP ACT) or either 0, 10, 21, and 42 perfect TRs of PDTRPAPGSTAPPAHGVTSA (SEQ ID NO:9) (0TR, 10TR, 21TR, 42TR) were used. Lentiviral plasmids for constitutive expression of human GCNT1, LGALS3 (galectin-3), HER2, and an anti-HER2 chimeric antigen receptor (CAR) were prepared by inserting the cDNAs in place of GFP in pLenti CMV GFP Hygro (Addgene #17446), using the BamHI and BsrGI restriction sites. The unmodified human cDNA for GCNT1 was generated by custom gene synthesis (General Biosystems). The human HER2 cDNA was amplified by PCR from the plasmid, HER2 WT (Addgene #16257), with primers that added 5′ BamHI and 3′ BsrGI restriction sites: 5′-TAATCTAGACCCAAGCTTGGGGCAG-3′ (SEQ ID NO:10) and 5′-GCTGTCGACGAGCTCGGTACCAAGCTT-3′ (SEQ ID NO:11). cDNA for human LGALS3 was amplified by PCR from a pET21a expression vector (gift of C. Bertozzi) with primers that added 5′ BamHI and 3′ BsrGI restriction sites: 5′-TAAGCGGATCCGAATTCTATGGCAGACAATT-3′ (SEQ ID NO:12) and 5′-TGCTTTGTACATTATATCATGGTATATGAAGC-3′ (SEQ ID NO:13). cDNA for a previously reported HER2-specific CAR⁶⁵—including a humanized FRP5 scFV, CD8a hinge, CD28 transmembrane domain, CD28 signaling domain, and CD3ζ signaling domain—was synthesized with a 2A peptide/mTagBFP2 expression reporter by custom gene synthesis (Twist Bioscience) (See Supplemental Table 1 for the complete sequence). A pLentiCRISPR V2 plasmid for knockout of GNE was created by inserting the oligos, 5′-CACCGACATCAAGTTCAAAGAACTC-3′ (SEQ ID NO:14) and 5′-AAACGAGTTCTTTGAACTTGATGTC-3′ (SEQ ID NO:15), into the BsmBI restriction site following standard protocols⁷⁸. Recombinant GFP nanobody cDNA (PDB #30GO E) was generated by custom gene synthesis (IDT) and cloned into the BamHI and HindIII restriction sites of pTP1112 (Addgene #104158). cDNA for Sialidase with IgK leader sequence, ALFA-Tag, type I transmembrane anchor (PDGFRP) from pDisplay system (Thermo Fisher) and superfold GFP (sfGFP) was synthesized by custom gene synthesis and inserted into a PiggyBac expression system containing a tetracycline-inducible promoter and a separate hygromycin selectin cassette.

### Generated Cell Lines

MCF10A cells stably expressing the rtTA-M2 tetracycline transactivator were prepared by lentiviral transduction using the pLV rtTA-NeoR plasmid as previously described.³⁸ For preparation of mucin-expressing cell lines, MCF10A rtTA-M2 cells were co-transfected with the PiggyBac hyperactive transposase and PiggyBac plasmids containing ITR-flanked expression cassettes (i.e. pPB tetOn PuroR plasmids) using the NEPA21 Electroporator (NEPAGENE) and subsequently selected with 1 μg/mL puromycin. The stable MCF10A line expressing native Muc mOxGFP ACT was clonally expanded by limiting dilution of single cells in 96-well plates. An individual clone, 1E7, was isolated that exhibited low leaky expression of Muc mOxGFP ACT and titratable Muc surface levels with doxycycline induction.

Progeny of the 1E7 clone overexpressing GCNT1, LGALS3, and HER2 were prepared by transduction with lentiviral particles generated using the respective pLenti CMV Hygro plasmids. Cell selection was with 200 μg/mL hygromycin. Control MCF10A rtTA-M2 cells overexpressing HER2 were similarly prepared. Knockout (KO) of GNE in the 1E7 clone was achieved by lentiviral transduction using the pLentiCRISPR V2 system and selection with 200 μg/mL hygromycin. The selected cells were clonally expanded and individual clones were screened for double knockout of GNE by Sanger sequencing of genomic DNA that was isolated using the Quick-DNA miniprep kit (Zymo, D3024).

KO of C1GALT1 in the 1E7 cells was achieved using the Alt-R CRISPR/Cas9 system (IDT) with homology-directed repair (HDR) templates that introduced an in-frame stop codon and an EcoRI restriction site for screening. CRISPR RNA (crRNA) and HDR template for C1GALT1 KO were: 5′-GTAAAGCAGGGCTACATGAG-3′ (SEQ ID NO:16) and 5′-CTTTGGGAGAAGATTTAAGCCTTATGTAAAGCAGGGCTACTAAGAATTCAGTGG AGGAGCAGGATATGTACTAAGCAAAGAAGCCTTGA-3′ (SEQ ID NO:17). CRISPR/Cas9 editing was conducted according to manufacturer's protocol for the Alt-R system. Briefly, 1E7 cells were transfected with precomplexed crRNA and ATTO550-tracrRNA, along with the HDR template, using electroporation. After 48 hours, transfected 1E7 cells were clonally expanded by limiting dilution in 96-well plates. To screen for successful knockouts, genomic regions encompassing the targeted CRISPR/Cas9 cut sites were amplified by PCR and analyzed by EcoRI restriction digest and Sanger sequencing. PCR primers for C1GALT were 5′-GGAGGATAATAGTTGTAATTCCAGTACCAAAAC-3′ (SEQ ID NO:18) and 5′-TCAAAACCTAGAGAAAAAGGCCAAACAC-3′ (SEQ ID NO: 19). PCR primers for GNE were 5′-AGTGGTTAAGGACTTGAAACTG-3′ (SEQ ID NO:20) and 5′-TCTACTAAGCGGCATCATTG-3′ (SEQ ID NO:21). Sequencing primer for C1GALT1 was 5′-ACACGTCAAAGCTACTTGG-3′ (SEQ ID NO:22) and sequencing primer for GNE was 5′-AGTGGTTAAGGACTTGAAACTG-3′ (SEQ ID NO:23). A NK-92 line expressing the HER2-CAR was prepared by transduction with lentiviral particles generated using the pLenti CMV Hygro plasmid. Cell selection was with 200 μg/mL hygromycin. Selected cells were sorted for high mTagBFP2 signal. A NK-92 line expressing a cognate leucine zipper (RR) was prepared by lentivirus transduction using pTwist SFFV Puro plasmids (Twist Bioscience). Cells were selected with 2 μg/mL Puromycin for 2 weeks or sorted with sfGFP expression to collect higher expression of RR-Zip on the cell surface of NK cells. (See Supplemental Table 2). NK-92 line stably expressing the rtTA-M2 tetracycline transactivator was prepared by lentiviral transduction using the pLV rtTA-NeoR plasmid as previously described.³⁸ For preparation of Sialidase-expressing cell lines, NK-92 rtTA-M2 cells were co-transfected with the PiggyBac hyperactive transposase and PiggyBac plasmids containing ITR-flanked expression cassettes (i.e. pPB tetOn Hygro plasmids) using the NEPA21 Electroporator (NEPAGENE) and subsequently selected with 200 μg/mL hygromycin. The stable NK-92 line expressing Sialidase-PDGFRβ-sfGFP was sorted with higher sfGFP expression.

### Western Blot Analysis

Cells were plated and induced with 1 g/mL doxycycline for 24 hours before lysis with RIPA buffer. Lysates were separated on NuPAGE 3-8% Tris-Acetate gels or NuPAGE 4-12% Bis Tris gels and transferred to low-fluorescence PVDF membranes (Milipore Sigma, IPFL07810). Primary antibodies were diluted at 1:500 and fluorophore-conjugated or biotinylated lectins were diluted to 2 g/mL in 5% BSA TBST and incubated overnight at 4° C. Secondary antibodies and Neutravidin-DyLight 800 were diluted at 1:1000 or 1 g/mL in 5% BSA TBST and incubated for 1 hour at room temperature. Blots were imaged on a ChemiDoc MP Imaging System (Bio-Rad)

### Flow Cytometric Analysis

Cells were plated, grown for 24 hours, and then induced with various concentrations of doxycycline for 24 hours. Adherent cells were detached by incubating with Accutase (Innovative Cell Technologies) at 37° C. for 10 minutes. GFP nanobody conjugated Alexa Fluor 647, Neutravidin conjugated DyLight 650, CF640R PNA, Alexa Fluor 647 HPA, and biotin MAL-II were diluted 1:200 in 0.5% BSA PBS and incubated with cells at 4° C. for 30 minutes for each stain. For analysis of Muc1 cell surface expression level, anti-Human Muc Clone HMPV was diluted 1:200 in 0.5% BSA PBS and incubated with cells at 4° C. (for 1 hour. Secondary labelling was with Alexa Fluor 647 conjugated goat anti-mouse, diluted 1:1000 in 0.5% BSA PBS and incubated with cells at 4° C. for 1 hour. For analysis of StcE mucinase binding to NK-92 cells, NK cells were incubated with StcE at 37° C. for 1 hour. Cells were thoroughly washed to remove StcE and coculture with target cells. Anti-6× His-tag antibody was diluted 1:100 in 0.5% BSA PBS and incubated with cells at 4° C. for 1 hour. Secondary labelling was with Alexa Fluor 647 goat anti-rabbit IgG, diluted 1:500 in 0.5% BSA PBS or FluoTag-X2 anti-AFLA conjugated with Alexa Fluor 647 was diluted 1:200 in 0.5% BSA PBS and incubated with cells at 4° C. for 1 hour. To measure cell surface level of leucine zipper or sialidase on NK-92 cells, Myc-tag antibody was diluted 1:10 in 0.5% BSA PBS and incubated with cells at 4° C. for 1 hour. To analyze fluorescent labelled cells, Attune N×T Flow cytometry (Thermo Fisher) was used.

### Immunofluorescence

MCF10A cells were plated and induced with 1, 100, and 1,000 ng/mL doxycycline for 24 hours. For endogenous galectins staining of cells, galectin antibodies were diluted 1:50 with 0.5% in 0.5% BSA PBS and incubated on 1E7 clone with 1,000 ng/mL of doxycycline at 4° C. for 1 hour after being fixed with 4% paraformaldehyde and permeabilized with 0.1% Triton X-100. For cell-surface galectin-3, TD139 (28400, Cayman Chemical) was diluted to 1, 10, 50 μM in MCF10A media and incubated on 1E7 clone with 1,000 ng/mL of doxycycline at 37° C. for 2 hours. TD139-treated cells were further incubated with anti-galectin-3 antibody at 4° C. for 30 minutes. For GCNT1 imaging, 1E7 clone cells were fixed with 4% paraformaldehyde and permeabilized with 0.1% Triton X-100 after induction of 1,000 ng/mL doxycycline. GCNT1 antibody was diluted 1:50 in 0.5% BSA PBS and incubated on samples at 4° C. for 1 hour. For NK-92 cell imaging, NK-92 cells were incubated with 100 nM StcE or StcE variants in NK cell culture media for 1 hour at 37° C. and washed thoroughly with phenol red-free DMEM/F12 supplemented with 5% horse serum, 20 ng/mL EGF, 10 mg/ml insulin, 500 ng/mL hydrocortisone, 100 ng/mL cholera toxin and 1× penicillin/streptomycin. 1E7 cells were plated to glass bottom dishes for 1 days and induced with 1,000 ng/mL doxycycline for 24 hours. StcE-treated NK cells were plated on 1E7 cells at 37° C. for 4 hours. For Hyaluronic acid imaging, cells were incubated with 2.5 pg/mL Hyaluronic Acid Binding Protein Biotinylated (385911, Millipore Sigma) at 4° C. for 1 hour. For other Muc1-GFP clones imaging, two other clones (2E4 and 2G9) were plated onto glass-bottom dishes and induced with 1, 100, 1,000 ng/mL of doxycycline (sc-204734, Santa Cruz) for 24 hours. Secondary antibodies were diluted 1:500 in 0.5% BSA PBS and incubated with cells at 4° C. for 1 hour. Samples were imaged on a LSM 800 confocal microscope using 10× (NA: 0.3 Air), 20× (NA: 0.8 Air), and 63× (NA: 1.4 Water) objectives (Zeiss).

### Preparation of Recombinant Human Galectins

Human LGALS1 and LGALS3 constructs in pET21 were recombinantly expressed in E. coli strain NiCo21 (DE3) (New England Biolabs). Transformed bacteria were grown in LB at 37° C. until an OD600 of 0.6-0.8 was reached. Expression was induced with 0.3 mM IPTG, and protein was produced overnight at 20° C. Cells were harvested by centrifugation at 3,000 g for 20 minutes and lysed by a pressurized homogenizer with cOmplete protease inhibitor Cocktail (Roche) and 1 mg/mL lysozyme (Sigma). The lysate was centrifuged at 20,000 rpm for 45 minutes at 4° C., and the supernatant was incubated with O-lactosyl Sepharose resin for 1 hour at 4° C. before loading into a gravity column. The protein was eluted with 0.1 M β-lactose (Santa Cruz Biotech) and 8 mM DTT (Sigma). The partially purified protein was polished on a HiPrep 16/60 Sephacryl S-100 HR (GE Healthcare Life Sciences) column equilibrated with 0.1 M β-lactose and 8 mM DTT. The final protein was then concentrated by using Amicon Ultra (3 kD MWCO for Galectin-1; 10 kD MWCO for Galectin-3) filters (Millipore Sigma). Conjugation of β-lactose to Sepharose 6B (Sigma) to synthesize the β-lactosyl Sepharose was as previously described⁷⁹.

StcE mucinase Expression and Purification

The cDNA for StcE-A35⁶⁰ was synthesized by custom gene synthesis (Twist Bioscience) and inserted into the pET28b expression vector (See Supplemental Table 2). StcE E447D was generated using Q5 Site-Directed Mutagenesis Kit (New England Biolabs) with primers 5′-TCAGTCATGACGTTGGTCATAATTATG-3′ (SEQ ID NO:24) and 5′-ACTCATTCCCCAATGTGG-3′ (SEQ ID NO:25). StcE-ΔX409 was also generated using the Q5 Site-Directed Mutagenesis Kit with primers 5′-TAACTCGAGCACCACCAC-3′ (SEQ ID NO:26) and 5′-ATTTACAGTATAGGTAAGTCCTTC-3′ (SEQ ID NO:27) (See Supplemental Table 2). The general design of StcE EE Zip is as follows^{62,80}. The 35-aa glycine/serine (GGGGS)₇ (SEQ ID NO:7) linker and leucine zipper (EE zip) construct was synthesized by custom gene synthesis (Twist Bioscience) and inserted into the pET28b StcE ΔX409 vector using NEBbuilder HiFi DNA Assembly (New England Biolabs) with primers 5′-TGAGATCCGGCTGCTAAC-3′ (SEQ ID NO:28) and 5′-ATTTACAGTATAGGTAAGTCCTTCTG-3′ (SEQ ID NO:29) (See Supplemental Table 2). A cognate leucine zipper (RR) with a IgK leader sequence, HA-tag, Myc-tag, type I transmembrane anchor (PDGFRP) from pDisplay system (Thermo Fisher) was synthesized by custom gene synthesis and inserted into pTwist SFFV Puro lentivirus vector (Twist Bioscience) (See Supplemental Table 2). An alternative leucine zipper (RR) construct with a IgK leader sequence, extracellular ALFA-Tag, type I transmembrane anchor (PDGFRP) from pDisplay system (Thermo Fisher) and superfold GFP (sfGFP) was synthesized by custom gene synthesis and inserted into pTwist SFFV lentivirus vector (Twist Bioscience) (See Supplemental Table 2). Cells were harvested by centrifugation at 3,000 g for 20 minutes, resuspended in lysis buffer (20 mM HEPES, 500 mM NaCl and 10 mM imidazole, pH 7.5) with cOmplete protease inhibitor Cocktail (Roche), and lysed by a pressurized homogenizer. StcE was purified by immobilized metal affinity chromatography (IMAC) on a GE AKTA Avant FPLC system. The lysate was loaded onto a HisTrap HP column (GE Healthcare Life Sciences), washed with 20 column volumes of wash buffer (20 mM HEPES, 500 mM NaCl and 20 mM imidazole, pH 7.5.), and eluted with a linear gradient of 20 mM to 250 mM imidazole in buffer (20 mM HEPES and 500 mM NaCl, pH 7.5.). The elution fractions containing target protein were collected and polished on a HiPrep 26/60 Sephacryl S-200 HR (GE Healthcare Life Sciences) column equilibrated with storage buffer (20 mM HEPES and 150 mM NaCl, pH 7.5). The final protein was then concentrated by using Amicon Ultra 30 kDa MWCO filters (Millipore Sigma)⁶¹.

### AM0627, BT4244, and Sialidase Expression and Purification

The cDNA for AM0627, BT4244, and Sialidase with 35-aa glycin/serine (GGGGS)₇ linker and leucine zipper (EE zip) construct were synthesized by custom gene synthesis (Twist Bioscience) and inserted into the pET28b expression vector (See Supplemental Table 2). Cells were harvested by centrifugation at 3,000 g for 20 minutes, resuspended in lysis buffer (20 mM HEPES, 500 mM NaCl and 10 mM imidazole, pH 7.5) with cOmplete protease inhibitor Cocktail (Roche), and lysed by a pressurized homogenizer. Mucinases was purified by immobilized metal affinity chromatography (IMAC) on a GE AKTA Avant FPLC system. The lysate was loaded onto a HisTrap HP column (GE Healthcare Life Sciences), washed with 20 column volumes of wash buffer (20 mM HEPES, 500 mM NaCl and 20 mM imidazole, pH 7.5.), and eluted with a linear gradient of 20 mM to 250 mM imidazole in buffer (20 mM HEPES and 500 mM NaCl, pH 7.5.). The elution fractions containing target protein were collected. The final protein was then concentrated by using Amicon Ultra 30 kDa MWCO filters (Millipore Sigma).

### GFP Nanobody Preparation and Conjugation

Recombinant GFP nanobody (PDB #3OGO E) was prepared in chemically competent NiCo21 (DE3) E. coli (NEB) using the pTP1112 plasmid. Transformed bacteria were grown in LB at 37° C. until an OD600 of 0.6 was reached. The cultures were induced with 0.5 mM IPTG overnight at 24° C., harvested, resuspended in B-PER (Thermo Fisher Scientific) and vortexed for cell lysis. The lysates were cleared by centrifugation at 10,000 g for 20 minutes at 4° C. The His-tagged nanobody was purified by IMAC according to standard protocols. Briefly, supernatant diluted into 1×Ni-NTA binding buffer was bound to equilibrated Ni-NTA resin (Qiagen, 30210) for 20 minutes at 4° C., with end-over-end mixing. The resin was added to a spin column, washed thoroughly, and incubated with the Ni-NTA elution buffer for 20 minutes at 4° C., mixing end-over-end. Eluted protein was exchanged into storage buffer (pH 7.4 PBS) using Zeba 7K MWCO desalting columns or by overnight dialysis with 10K MWCO Snakeskin dialysis tubing. Eluted proteins were then sterile filtered and snap-frozen for long-term storage at −80° C. Nanobody-containing constructs were mixed with 0.1% w/v sodium azide prior to snap-freezing. For fluorescent GFP nanobody, purified GFP nanobody was labeled with Alexa Fluor 647 NHS Ester (Thermo Fisher Scientific) per the manufacturer's protocol.

### NK Cell Cytotoxicity Assay

Target MCF10A cells were fluorescently labeled by incubation with 1 M CellTracker Green CMFDA Dye (Invitrogen) in MCF10A growth media for 10 minutes. 3×10⁴ labeled target cells were suspended with varying ratios of NK-92 effector cells in 200 μL of growth media of target cell line in absent of IL-2 and cocultured in an ultra-low attachment U-bottom 96-well plate (Corning) for 4 hours at 37° C. in 5% C02. For all StcE-treated NK cell experiment, unless otherwise indicated, NK cells were incubated with 100 nM StcE at 37° C. for 1 hour. Cells were thoroughly washed to remove StcE and coculture with target cells. After 4 hours, propidium iodide (PI; 20 μg/mL, Sigma) was added to each well for 10 minutes and washed with 0.5% BSA PBS. For different Muc surface level on ZR-75-1, anti-Human Muc Janelia Fluor 549 was diluted 1:200 in 0.5% BSA PBS and incubated with ZR-75-1 cells at 4° C. for 1 hour and sorted the cells with defined standardized gates for low, moderate, and high Muc expression by FACS (Sony MA900 Cell Sorter). Sorted target cells were cocultured with varying ratios of NK-92 effector cells for 4 hours. Annexin V conjugated with CF647 (0.5 g/mL, Biotium) was diluted in HEPES-buffered saline containing 2.5 mM calcium chloride and added to each well for 15 minutes. NK cell cytotoxicity was evaluated by flow cytometry as previously described^{16,81}. At least 1×10⁴ target cells were analyzed after electronic gating on CellTracker Green. Percent cytotoxicity was calculated as 100×(experimental % dead−spontaneous % dead)/(100−spontaneous % dead), where experimental % dead was the percentage of PI positive target cells in NK cocultures and spontaneous % dead was the percentage of PI positive control target cells cultured in the absence of effector cells.

Fluorescence Recovery after Photobleaching

FRAP experiments were performed by using a Zeiss i880 confocal microscope with 63×N.A. 1.4 Oil immersion lens. Five pre-bleached images were firstly taken and photobleaching was performed with 488 nm laser for about 10 seconds to bleach a target area of 2.55 μm×2.55 μm. 1E7 clone (n=11) and 1E7 GCNT1 overexpression (n=10) were used to measure mobile fraction of Muc1-GFP. 10 μM recombinant human galectin-1 was incubated with 1E7 GCNT1 cells for 10 minutes at 37° C. (n=9). Intensity profiles were corrected by normalizing to the time-course decay of GFP signal in non-bleached area by using MATLAB code from Advanced Imaging Center (https://www.mathworks.com/matlabcentral/fileexchange/47327-frap-zip)

### Scanning Angle Interference Microscopy

### Sample Preparation

Silicon wafers with ˜1900 nm thermal oxide (Addison Engineering) were diced into 7 mm by 7 mm chips, and the oxide layer thickness for each chip was measured with a FilMetrics F50-EXR. Prior to use, the chips were then cleaned with 1:1 methanol and hydrochloric acid for 15 minutes, followed by 1 minutes in a plasma cleaner (Harrick Plasma, PDC-001). The chips were incubated with 4% (v/v) (3-mercaptopropyl)trimethoxysilane in absolute ethanol for 30 minutes at room temperature. After washing with absolute ethanol, the chips were subsequently incubated with 4 mM 4-maleimidobutyric acid N-hydroxysuccinimide ester in absolute ethanol for 30 minutes and rinsed with PBS. 50 μg/mL human plasma fibronectin in PBS was incubated with the functionalized chips overnight at 4° C. for conjugation. Cells were seeded onto the chips at 2×10⁴ cm⁻² in full culture medium with varying doxycycline concentrations and incubated for 24 hours. Cells expressing different length of Muc1 were labelled with GFP nanobody conjugated to Alexa Fluor 647 in MCF10A growth media for 10 minutes at 37° C. Cells were labelled with MemGlow dye or CellBrite Steady 550 in serum-free, phenol red-free DMEM/F12 for 10 minutes at 37° C. Sample were then rinsed 3× in PBS, inverted into a 35 mm glass-bottom imaging dish containing imaging buffer (MemGlow dye or CellBrite dye: serum-free, phenol red-free DMEM/F12, Alexa Fluor 647-conjugated GFP nanobody: phenol red-free DMEM/F12 supplemented with 5% horse serum, 20 ng/mL EGF, 10 mg/ml insulin, 500 ng/mL hydrocortisone, 100 ng/mL cholera toxin and 1× penicillin/streptomycin) and imaged at 37° C. For NK-92 cell, the cleaned chips were incubated with 2.5% (v/v) (3-mercaptopropyl)trimethoxysilane, 4.5% (v/v) deionized water and 0.9% (v/v) acetic acid) in methanol overnight at 4° C. After washing with PBS, the chips were reacted with 0.1 mg/mL of maleimide-activated neutravidin protein (31007, Thermo Scientific) with 50 μg/mL fibronectin for 1 hour at room temperature and rinsed with PBS. A biotin-conjugated MAL-II were diluted 1:200 in PBS and incubated with the chips for 1 hour at room temperature. NK-92 cells were seeded onto the chips at 2×10⁴ cm⁻² in full culture medium and incubated for 1 hour⁸².

### Optical Setup

Scanning angle interference microscopy (SAIM) was conducted on a custom circle-scanning microscope.⁴⁰ The core of the setup was an inverted fluorescence microscope (Ti-E, Nikon). The excitation lasers (488 nm, Coherent; 560 nm, MPB Communications Inc.; and 642 nm, MPB Communications Inc.) were combined into a colinear beam by a series of dichroic mirrors (Chroma). The combined output beam was attenuated and shuttered by an AOTF (AA Opto-Electronic). The beam was directed onto a pair of galvanometer scanning mirrors (Cambridge Technology). The image of the laser on the scanning mirrors was magnified and relayed to the sample by two 4 f lens systems, a beam expanding telescope and a scan lens/objective lens combination. The beam expander was formed by f=30 mm and f=300 mm achromatic lenses with a m=1 zero-order vortex half-wave plate positioned between them and positioned 2 f from the 300 mm achromatic scan lens (Thorlabs). SAIM experiments were performed with a 60×N.A. 1.27 water immersion objective (Nikon). Fluorescence emission was collected with a quad band filter cube and single band filters (TRF89901-EMv2, Chroma) mounted in a motorized filter wheel (Sutter). Images were acquired with a Zyla 4.2 sCMOS (Andor) camera or an iXon 888 EMCCD (Andor) using the microscope's 1.5× magnifier for a total magnification of 90×. The open-source software Micro-Manager was used for camera and filter wheel control and image acquisition. The circle-scanning galvometers were operated in an autonomous fashion using a custom-designed 16-bit PIC microcontroller, which has been described previously⁴⁰.

### Image Acquisition and Analysis

For SAIM, 32 images were acquired at varying incidence angles for the circle-scanned excitation beam. During a typical image acquisition sequence, changes in the scanned incidence angle were triggered by a TTL signal from the camera to the microcontroller. The incidence angles were evenly spaced from 5 to 43.75 degrees with respect to the wafer normal in the imaging media. To obtain the reconstructed height topography of the samples, the raw image intensities, Iⱼ, at each incidence angle, θⱼ, were fit pixelwise by nonlinear least-squares to an optical model:

$${I}_{j} = A * f ( {θ}_{j} , H ) + B \qquad ( 1 )$$

where H is the unknown sample height and A and B are additional fit parameters. The vortex half-wave plate in the optical setup maintained the s-polarization of the circle-scanned excitation laser. For s-polarized monochromatic excitation of wavelength, λ, the probability of excitation, ƒ(θⱼ, H), for the system is given by:

$$f ( {θ}_{j} , H ) = 1 + 2 Re \{ {r}^{T E} \} cos ϕ - 2 Im \{ {r}^{T E} \} sin ϕ + Re {\{ {r}^{T E} \}}^{2} + Im {\{ {r}^{T E} \}}^{2} \qquad ( 2 )$$

where the phase shift, ϕ, and the reflection coefficient for the transverse electric wave, r^{TE}, are given by:

$$ϕ ( H ) = \frac{4 π}{λ} ( {n}_{b} H cos {θ}_{b} ) \qquad ( 3 )$$

$$rTE = \frac{( {m}_{11}^{TE} + {m}_{12}^{TE} {p}_{Si} ) {p}_{2} - ( {m}_{21}^{TE} + {m}_{22}^{TE} {p}_{0} )}{( {m}_{11}^{TE} + {m}_{12}^{TE} {p}_{Si} ) {p}_{2} + ( {m}_{21}^{TE} + {m}_{22}^{TE} {p}_{0} )} \qquad ( 4 )$$

$${M}^{TE} = ( \begin{matrix}{m}_{11}^{T E} & {m}_{12}^{T E} \\ {m}_{21}^{T E} & {m}_{22}^{T E}\end{matrix} ) = ( \begin{matrix}cos ( {k}_{o x} {d}_{o x} cos {θ}_{o x} ) & - \frac{i}{{p}_{1}} sin ( {k}_{o x} {d}_{o x} cos {θ}_{o x} ) \\ - i {p}_{1} sin ( {k}_{o x} {d}_{o x} cos {θ}_{o x} ) & cos ( {k}_{o x} {d}_{o x} cos {θ}_{o x} )\end{matrix} ) \qquad ( 5 )$$

$${p}_{0} = {n}_{S i} cos {θ}_{S i} , {p}_{1} = {n}_{o x} cos {θ}_{ox} , {p}_{2} = {n}_{b} cos {θ}_{b} \qquad ( 6 )$$

$${k}_{i} = \frac{2 π {n}_{i}}{λ} , {θ}_{o x} = {sin}^{- 1} \frac{n sin {θ}_{b}}{{n}_{o x}} , {θ}_{S i} = {sin}^{- 1} \frac{n sin {θ}_{o x}}{{n}_{S i}} \qquad ( 7 )$$

where kᵢ is the wavenumber in material i; n_{Si}, nₒₓ and n_{b} are the refractive index of the silicon, silicon oxide and sample, respectively; θ_{Si}, θₒₓ and θ_{b} are the angles of incidence in the silicon, silicon oxide and sample, respectively; and dₒₓ is the thickness of the silicon oxide layer. The angles of incidence in silicon oxide and silicon were calculated according to Snell's Law. To quantify the glycocalyx thickness of cells, the average height above the silicon substrate was calculated for a 100×100 pixel subregion in each cell. The glycocalyx thickness was reported as the height of the localized GFP nanobody, MemGlow dye or CellBrite dye signal minus the height of the fluorescently labeled fibronectin layer on the silicon substrate. Packages for fitting SAIM image sequences with the above model have been implemented in C++ and Julia.

This reference listing pertains to the Examples above and is not an indication that any reference is material to patentability.
1. Ghasempour, S. & Freeman, S. A. The glycocalyx and immune evasion in cancer. FEBS Journal 1-11 (2021) doi:10.1111/febs.16236.
2. Kim, Y. J. & Varki, A. Perspectives on the significance of altered glycosylation of glycoproteins in cancer. Glycoconjugate Journal 14, 569-576 (1997).
3. Ladenson, R. P., Schwartz, S. O. & Ivy, A. C. Incidence of the blood groups and the secretor factor in patients with pernicious anemia and stomach carcinoma. The American Journal of Medical Sciences 217, 194-197 (1949).
4. Hakomori, S. I. & Murakami, W. T. Glycolipids of hamster fibroblasts and derived malignant-transformed cell lines. Proceedings of the National Academy of Sciences of the United States of America 59, 1-8 (1967).
5. Zhou, J. Y. & Cobb, B. A. Glycans in Immunologic Health and Disease. Annual Review of Immunology 39, 511-536 (2021).
6. RodrÍguez, E., Schetters, S. T. T. & Kooyk, Y. van. The tumour glyco-code as a novel immune checkpoint for immunotherapy. Nature Reviews Immunology 18, 204-211 (2018).
7. Wall, S. Van De, Santegoets, K. C. M., Houtum, E. J. H. Van, Bull, C. & Adema, G. J. Sialoglycans and Siglecs Can Shape the Tumor Immune Microenvironment. Trends in Immunology 41, 274-285 (2020).
8. Mereiter, S., Balmana, M., Coampos, D., Gomes, J. & Reis, C. A. Glycosylation in the Era of Cancer-Targeted Therapy: Where Are We Heading?Cancer Cell 36, 6-16 (2019).
9. Burchell, J. M., Beatson, R., Graham, R., Taylor-papadimitriou, J. & Tajadura-ortega, V. O-linked mucin-type glycosylation in breast cancer. Biochemical Society Transactions 46, 779-788 (2018).
10. Gupta, R., Leon, F., Rauth, S., Batra, S. K. & Ponnusamy, M. P. A Systematic Review on the Implications of O-linked Glycan Branching and Truncating Enzymes on Cancer. Cells 9, 446 (2020).
11. Hanisch, F. O-Glycosylation of the Mucin Type. Biological Chemistry 382, 143-149 (2001).
12. Nason, R. et al. Display of the human mucinome with defined O-glycans by gene engineered cells. Nature Communications 1-16 (2021) doi:10.1038/s41467-021-24366-4.
13. Narimatsu, Y. et al. An Atlas of Human Glycosylation Pathways Enables Display of the Human Glycome by Gene Engineered Cells. Molecular Cell 75, 394-407.e5 (2019).
14. Rømer, T. B. et al. Mapping of truncated O-glycans in cancers of epithelial and non-epithelial origin. British Journal of Cancer (2021) doi:10.1038/s41416-021-01530-7.
15. Perdicchio, M., Ilarregui, J. M., Verstege, M. I., Cornelissen, L. A. M. & Schetters, S. T. T. Sialic acid-modified antigens impose tolerance via inhibition of T-cell proliferation and de novo induction of regulatory T cells. Proceedings of the National Academy of Sciences of the United States of America 113, 3329-3334 (2016).
16. Hudak, J. E., Canham, S. M. & Bertozzi, C. R. Glycocalyx engineering reveals a Siglec-based mechanism for NK cell immunoevasion. Nature Chemical Biology 10, 69-75 (2014).
17. Dusosw, S. A. et al. Glioblastomas exploit truncated O-linked glycans for local and distant immune modulation via the macrophage galactose-type lectin. Proceedings of the National Academy of Sciences of the United States of America 117, 3693-3703 (2020).
18. Beatson, R. et al. The mucin MUC1 modulates the tumor immunological microenvironment through engagement of the lectin. Nature Immunology 17, 1273-1281 (2016).
19. Rodriguez, E. et al. Sialic acids in pancreatic cancer cells drive tumour-associated macrophage differentiation via the Siglec receptors Siglec-7 and Siglec-9. Nature Communications 12, 1270 (2021).
20. Liu, F. & Rabinovich, G. A. Galectins as modulators of tumour progression. Nature Reviews Cancer 5, 29-41 (2005).
21. Perillo, N. L., Pace, K. E., Seilhamer, J. J. & Baum, L. G. Apoptosis of T cells mediated by galectin-1. Nature 378, 736-739 (1995).
22. Compagno, D. et al. Galectins as Checkpoints of the Immune System in Cancers, Their Clinical Relevance, and Implication in Clinical Trials. Biomolecules 10, 750 (2020).
23. Kuo, J. C. H., Gandhi, J. G., Zia, R. N. & Paszek, M. J. Physical biology of the cancer cell glycocalyx. Nature Physics 14, 658-669 (2018).
24. Paszek, M. J. et al. The cancer glycocalyx mechanically primes integrin-mediated growth and survival. Nature 511, 319-325 (2014).
25. Hollingsworth, M. A. & Swanson, B. J. Mucins in cancer: protection and control of the cell surface. Nature Reviews Cancer 4, 45-60 (2004).
26. Suzuki, Y., Sutoh, M., Hatakeyama, S. & Mori, K. MUC1 carrying core 2 O-glycans functions as a molecular shield against NK cell attack, promoting bladder tumor metastasis. International journal of oncology 40, 1831-1838 (2012).
27. Madsen, C. B. et al. Glycan Elongation Beyond the Mucin Associated Tn Antigen Protects Tumor Cells from Immune-Mediated Killing. PLoS ONE 8, e72413 (2013).
28. Kramer, J. R., Onoa, B., Bustamante, C. & Bertozzi, C. R. Chemically tunable mucin chimeras assembled on living cells. Proceedings of the National Academy of Sciences of the United States of America 112, 12574-12579 (2015).
29. Kuo, J. C. & Paszek, M. J. Glycocalyx Curving the Membrane: Forces Emerging from the Cell Exterior. Annual Review of Cell and Developmental Biology 37, 257-285 (2021).
30. Shimasaki, N., Jain, A. & Campana, D. NK cells for cancer immunotherapy. Nature Reviews Drug Discovery 19, 200-218 (2020).
31. Liu, E. et al. Use of CAR-Transduced Natural Killer Cells in CD19-Positive Lymphoid Tumors. The New England Journal of Medicine 382, 545-553 (2020).
32. Xiao, L. et al. Adoptive Transfer of NKG2D CAR mRNA-Engineered Natural Killer Cells in Colorectal Cancer Patients. Molecular Therapy 27, 1114-1125 (2019).
33. Tang, X. et al. First-in-man clinical trial of CAR NK-92 cells: safety test of CD33-CAR NK-92 cells in patients with relapsed and refractory acute myeloid leukemia. American Journal of Cancer Research 8, 1083-1089 (2018).
34. Okamoto, T. et al. Core2 O-glycan-expressing prostate cancer cells are resistant to NK cell immunity. Molecular Medicine Reports 7, 359-364 (2013).
35. Tsuboi, S. et al. A novel strategy for evasion of NK cell immunity by tumours expressing core2 O-glycans. The EMBO Journal 30, 3173-3185 (2011).
36. Son, S. et al. Molecular height measurement by cell surface optical profilometry (CSOP). Proceedings of the National Academy of Sciences of the United States of America 117, 14209-14219 (2020).
37. Möckl, L. et al. Quantitative Super-Resolution Microscopy of the Mammalian Glycocalyx. Developmental Cell 50, 57-72.e6 (2019).
38. Paszek, M. J. et al. Scanning angle interference microscopy reveals cell dynamics at the nanoscale. Nature Methods 9, 825-827 (2012).
39. Carbone, C. B., Vale, R. D. & Stuurman, N. An acquisition and analysis pipeline for scanning angle interference microscopy. Nature Methods 13, 897-898 (2016).
40. Colville, M. J., Park, S., Zipfel, W. R. & Paszek, M. J. High-speed device synchronization in optical microscopy with an open-source hardware control platform. Scientific Reports 9, 1-13 (2019).
41. Ajo-Franklin, C. M., Ganesan, P. V. & Boxer, S. G. Variable incidence angle fluorescence interference contrast microscopy for Z-imaging single objects. Biophysical Journal 89, 2759-2769 (2005).
42. Lambacher, A. & Fromherz, P. Fluorescence interference-contrast microscopy on oxidized silicon using a monomolecular dye layer. Applied physics A 216, 207-216 (1996).
43. Shurer, C. R. et al. Physical Principles of Membrane Shape Regulation by the Glycocalyx. Cell 177, 1757-1770.e21 (2019).
44. Button, B. et al. Periciliary Brush Promotes the Lung Health by Separating the Mucus Layer from Airway Epithelia. Science 24, 937-941 (2012).
45. Song, D., Cahn, D. & Duncan, G. A. Mucin biopolymers and their barrier function at airway surfaces. Langmuir 36, 12773-12783 (2020).
46. Milner, S. T. Polymer Brushes. Science 251, 905-914 (1991).
47. O'Connor, J. C., Julian, J., Lim, S. D. & Carson, D. D. MUC1 expression in human prostate cancer cell lines and primary tumors. Prostate Cancer and Prostatic Diseases 8, 36-44 (2005).
48. Walsh, M. D., Luckie, S. M., Cummings, M. C., Antalis, T. M. & McGuckin, M. A.
Heterogeneity of MUC1 expression by human breast carcinoma cell lines in vivo and in vitro. Breast Cancer Research and Treatment 58, 255-266 (1999).
49. Gandhi, J. G., Koch, D. L. & Paszek, M. J. Equilibrium Modeling of the Mechanics and Structure of the Cancer Glycocalyx. Biophysical Journal 116, 694-708 (2019)
50. Gennes, P. G. de. Polymers at an interface; a simplified view. Advances in Colloid and Interface Science 27, 189-209 (1987).
51. Arai, S. et al. Infusion of the allogeneic cell line NK-92 in patients with advanced renal cell cancer or melanoma: A phase I trial. Cytotherapy 10, 625-632 (2008).
52. Williams, B. A. et al. A phase I trial of NK-92 cells for refractory hematological malignancies relapsing after autologous hematopoietic cell transplantation shows safety and evidence of efficacy. Oncotarget 8, 89256-89268 (2017).
53. Rosenstock, P., Horstkorte, R., Gnanapragassam, V. S., Harth, J. & Kielstein, H. Siglec-7 expression is reduced on a natural killer (NK) cell subset of obese humans. Immunologic Research 65, 1017-1024 (2017).
54. Paturej, J., Sheiko, S. S., Panyukov, S. & Rubinstein, M. Molecular structure of bottlebrush polymers in melts. Science Advances 2, (2016).
55. Sarapas, J. M., Martin, T. B., Chremos, A., Douglas, J. F. & Beers, K. L. Bottlebrush polymers in the melt and polyelectrolytes in solution share common structural features. Proceedings of the National Academy of Sciences of the United States of America 117, 5168-5175 (2020).
56. Argüeso, P. et al. Association of cell surface mucins with galectin-3 contributes to the ocular surface epithelial barrier. Journal of Biological Chemistry 284, 23037-23045 (2009).
57. Earl, L. A., Bi, S. & Baum, L. G. N- and O-Glycans Modulate Galectin-1 Binding, CD45 Signaling, and T Cell Death. The Journal of Biological Chemistry 285, 2232-2244 (2010).
58. Yu, L. et al. Galectin-3 Interaction with Thomsen-Friedenreich Disaccharide on Cancer-associated MUC1 Causes Increased Cancer Cell Endothelial Adhesion. Journal of Biological Chemistry 282, 773-781 (2007).
59. Zhao, Q. et al. Circulating Galectin-3 Promotes Metastasis by Modifying MUC1 Localization on Cancer Cell Surface. Cancer Research 69, 6799-6807 (2009).
60. Yu, A. C. Y., Worrall, L. J. & Strynadka, N. C. J. Structural Insight into the Bacterial Mucinase StcE Essential to Adhesion and Immune Evasion during Enterohemorrhagic E. coli Infection. Structure 20, 707-717 (2012).
61. Malaker, S. A. et al. The mucin-selective protease StcE enables molecular and functional analysis of human cancer-associated mucins. Proceedings of the National Academy of Sciences of the United States of America 116, 7278-7287 (2019).
62. Cho, J. H., Collins, J. J. & Wong, W. W. Universal Chimeric Antigen Receptors for Multiplexed and Logical Control of T Cell Responses. Cell 173, 1426-1438.e11 (2018).
63. Zhang, C. et al. Chimeric antigen an off-the-shelf Cellular therapeutic for targeted elimination of Cancer Cells and induction of protective antitumor immunity. Frontiers in Immunology 8, (2017).
64. Zhang, C. et al. ErbB2/Her2-specific NK cells for adoptive immunotherapy of glioblastoma. Journal for ImmunoTherapy of Cancer 2(Suppl 3), P41 (2014).
65. Schönfeld, K. et al. Selective Inhibition of Tumor Growth by Clonal NK Cells Expressing an ErbB2/HER2-Specific Chimeric Antigen Receptor. Molecular Therapy 23, 330-338 (2015).
66. Wang, L. et al. Monoclonal antibody targeting MUC1 and increasing sensitivity to docetaxel as a novel strategy in treating human epithelial ovarian cancer. Cancer Letters 300, 122-133 (2011).
67. Cipollone, J. A. et al. The anti-adhesive mucin podocalyxin may help initiate the transperitoneal metastasis of high grade serous ovarian carcinoma. Clinical and Experimental Metastasis 29, 239-252 (2012).
68. Singh, R. et al. Target-specific cytotoxic activity of recombinant immunotoxin scFv(MUC1)-ETA on breast carcinoma cells and primary breast tumors. Molecular Cancer Therapeutics 6, 562-569 (2007).
69. Giannakouros, P., Comamala, M., Matte, I., Rancourt, C. & Piché, A. MUC16 mucin (CA125) regulates the formation of multicellular aggregates by altering β-catenin signaling. American journal of cancer research 5, 219-30 (2015).
70. Coelho, R. et al. Mucins and truncated O-glycans unveil phenotypic discrepancies between serous ovarian cancer cell lines and primary tumours. International Journal of Molecular Sciences 19, 1-13 (2018).
71. Tyanova, S. et al. Proteomic maps of breast cancer subtypes. Nature Communications 7, 1-11 (2016).
72. Ashley, E. A. Towards precision medicine. Nature Reviews Genetics 17, 507-522 (2016).
73. Bell, G. I., Dembo, M. & Bongrand, P. Cell Adhesion. Competition Between Nonspecific Repulsion and Specific Bonding. Biophysical Journal 45, 1051-1064 (1984).
74. Waldman, A. D., Fritz, J. M. & Lenardo, M. J. A guide to cancer immunotherapy: from T cell basic science to clinical practice. Nature Reviews Immunology 20, 651-668 (2020).
75. Xiao, H., Woods, E. C., Vukojicic, P. & Bertozzi, C. R. Precision glycocalyx editing as a strategy for cancer immunotherapy. Proceedings of the National Academy of Sciences of the United States of America 113, 10304-10309 (2016).
76. Shon, D. J. et al. An enzymatic toolkit for selective proteolysis, detection, and visualization of mucin-domain glycoproteins. Proceedings of the National Academy of Sciences of the United States of America 117, 21299-21307 (2020).
77. Pan, H., Colville, M. J., Supekar, N. T., Azadi, P. & Paszek, M. J. Sequence-Specific Mucins for Glycocalyx Engineering. ACS Synthetic Biology 8, 2315-2326 (2019).
78. Sanjana, N. E., Shalem, O. & Zhang, F. Improved vectors and genome-wide libraries for CRISPR screening iPipet: sample handling using a tablet. Nature Methods 11, 783-784 (2014).
79. Pace, K. E., Hahn, H. P. & Baum, L. G. Preparation of Recombinant Human Galectin-1 and Use in T-Cell Death Assays. Methods in Enzymology 363, 499-518 (2003).
80. Wong, W. W. & Cho, J. H. Methods and compositions relating to chimeric antigen receptors. (2018).
81. Bryceson, Y. et al. Functional Analysis of Human NK Cells by Flow Cytometry. Natural Killer Cell Protocols 612, 335-352 (2010).
82. Kim, D. H. et al. Direct visualization of single-molecule membrane protein interactions in living cells. PLoS Biology 16, 1-23 (2018).
83. Wels, W., Schonfeld, K., Tonn, T., Grez, M. & Zhang, C. Car-expressing NK-92 cells as cell therapeutic agents. US patent (U.S. Pat. No. 9,920,132B2) (2018).

### Example 8

This Example expands on the foregoing Examples and description. Example 8 is illustrated by FIGS. 12, 13, and 14. FIG. 12 provides a modular strategy for producing NK cells that are modified to comprise a GE enzyme using a described leucine zipper approach. FIG. 12 therefore provides a description of Enzyme-Zip NK cells and characterization of the cells, as described in the description of FIG. 12 above. FIG. 13 illustrates a combinatorial approach for producing GE-NK, as described in the description of FIG. 13 above. FIG. 14 illustrates genetic expression of sialidase-transmembrane fusion proteins in NK cells, as described in the description of FIG. 14 above.

SUPPLEMENTAL TABLE 1
Complete DNA sequence for HER2-specific CAR
expression HER2-specific CAR includes FRP5
scFv, CD8a Hinge, CD28 TM, and CD3ζ. mTagBFP2
sequence is followed by P2A sequence after CD3ζ.

| FRP5 scFV | CAAGTTCAACTTCAACAAAGTGGGCCAGAACTCAAGAAACCAGGGG |
|---|---|
| | AAACTGTGAAAATTTCATGTAAAGCTTCAGGATATCCATTTACAAA |
| | TTATGGGATGAATTGGGTCAAGCAAGCTCCCGGACAAGGTCTCAAA |
| | TGGATGGGGTGGATAAATACGTCCACAGGTGAATCCACATTTGCGG |
| | ATGACTTTAAAGGACGGTTTGATTTTAGTCTCGAGACTTCTGCTAA |
| | CACTGCTTATCTCCAAATTAATAATCTGAAATCTGAAGATTCCGCA |
| | ACATATTTCTGTGCACGGTGGGAAGTCTATCATGGTTATGTCCCTT |
| | ATTGGGGACAAGGTACAACTGTAACTGTATCTTCCGGTGGGGGGGG |
| | AAGTGGAGGAGGCGGCAGTGGTGGTGGCGGGTCTGATATACAACTC |
| | ACGCAATCTCATAAATTCCTTTCTACGTCCGTTGGAGATAGAGTCA |
| | GCATTACGTGTAAGGCTTCCCAAGATGTCTATAATGCAGTCGCATG |
| | GTACCAACAGAAACCAGGGCAAAGTCCTAAATTGCTTATATATTCT |
| | GCTTCTTCCAGGTATACTGGTGTCCCTTCCCGCTTTACGGGGTCCG |
| | GATCTGGGCCTGATTTTACGTTTACTATTTCATCCGTCCAAGCGGA |
| | AGATCTCGCTGTCTATTTCTGTCAACAGCATTTTCGTACCCCTTTT |
| | ACATTTGGAAGTGGAACGAAACTCGAGATAAAA (SEQ ID NO: |
| | 30) |
| CD8a Hinge | GCACTTAGTAATTCCATTATGTATTTTAGCCATTTTGTCCCAGTCT |
| | TCCTTCCAGCAAAACCTACTACAACACCAGCGCCACGCCCACCAAC |
| | GCCTGCACCTACGATAGCATCTCAACCACTCAGTCTCCGACCAGAA |
| | GCGTCTAGGCCAGCGGCCGGCGGCGCGGTCCATACAAGAGGGCTTG |
| | AT (SEQ ID NO: 31) |
| CD28 TM | AAACCATTTTGGGTACTCGTGGTTGTAGGTGGTGTTTTGGCTTGCT |
| | ATTCCCTGCTTGTTACAGTCGCGTTTATTATTTTCTGGGTTCGTTC |
| | TAAAAGATCTCGCCTTTTGCATTCTGATTATATGAATATGACTCCC |
| | CGCAGACCTGGGCCGACTAGAAAACATTATCAACCGTACGCTCCCC |
| | CGCGGGATTTTGCTGCATATAGAAGC (SEQ ID NO: 32) |
| CD3ζ | CGGGTCAAATTTTCACGTTCCGCTGATGCACCCGCATATCAACAAG |
| | GACAAAATCAACTTTATAATGAACTCAATCTCGGGCGGCGCGAAGA |
| | GTATGATGTTCTCGATAAAAGGCGGGGCCGAGATCCCGAAATGGGT |
| | GGGAAACCGCGGAGAAAGAATCCTCAAGAGGGGCTTTACAATGAGT |
| | TGCAAAAGGATAAAATGGCGGAAGCTTATTCTGAAATTGGGATGAA |
| | AGGAGAACGCCGCAGAGGGAAAGGTCATGATGGGCTCTATCAAGGG |
| | CTCAGCACGGCAACTAAAGATACTTATGATGCTCTTCATATGCAAG |
| | CTCTCCCACCGCGA (SEQ ID NO: 33) |
| mTagBFP2 | ATGGTCAGCAAAGGAGAGGAACTTATAAAGGAAAATATGCATATGA |
| | AATTGTACATGGAAGGTACAGTCGATAATCACCATTTTAAGTGTAC |
| | CTCTGAAGGAGAGGGGAAACCTTATGAAGGAACTCAAACTATGAGG |
| | ATTAAAGTCGTGGAAGGTGGGCCACTTCCATTCGCGTTTGATATTC |
| | TCGCAACAAGTTTTCTGTATGGTTCCAAAACCTTTATTAATCATAC |
| | GCAAGGAATCCCTGATTTCTTTAAACAATCATTTCCCGAAGGCTTT |
| | ACCTGGGAACGCGTGACGACGTATGAGGATGGTGGAGTCTTGACTG |
| | CCACTCAAGATACTTCACTGCAAGATGGGTGTCTGATTTATAATGT |
| | GAAAATTCGCGGCGTCAATTTTACCTCTAATGGGCCAGTCATGCAA |
| | AAGAAGACTTTGGGTTGGGAAGCATTTACAGAAACCTTGTATCCAG |
| | CGGATGGTGGACTTGAGGGGAGAAATGATATGGCACTCAAACTGGT |
| | TGGCGGAAGTCACCTCATAGCTAATGCTAAAACGACTTACCGCTCT |
| | AAGAAGCCGGCCAAGAATCTGAAAATGCCCGGTGTGTATTACGTTG |
| | ATTATCGGCTCGAGCGGATTAAAGAAGCGAATAATGAAACATATGT |
| | GGAACAACATGAAGTTGCCGTCGCACGGTATTGTGATCTGCCATCA |
| | AAGCTCGGCCATAAACTGAAC (SEQ ID NO: 34) |
| Complete sequence | ATGGACTGGATATGGAGAATATTGTTTCTGGTGGGCGCTGCTACCG |
| (including signal | GGGCTCATAGTCAAGTTCAACTTCAACAAAGTGGGCCAGAACTCAA |
| peptide) | GAAACCAGGGGAAACTGTGAAAATTTCATGTAAAGCTTCAGGATAT |
| | CCATTTACAAATTATGGGATGAATTGGGTCAAGCAAGCTCCCGGAC |
| | AAGGTCTCAAATGGATGGGGTGGATAAATACGTCCACAGGTGAATC |
| | CACATTTGCGGATGACTTTAAAGGACGGTTTGATTTTAGTCTCGAG |
| | ACTTCTGCTAACACTGCTTATCTCCAAATTAATAATCTGAAATCTG |
| | AAGATTCCGCAACATATTTCTGTGCACGGTGGGAAGTCTATCATGG |
| | TTATGTCCCTTATTGGGGACAAGGTACAACTGTAACTGTATCTTCC |
| | GGTGGGGGGGGAAGTGGAGGAGGCGGCAGTGGTGGTGGCGGGTCTG |
| | ATATACAACTCACGCAATCTCATAAATTCCTTTCTACGTCCGTTGG |
| | AGATAGAGTCAGCATTACGTGTAAGGCTTCCCAAGATGTCTATAAT |
| | GCAGTCGCATGGTACCAACAGAAACCAGGGCAAAGTCCTAAATTGC |
| | TTATATATTCTGCTTCTTCCAGGTATACTGGTGTCCCTTCCCGCTT |
| | TACGGGGTCCGGATCTGGGCCTGATTTTACGTTTACTATTTCATCC |
| | GTCCAAGCGGAAGATCTCGCTGTCTATTTCTGTCAACAGCATTTTC |
| | GTACCCCTTTTACATTTGGAAGTGGAACGAAACTCGAGATAAAAGC |
| | ACTTAGTAATTCCATTATGTATTTTAGCCATTTTGTCCCAGTCTTC |
| | CCTTCCAGAAAACCTACTACAACACCAGCGCCACGCCCACCAACGC |
| | CTGCACCTACGATAGCATCTCAACCACTCAGTCTCCGACCAGAAGC |
| | GTCTAGGCCAGCGGCCGGCGGCGCGGTCCATACAAGAGGGCTTGAT |
| | AAACCATTTTGGGTACTCGTGGTTGTAGGTGGTGTTTTGGCTTGCT |
| | ATTCCCTGCTTGTTACAGTCGCGTTTATTATTTTCTGGGTTCGTTC |
| | TAAAAGATCTCGCCTTTTGCATTCTGATTATATGAATATGACTCCC |
| | CGCAGACCTGGGCCGACTAGAAAACATTATCAACCGTACGCTCCCC |
| | CGCGGGATTTTGCTGCATATAGAAGCCGGGTCAAATTTTCACGTTC |
| | CGCTGATGCACCCGCATATCAACAAGGACAAAATCAACTTTATAAT |
| | GAACTCAATCTCGGGCGGCGCGAAGAGTATGATGTTCTCGATAAAA |
| | GGCGGGGCCGAGATCCCGAAATGGGTGGGAAACCGCGGAGAAAGAA |
| | TCCTCAAGAGGGGCTTTACAATGAGTTGCAAAAGGATAAAATGGCG |
| | GAAGCTTATTCTGAAATTGGGATGAAAGGAGAACGCCGCAGAGGGA |
| | AAGGTCATGATGGGCTCTATCAAGGGCTCAGCACGGCAACTAAAGA |
| | TACTTATGATGCTCTTCATATGCAAGCTCTCCCACCGCGAGCTTCC |
| | GCTACAAACTTTTCCCTTCTCAAACAGGCTGGAGATGTAGAGGAGA |
| | ATCCAGGGCCGATGGTCAGCAAAGGAGAGGAACTTATAAAGGAAAA |
| | TATGCATATGAAATTGTACATGGAAGGTACAGTCGATAATCACCAT |
| | TTTAAGTGTACCTCTGAAGGAGAGGGGAAACCTTATGAAGGAACTC |
| | AAACTATGAGGATTAAAGTCGTGGAAGGTGGGCCACTTCCATTCGC |
| | GTTTGATATTCTCGCAACAAGTTTTCTGTATGGTTCCAAAACCTTT |
| | ATTAATCATACGCAAGGAATCCCTGATTTCTTTAAACAATCATTTC |
| | CCGAAGGCTTTACCTGGGAACGCGTGACGACGTATGAGGATGGTGG |
| | AGTCTTGACTGCCACTCAAGATACTTCACTGCAAGATGGGTGTCTG |
| | ATTTATAATGTGAAAATTCGCGGCGTCAATTTTACCTCTAATGGGC |
| | CAGTCATGCAAAAGAAGACTTTGGGTTGGGAAGCATTTACAGAAAC |
| | CTTGTATCCAGCGGATGGTGGACTTGAGGGGAGAAATGATATGGCA |
| | CTCAAACTGGTTGGCGGAAGTCACCTCATAGCTAATGCTAAAACGA |
| | CTTACCGCTCTAAGAAGCCGGCCAAGAATCTGAAAATGCCCGGTGT |
| | GTATTACGTTGATTATCGGCTCGAGCGGATTAAAGAAGCGAATAAT |
| | GAAACATATGTGGAACAACATGAAGTTGCCGTCGCACGGTATTGTG |
| | ATCTGCCATCAAAGCTCGGCCATAAACTGAAC (SEQ ID NO: |
| | 35) |

SUPPLEMENTAL TABLE 2
Complete sequence for Glycocalyx-editing (GE)
enzyme variants and leucine zipper^{12,80}

| | |
|---|---|
| StcE Δ35 | GCTAGCGCTGATAATAATTCAGCCATTTATTTCAATACCTCCCAGC |
| (without signal | CTATAAATGATCTGCAGGGTTCGTTGGCCGCAGAGGTGAAATTTGC |
| peptide) | ACAAAGCCAGATTTTACCCGCCCATCCTAAAGAAGGGGATAGTCAA |
| | CCACATCTGACCAGCCTGCGGAAAAGTCTGCTGCTTGTCCGTCCGG |
| | TGAAAGCTGATGATAAAACACCTGTTCAGGTGGAAGCCCGCGATGA |
| | TAATAATAAAATTCTCGGTACGTTAACCCTTTATCCTCCTTCATCA |
| | CTACCGGATACAATCTACCATCTGGATGGTGTTCCGGAAGGTGGTA |
| | TCGATTTCACACCTCATAATGGAACGAAAAAGATCATTAATACGGT |
| | GGCTGAAGTAAACAAACTCAGTGATGCCAGCGGGAGTTCTATTCAT |
| | AGCCATCTAACAAATAATGCACTGGTGGAGATCCATACTGCAAATG |
| | GTCGTTGGGTAAGAGACATTTATCTGCCGCAGGGACCCGACCTTGA |
| | AGGTAAGATGGTTCGCTTTGTTTCGTCTGCAGGCTATAGTTCAACG |
| | GTTTTTTATGGTGATCGAAAAGTCACACTCTCGGTGGGTAACACTC |
| | TTCTGTTCAAATATGTAAATGGTCAGTGGTTCCGCTCCGGTGAACT |
| | GGAGAATAATCGAATCACTTATGCTCAGCATATTTGGAGTGCTGAA |
| | CTGCCTGCGCACTGGATCGTGCCTGGTTTAAACTTGGTGATTAAAC |
| | AGGGCAATCTGAGCGGTCGCCTAAATGATATCAAGATTGGAGCACC |
| | GGGTGAGCTGTTGTTGCATACAATTGATATCGGGATGTTGACCACT |
| | CCCCGGGATCGCTTTGATTTTGCCAAAGACAAAGAAGCACATAGGG |
| | AATATTTCCAGACCATTCCTGTAAGTCGTATGATTGTTAATAATTA |
| | TGCGCCTCTACACCTAAAGGAAGTTATGTTACCAACCGGAGAGTTA |
| | TTGACAGATATGGATCCAGGAAATGGTGGGTGGCATAGTGGTACAA |
| | TGCGTCAAAGAATAGGTAAAGAATTGGTTTCGCATGGCATTGATAA |
| | TGCTAACTATGGTTTAAATAGTACCGCAGGCTTAGGGGAGAATAGT |
| | CATCCATATGTAGTTGCGCAATTAGCGGCACATAATAGCCGCGGTA |
| | ATTATGCTAATGGCATCCAGGTTCATGGTGGCTCCGGAGGTGGGGG |
| | AATTGTTACTTTAGATTCCACATTGGGGAATGAGTTCAGTCATGAA |
| | GTTGGTCATAATTATGGTCTTGGTCATTATGTAGATGGTTTCAAGG |
| | GTTCTGTACATCGTAGTGCAGAAAATAACAACTCAACTTGGGGATG |
| | GGATGGTGATAAAAAACGGTTTATTCCTAACTTTTATCCGTCTCAA |
| | ACAAATGAAAAGAGTTGTCTGAATAATCAGTGTCAAGAACCGTTTG |
| | ATGGACACAAATTTGGTTTTGACGCCATGGCGGGAGGCAGCCCTTT |
| | CTCTGCTGCAAACCGTTTCACAATGTATACTCCGAATTCATCGGCT |
| | ATCATCCAGCGTTTTTTTGAAAATAAAGCTGTGTTCGATAGCCGTT |
| | CCTCCACCGGCTTCAGCAAGTGGAATGCAGATACGCAGGAAATGGA |
| | ACCGTATGAACACACCATTGACCGTGCGGAGCAGATTACGGCTTCA |
| | GTCAATGAGCTAAGTGAAAGCAAAATGGCTGAGCTGATGGCAGAGT |
| | ACGCTGTCGTCAAAGTGCATATGTGGAACGGTAACTGGACAAGAAA |
| | CATCTATATCCCTACAGCCTCCGCAGATAATAGAGGCAGTATCCTG |
| | ACCATCAACCATGAGGCCGGTTATAATAGTTATCTGTTTATAAATG |
| | GTGACGAAAAGGTCGTTTCCCAGGGGTATAAAAAGAGCTTTGTTTC |
| | CGATGGTCAGTTCTGGAAAGAACGTGATGTGGTTGATACTCGTGAA |
| | GCGCGTAAGCCAGAGCAGTTTGGTGTTCCTGTGACGACCCTGGTGG |
| | GGTATTACGATCCGGAAGGCACGCTGTCAAGCTACATCTATCCTGC |
| | GATGTATGGTGCCTATGGCTTCACTTATTCCGATGATAGTCAGAAT |
| | CTATCCGATAACGACTGCCAGCTGCAGGTGGATACGAAAGAAGGGC |
| | AGTTGCGATTCAGACTGGCTAATCACCGGGCTAACAACACTGTAAT |
| | GAATAAGTTCCATATTAACGTGCCAACAGAAAGTCAGCCCACACAG |
| | GCCACATTGGTTTGCAATAACAAGATACTGGATACCAAATCGCTCA |
| | CACCTGCGCCAGAAGGACTTACCTATACTGTAAATGGGCAGGCACT |
| | TCCAGCAAAAGAAAACGAGGGATGCATCGTGTCCGTGAATTCAGGT |
| | AAACGTTACTGTTTGCCGGTTGGTCAACGGTCAGGATATAGCCTTC |
| | CTGACTGGATTGTTGGGCAGGAAGTCTATGTCGACAGCGGGGCTAA |
| | AGCGAAAGTGCTGCTTTCTGACTGGGATAACCTGTCCTATAACAGG |
| | ATTGGTGAGTTTGTAGGTAATGTGAACCCAGCTGATATGAAAAAAG |
| | TTAAAGCCTGGAACGGACAGTATTTGGACTTCAGTAAACCTAGGTC |
| | AATGAGGGTTGTATATAAA (SEQ ID NO: 36) |
| StcE ΔX409 | GCTAGCGCTGATAATAATTCAGCCATTTATTTCAATACCTCCCAGC |
| | CTATAAATGATCTGCAGGGTTCGTTGGCCGCAGAGGTGAAATTTGC |
| | ACAAAGCCAGATTTTACCCGCCCATCCTAAAGAAGGGGATAGTCAA |
| | CCACATCTGACCAGCCTGCGGAAAAGTCTGCTGCTTGTCCGTCCGG |
| | TGAAAGCTGATGATAAAACACCTGTTCAGGTGGAAGCCCGCGATGA |
| | TAATAATAAAATTCTCGGTACGTTAACCCTTTATCCTCCTTCATCA |
| | CTACCGGATACAATCTACCATCTGGATGGTGTTCCGGAAGGTGGTA |
| | TCGATTTCACACCTCATAATGGAACGAAAAAGATCATTAATACGGT |
| | GGCTGAAGTAAACAAACTCAGTGATGCCAGCGGGAGTTCTATTCAT |
| | AGCCATCTAACAAATAATGCACTGGTGGAGATCCATACTGCAAATG |
| | GTCGTTGGGTAAGAGACATTTATCTGCCGCAGGGACCCGACCTTGA |
| | AGGTAAGATGGTTCGCTTTGTTTCGTCTGCAGGCTATAGTTCAACG |
| | GTTTTTTATGGTGATCGAAAAGTCACACTCTCGGTGGGTAACACTC |
| | TTCTGTTCAAATATGTAAATGGTCAGTGGTTCCGCTCCGGTGAACT |
| | GGAGAATAATCGAATCACTTATGCTCAGCATATTTGGAGTGCTGAA |
| | CTGCCTGCGCACTGGATCGTGCCTGGTTTAAACTTGGTGATTAAAC |
| | AGGGCAATCTGAGCGGTCGCCTAAATGATATCAAGATTGGAGCACC |
| | GGGTGAGCTGTTGTTGCATACAATTGATATCGGGATGTTGACCACT |
| | CCCCGGGATCGCTTTGATTTTGCCAAAGACAAAGAAGCACATAGGG |
| | AATATTTCCAGACCATTCCTGTAAGTCGTATGATTGTTAATAATTA |
| | TGCGCCTCTACACCTAAAGGAAGTTATGTTACCAACCGGAGAGTTA |
| | TTGACAGATATGGATCCAGGAAATGGTGGGTGGCATAGTGGTACAA |
| | TGCGTCAAAGAATAGGTAAAGAATTGGTTTCGCATGGCATTGATAA |
| | TGCTAACTATGGTTTAAATAGTACCGCAGGCTTAGGGGAGAATAGT |
| | CATCCATATGTAGTTGCGCAATTAGCGGCACATAATAGCCGCGGTA |
| | ATTATGCTAATGGCATCCAGGTTCATGGTGGCTCCGGAGGTGGGGG |
| | AATTGTTACTTTAGATTCCACATTGGGGAATGAGTTCAGTCATGAA |
| | GTTGGTCATAATTATGGTCTTGGTCATTATGTAGATGGTTTCAAGG |
| | GTTCTGTACATCGTAGTGCAGAAAATAACAACTCAACTTGGGGATG |
| | GGATGGTGATAAAAAACGGTTTATTCCTAACTTTTATCCGTCTCAA |
| | ACAAATGAAAAGAGTTGTCTGAATAATCAGTGTCAAGAACCGTTTG |
| | ATGGACACAAATTTGGTTTTGACGCCATGGCGGGAGGCAGCCCTTT |
| | CTCTGCTGCAAACCGTTTCACAATGTATACTCCGAATTCATCGGCT |
| | ATCATCCAGCGTTTTTTTGAAAATAAAGCTGTGTTCGATAGCCGTT |
| | CCTCCACCGGCTTCAGCAAGTGGAATGCAGATACGCAGGAAATGGA |
| | ACCGTATGAACACACCATTGACCGTGCGGAGCAGATTACGGCTTCA |
| | GTCAATGAGCTAAGTGAAAGCAAAATGGCTGAGCTGATGGCAGAGT |
| | ACGCTGTCGTCAAAGTGCATATGTGGAACGGTAACTGGACAAGAAA |
| | CATCTATATCCCTACAGCCTCCGCAGATAATAGAGGCAGTATCCTG |
| | ACCATCAACCATGAGGCCGGTTATAATAGTTATCTGTTTATAAATG |
| | GTGACGAAAAGGTCGTTTCCCAGGGGTATAAAAAGAGCTTTGTTTC |
| | CGATGGTCAGTTCTGGAAAGAACGTGATGTGGTTGATACTCGTGAA |
| | GCGCGTAAGCCAGAGCAGTTTGGTGTTCCTGTGACGACCCTGGTGG |
| | GGTATTACGATCCGGAAGGCACGCTGTCAAGCTACATCTATCCTGC |
| | GATGTATGGTGCCTATGGCTTCACTTATTCCGATGATAGTCAGAAT |
| | CTATCCGATAACGACTGCCAGCTGCAGGTGGATACGAAAGAAGGGC |
| | AGTTGCGATTCAGACTGGCTAATCACCGGGCTAACAACACTGTAAT |
| | GAATAAGTTCCATATTAACGTGCCAACAGAAAGTCAGCCCACACAG |
| | GCCACATTGGTTTGCAATAACAAGATACTGGATACCAAATCGCTCA |
| | CACCTGCGCCAGAAGGACTTACCTATACTGTAAAT (SEQ ID |
| | NO: 37) |
| AM0627 (21-504; | GCGAACACGCCGGAGCATATCGGGAATGATTTAAAACTGTTTAAAG |
| without signal | ATTCATCGTGTACCTCTTTGAAGCCGGACGTAAAAAATACTAGTGC |
| peptide) | CTTTCAGAGTGATGCGATGAAGGAATTGGCCACAAAGATCCTTGCC |
| | GGGCATTACAAACCTGATTATCTTTACGCGGAGTATCGCGCACTGC |
| | CGAGTCCTCGGCAGACAGGGAAAAATCTTCGCATCGGTGACGGGTT |
| | TAGTAAATATGACAACATGACCGGTGTGTACTTGGAGAAAGGCCGG |
| | CACGTCGTGTTGGTTGGGAAGACAGAGGGCCAGGAGATTTCCTTAC |
| | TGTTGCCGAACTTGATGCGGAAGCCGGCAGAAGGCGTCCAGCCTAC |
| | CAAGGACCCGAACGGCTGGGGCTTGCACAAAAAACAAATCCCACTC |
| | AAGGAGGGTATCAACATCATCGACGTAGAGACCCCGGCAAATGCAT |
| | ATATCTCATATTTCACTGAAGATGCCGGGAAAGCACCGAAGATCCC |
| | TGTACACTTCGTTACGGGCAAAGCTAACGGCTATTTTGACACCACC |
| | CGTGGGGACACGAACAAAGACTGGGTGCGTCTCTTGGATCAAGCTG |
| | TCAGCCCGATCATGGATGCACGGGGTAAATATATTCAAGTCGCATA |
| | TCCAGTGGAGTTCCTTAAAAAGTTCACAAAAGATCGGGGGACTGAG |
| | CTGATTAACGCATACGACAAGCTTATCGGGATCCAGTATCAGCTGA |
| | TGGGCTTAGATAAATATGGTAAAATTCCGGAAAATCGCGTATTGGC |
| | ACGCGTAAATTTTAACTACTATATGTTTCGTGATGGTGACGGGGTA |
| | GCCTATCTCGGCAACGATGGTACCATGCGTATGGTCACGGACCCAG |
| | AGAATGTATTGAAGGGTGACGCCTGCTGGGGCTTCTCGCACGAGGT |
| | GGGTCATGTTATGCAGATGCGGCCGATGACATGGGGTGGTATGACC |
| | GAGGTGTCCAACAATATCTTTAGCTTGCAAGCGGCAGCAAAGACTG |
| | GCAATGAGTCTCGTTTAAAACGGCAGGGTAGCTACGACAAAGCTCG |
| | CAAGGAAATTATTGAGGGTGAAATCGCGTATTTGCAATCGAAAGAC |
| | GTTTTTAACAAGTTGGTACCGTTATGGCAATTACATCTGTACTTTA |
| | CGAAGAACGGCCATCCAGATTTCTATCCAGATGTAATGGAATATCT |
| | GCGTAATAACGCTGGCAATTATGGTGGTAACGACACAGTCAAATAT |
| | CAGTTCGAGTTCGTTAAAGCGTGTTGCGATGTCACGAAGACCGATC |
| | TTACGGATTTTTTTGAAAAATGGGGCTTCTTTAAGCCTGGGAAATT |
| | TCACATCGGTGATTACGCCCAGTATGACTTTAACGTAACACCAGAG |
| | ATGGTTGAAGAGACCAAAAAATGGATTGCTGGGAAGGGGTACCCAA |
| | AACCTGAAACTGACATCACGGAGTTGTCTGAA (SEQ ID NO: |
| | 38) |
| BT4244 (35-857; | TTTTCAATTTCCGAGGAGTATTTAATTCAAAATTTAGACAAGTCCT |
| without signal | CCACATCCGTACAAATCCCTATTAATACTTCAATGGAGCTTGCGCA |
| peptide) | GTGGTCCGTGTCGTACGAAGCGAATTGGTTACAATGCTCTAAGCAA |
| | AAAACAGCCGCGGAAGGGACGTTTCTCCGCATTACGGTGAACGAAA |
| | ATACTGGGGAGACAAAGCGGACGGCTAATATCAAGGTCACCTCAAC |
| | AACAGCCACTTACACCATTACAGTGAATCAATATGCCAAAGGTGAA |
| | GTAATTGTAGAAGGTGATATTAAGGTGACTCCTACGGGGGGCAAAG |
| | CTTCGGAGCACCAAGAGGGTCAGGATATTGAGAACACTTATGATGG |
| | TAAATTTAGTACAGATGGTGCTGCCCCATTCCACACGCCGTGGGGG |
| | CAGTCCGCAAAGTTTCCAGTTACTCTGGAATACTACTTCAAGGGCG |
| | ACACTGAGATTGATTACTTGATTTACTATACGCGTTCAGGCAATGG |
| | CAATTTCGGTAAGGTGAAGGTGTATACCACAACAAATCCAGACCGG |
| | TCGGACTATACCCTTCAAGGGGAATACGACTTTAAGGAGCAAAACG |
| | CACCTTCAAAAGTCTCGTTTTCAGAAGGGATTAAGGCGACTGGCAT |
| | TAAGTTTGAGGTGTTATCTGGGTTGGGTGATTTCGTATCCTGTGAC |
| | GAGATGGAATTTTATAAAACGAACACTGACAAGACGTTGGACAAAC |
| | AATTGTTAACTGTCTTTACCGACATTACCTGTACTGAGATTAAGAA |
| | TAATGTGACAAACGAACAAATTCAGGCTTTACCAGATTATTTCGTG |
| | CGTATCGCCGAGGCGGTGCGGGATAATACTTATGACAAATGGGAGA |
| | AGGAGTTCCGTATCCGCAGTTACGAGCCTTACAGTAACATTGCTGA |
| | GTGGGCTGACAAACTTATGACTAAAAAATATTCAGATTTGGACAAT |
| | CCGACAGGCATCTCAGTGAAGGCTGGCGATGATATCATCGTTCTCG |
| | TGGGCGACACTTACGGGCAGAATATCTCTATGCAGTGCATTTGGGA |
| | GACGGGGACTGAGTACAAGCAGACAGCTAGCAGCGGGGACGTATAT |
| | ATGCTGAACCCGGGGGTAAACAAACTGACAATGAAGGGCGAGGGTC |
| | AGTTGTTCGTTATGTATAATACTGAATTAACTTCTAACACTGCTAA |
| | GCCAATCAAAATTCATATCCCATTGGGGTCCGGGACGGTAAACGGG |
| | TTTTTCGATTTAAAGGAGCATAAAACTGATGAGAAATACGCCGAAC |
| | TTTTGAAAAAATCGACACACAAGTATTTCTGTATCCGTGGGGAGAA |
| | GATCATGTTCTATTTCCACCGCAATAAGCTTTTAGAATACGTCCCA |
| | AACAACATTCTTTCTGCGATCCACCTGTGGGATAACATCGTAGGTT |
| | GGCAGCAAGAGCTTATGGGCATTGACGATGTGCGCCCATCGCAGGT |
| | CAACAACCATCTGTTCGCGATCTCGCCAGAAGGTAGTTACATGTGG |
| | GCCAGTGATTATCAGATTGGCTTCGTTTACACTTACTTAGGTAATA |
| | TTTTGCTCGAGGACAATGTTATGGCAGCAGAAGACAATGCTTGGGG |
| | TCCAGCACACGAGATCGGCCATGTTCATCAAGCTGCGATTAATTGG |
| | GCCTCCAGTACAGAATCATCAAACAATCTTTTCTCTAACTTCATCA |
| | TCTATAAGCTGGGCAAGTACAAGAGTCGGGGCAATGGTCTGGGTTC |
| | GGTGGCAACAGCGCGGTACGCTAATGGGCAGGCGTGGTACAACATG |
| | GGTGACGCCACTCACCAGAATGAGGACACGGAAACACATATGCGGA |
| | TGAATTGGCAATTATGGATCTATTACCATCGCTGTGAATACAAGAC |
| | TGACTTTTGGCAAACGTTGTTCAAGCTTATGCGGGAAGTCAACATG |
| | ACAGAGGGCGAAGACCCAGGCAAAAAGCAATTAGAATTTGCAAAGA |
| | TGGCGTCCAAGGCCGCCAACCAGAATCTTACAGATTTCTTCGAAAT |
| | GTGGGGCTTTTTTGAGCCGGTCAATACAACTATTGAGCAATACGGG |
| | ACGTATAAATACTACGTCTCTGATGCCATGATCCGGGAAGCTAAGG |
| | AATACATGGCACAATTTCCGGCGCCTAAGCACGCATTCCAATATAT |
| | CGAGGACCGCAAGAAGTCCGAGTTCCCTTCGAACGACTATCGCTAC |
| | TCGGCCGTTGGTGATGTCGGCTATTATACCCAGTTTAAAGAAAATC |
| | AAAAGATTACCAAGGCGATTACGGCTGAACTGGCAGGGCGCAAGGT |
| | TTCCATCCAGAATGGCGACGAAGCGGTTGCCTTTGAGTTACGGGAA |
| | AATGACGAGAACGGCAAGTTATTGTATTTTAGCACTTTCACCACAT |
| | TTGAAATCCCTAGTTCCATTCTCATGGTGAATGCGAAGTTATATGC |
| | CGTACAGGCGGACGGCAAACGTATCCTCCTG (SEQ ID NO: |
| | 39) |
| ST Sialidase | ACCGTAGAGAAGTCAGTGGTGTTCAAAGCCGAAGGCGAGCATTTTA |
| (S. typhimurium) | CCGATCAGAAAGGAAATACCATTGTGGGTTCTGGGTCGGGTGGGAC |
| | TACAAAATATTTCCGTATCCCGGCAATGTGTACCACATCAAAGGGT |
| | ACGATCGTTGTTTTCGCTGACGCCCGCCATAATACTGCGTCTGATC |
| | AAAGTTTCATCGATACAGCCGCCGCACGTAGCACGGACGGCGGGAA |
| | AACTTGGAATAAAAAAATCGCTATCTACAATGACCGCGTCAATAGC |
| | AAGCTGTCTCGTGTTATGGATCCTACTTGCATTGTCGCTAATATTC |
| | AGGGTCGCGAGACCATCTTAGTTATGGTAGGAAAGTGGAACAACAA |
| | TGACAAAACTTGGGGTGCTTATCGCGACAAAGCCCCTGATACTGAT |
| | TGGGATCTGGTTTTATACAAGTCAACCGACGACGGAGTAACTTTCA |
| | GCAAGGTTGAAACTAATATTCACGATATCGTAACCAAAAATGGAAC |
| | AATTTCAGCAATGTTAGGTGGCGTAGGGAGCGGATTACAACTTAAC |
| | GACGGAAAACTGGTCTTCCCGGTACAAATGGTCCGCACAAAAAATA |
| | TCACGACTGTCTTGAACACCTCCTTTATCTACTCAACCGACGGGAT |
| | CACCTGGAGTTTACCTTCTGGATACTGCGAGGGTTTCGGCTCCGAA |
| | AACAACATCATTGAATTCAATGCGTCTTTGGTCAACAACATCCGCA |
| | ATTCAGGGTTGCGCCGTTCGTTTGAGACAAAGGACTTCGGGAAGAC |
| | CTGGACGGAGTTTCCGCCGATGGACAAAAAAGTGGACAATCGTAAT |
| | CATGGCGTCCAAGGCAGCACTATTACGATTCCTTCAGGAAACAAAC |
| | TGGTGGCTGCCCACAGTTCTGCCCAGAATAAAAACAATGATTACAC |
| | TCGCTCTGATATTTCACTGTACGCTCATAACCTTTATTCAGGAGAG |
| | GTTAAATTGATTGACGCATTTTACCCGAAGGTCGGCAACGCATCTG |
| | GGGCCGGGTATAGCTGTTTGAGCTACCGCAAAAACGTTGACAAGGA |
| | GACCTTGTATGTAGTGTACGAAGCAAATGGCTCTATTGAGTTCCAA |
| | GACCTGTCGCGCCATCTGCCCGTCATTAAGTCGTACAAC (SEQ |
| | ID NO: 40) |
| EE leucine | ATGGACCCTGATCTTGAGATTGAAGCTGCCTTCCTTGAACGGGAGA |
| zipper | ACACGGCCTTGGAAACGAGAGTGGCGGAGTTAAGACAAAGAGTCCA |
| | GCGCCTTCGTAACCGCGTAAGCCAGTATCGGACCCGGTACGGACCC |
| | CTTGGCGGGGGAAAG (SEQ ID NO: 41) |
| RR leucine | ATGGACCCTGATCTCGAAATTCGCGCGGCCTTCCTCAGGCAGCGAA |
| zipper | ATACCGCTTTGAGAACGGAAGTCGCTGAGCTCGAACAAGAGGTCCA |
| | GAGACTGGAGAACGAAGTGAGCCAATATGAAACACGATATGGCCCC |
| | CTCGGCGGCGGAAAG (SEQ ID NO: 42) |
| StcE ΔX409- | GCTAGCGCTGATAATAATTCAGCCATTTATTTCAATACCTCCCAGC |
| 35 aa Linker- | CTATAAATGATCTGCAGGGTTCGTTGGCCGCAGAGGTGAAATTTGC |
| EE Zip | ACAAAGCCAGATTTTACCCGCCCATCCTAAAGAAGGGGATAGTCAA |
| | CCACATCTGACCAGCCTGCGGAAAAGTCTGCTGCTTGTCCGTCCGG |
| | TGAAAGCTGATGATAAAACACCTGTTCAGGTGGAAGCCCGCGATGA |
| | TAATAATAAAATTCTCGGTACGTTAACCCTTTATCCTCCTTCATCA |
| | CTACCGGATACAATCTACCATCTGGATGGTGTTCCGGAAGGTGGTA |
| | TCGATTTCACACCTCATAATGGAACGAAAAAGATCATTAATACGGT |
| | GGCTGAAGTAAACAAACTCAGTGATGCCAGCGGGAGTTCTATTCAT |
| | AGCCATCTAACAAATAATGCACTGGTGGAGATCCATACTGCAAATG |
| | GTCGTTGGGTAAGAGACATTTATCTGCCGCAGGGACCCGACCTTGA |
| | AGGTAAGATGGTTCGCTTTGTTTCGTCTGCAGGCTATAGTTCAACG |
| | GTTTTTTATGGTGATCGAAAAGTCACACTCTCGGTGGGTAACACTC |
| | TTCTGTTCAAATATGTAAATGGTCAGTGGTTCCGCTCCGGTGAACT |
| | GGAGAATAATCGAATCACTTATGCTCAGCATATTTGGAGTGCTGAA |
| | CTGCCTGCGCACTGGATCGTGCCTGGTTTAAACTTGGTGATTAAAC |
| | AGGGCAATCTGAGCGGTCGCCTAAATGATATCAAGATTGGAGCACC |
| | GGGTGAGCTGTTGTTGCATACAATTGATATCGGGATGTTGACCACT |
| | CCCCGGGATCGCTTTGATTTTGCCAAAGACAAAGAAGCACATAGGG |
| | AATATTTCCAGACCATTCCTGTAAGTCGTATGATTGTTAATAATTA |
| | TGCGCCTCTACACCTAAAGGAAGTTATGTTACCAACCGGAGAGTTA |
| | TTGACAGATATGGATCCAGGAAATGGTGGGTGGCATAGTGGTACAA |
| | TGCGTCAAAGAATAGGTAAAGAATTGGTTTCGCATGGCATTGATAA |
| | TGCTAACTATGGTTTAAATAGTACCGCAGGCTTAGGGGAGAATAGT |
| | CATCCATATGTAGTTGCGCAATTAGCGGCACATAATAGCCGCGGTA |
| | ATTATGCTAATGGCATCCAGGTTCATGGTGGCTCCGGAGGTGGGGG |
| | AATTGTTACTTTAGATTCCACATTGGGGAATGAGTTCAGTCATGAA |
| | GTTGGTCATAATTATGGTCTTGGTCATTATGTAGATGGTTTCAAGG |
| | GTTCTGTACATCGTAGTGCAGAAAATAACAACTCAACTTGGGGATG |
| | GGATGGTGATAAAAAACGGTTTATTCCTAACTTTTATCCGTCTCAA |
| | ACAAATGAAAAGAGTTGTCTGAATAATCAGTGTCAAGAACCGTTTG |
| | ATGGACACAAATTTGGTTTTGACGCCATGGCGGGAGGCAGCCCTTT |
| | CTCTGCTGCAAACCGTTTCACAATGTATACTCCGAATTCATCGGCT |
| | ATCATCCAGCGTTTTTTTGAAAATAAAGCTGTGTTCGATAGCCGTT |
| | CCTCCACCGGCTTCAGCAAGTGGAATGCAGATACGCAGGAAATGGA |
| | ACCGTATGAACACACCATTGACCGTGCGGAGCAGATTACGGCTTCA |
| | GTCAATGAGCTAAGTGAAAGCAAAATGGCTGAGCTGATGGCAGAGT |
| | ACGCTGTCGTCAAAGTGCATATGTGGAACGGTAACTGGACAAGAAA |
| | CATCTATATCCCTACAGCCTCCGCAGATAATAGAGGCAGTATCCTG |
| | ACCATCAACCATGAGGCCGGTTATAATAGTTATCTGTTTATAAATG |
| | GTGACGAAAAGGTCGTTTCCCAGGGGTATAAAAAGAGCTTTGTTTC |
| | CGATGGTCAGTTCTGGAAAGAACGTGATGTGGTTGATACTCGTGAA |
| | GCGCGTAAGCCAGAGCAGTTTGGTGTTCCTGTGACGACCCTGGTGG |
| | GGTATTACGATCCGGAAGGCACGCTGTCAAGCTACATCTATCCTGC |
| | GATGTATGGTGCCTATGGCTTCACTTATTCCGATGATAGTCAGAAT |
| | CTATCCGATAACGACTGCCAGCTGCAGGTGGATACGAAAGAAGGGC |
| | AGTTGCGATTCAGACTGGCTAATCACCGGGCTAACAACACTGTAAT |
| | GAATAAGTTCCATATTAACGTGCCAACAGAAAGTCAGCCCACACAG |
| | GCCACATTGGTTTGCAATAACAAGATACTGGATACCAAATCGCTCA |
| | CACCTGCGCCAGAAGGACTTACCTATACTGTAAATGGTGGAGGCGG |
| | TTCTGGCGGTGGCGGCTCAGGTGGAGGTGGCTCTGGCGGAGGAGGT |
| | AGCGGAGGTGGCGGTAGCGGCGGAGGAGGTTCTGGTGGAGGAGGTA |
| | GCATGGACCCTGATCTTGAGATTGAAGCTGCCTTCCTTGAACGGGA |
| | GAACACGGCCTTGGAAACGAGAGTGGCGGAGTTAAGACAAAGAGTC |
| | CAGCGCCTTCGTAACCGCGTAAGCCAGTATCGGACCCGGTACGGAC |
| | CCCTTGGCGGGGGAAAG (SEQ ID NO: 43) |
| AM0627- | GCGAACACGCCGGAGCATATCGGGAATGATTTAAAACTGTTTAAAG |
| 35 aa Linker - | ATTCATCGTGTACCTCTTTGAAGCCGGACGTAAAAAATACTAGTGC |
| EE Zip | CTTTCAGAGTGATGCGATGAAGGAATTGGCCACAAAGATCCTTGCC |
| | GGGCATTACAAACCTGATTATCTTTACGCGGAGTATCGCGCACTGC |
| | CGAGTCCTCGGCAGACAGGGAAAAATCTTCGCATCGGTGACGGGTT |
| | TAGTAAATATGACAACATGACCGGTGTGTACTTGGAGAAAGGCCGG |
| | CACGTCGTGTTGGTTGGGAAGACAGAGGGCCAGGAGATTTCCTTAC |
| | TGTTGCCGAACTTGATGCGGAAGCCGGCAGAAGGCGTCCAGCCTAC |
| | CAAGGACCCGAACGGCTGGGGCTTGCACAAAAAACAAATCCCACTC |
| | AAGGAGGGTATCAACATCATCGACGTAGAGACCCCGGCAAATGCAT |
| | ATATCTCATATTTCACTGAAGATGCCGGGAAAGCACCGAAGATCCC |
| | TGTACACTTCGTTACGGGCAAAGCTAACGGCTATTTTGACACCACC |
| | CGTGGGGACACGAACAAAGACTGGGTGCGTCTCTTGGATCAAGCTG |
| | TCAGCCCGATCATGGATGCACGGGGTAAATATATTCAAGTCGCATA |
| | TCCAGTGGAGTTCCTTAAAAAGTTCACAAAAGATCGGGGGACTGAG |
| | CTGATTAACGCATACGACAAGCTTATCGGGATCCAGTATCAGCTGA |
| | TGGGCTTAGATAAATATGGTAAAATTCCGGAAAATCGCGTATTGGC |
| | ACGCGTAAATTTTAACTACTATATGTTTCGTGATGGTGACGGGGTA |
| | GCCTATCTCGGCAACGATGGTACCATGCGTATGGTCACGGACCCAG |
| | AGAATGTATTGAAGGGTGACGCCTGCTGGGGCTTCTCGCACGAGGT |
| | GGGTCATGTTATGCAGATGCGGCCGATGACATGGGGTGGTATGACC |
| | GAGGTGTCCAACAATATCTTTAGCTTGCAAGCGGCAGCAAAGACTG |
| | GCAATGAGTCTCGTTTAAAACGGCAGGGTAGCTACGACAAAGCTCG |
| | CAAGGAAATTATTGAGGGTGAAATCGCGTATTTGCAATCGAAAGAC |
| | GTTTTTAACAAGTTGGTACCGTTATGGCAATTACATCTGTACTTTA |
| | CGAAGAACGGCCATCCAGATTTCTATCCAGATGTAATGGAATATCT |
| | GCGTAATAACGCTGGCAATTATGGTGGTAACGACACAGTCAAATAT |
| | CAGTTCGAGTTCGTTAAAGCGTGTTGCGATGTCACGAAGACCGATC |
| | TTACGGATTTTTTTGAAAAATGGGGCTTCTTTAAGCCTGGGAAATT |
| | TCACATCGGTGATTACGCCCAGTATGACTTTAACGTAACACCAGAG |
| | ATGGTTGAAGAGACCAAAAAATGGATTGCTGGGAAGGGGTACCCAA |
| | AACCTGAAACTGACATCACGGAGTTGTCTGAAGGTGGAGGCGGTTC |
| | TGGCGGTGGCGGCTCAGGTGGAGGTGGCTCTGGCGGAGGAGGTAGC |
| | GGAGGTGGCGGTAGCGGCGGAGGAGGTTCTGGTGGAGGAGGTAGCA |
| | TGGACCCTGATCTTGAGATTGAAGCTGCCTTCCTTGAACGGGAGAA |
| | CACGGCCTTGGAAACGAGAGTGGCGGAGTTAAGACAAAGAGTCCAG |
| | CGCCTTCGTAACCGCGTAAGCCAGTATCGGACCCGGTACGGACCCC |
| | TTGGCGGGGGAAAG (SEQ ID NO: 44) |
| BT4244- | TTTTCAATTTCCGAGGAGTATTTAATTCAAAATTTAGACAAGTCCT |
| 35 aa Linker - | CCACATCCGTACAAATCCCTATTAATACTTCAATGGAGCTTGCGCA |
| EE Zip | GTGGTCCGTGTCGTACGAAGCGAATTGGTTACAATGCTCTAAGCAA |
| | AAAACAGCCGCGGAAGGGACGTTTCTCCGCATTACGGTGAACGAAA |
| | ATACTGGGGAGACAAAGCGGACGGCTAATATCAAGGTCACCTCAAC |
| | AACAGCCACTTACACCATTACAGTGAATCAATATGCCAAAGGTGAA |
| | GTAATTGTAGAAGGTGATATTAAGGTGACTCCTACGGGGGGCAAAG |
| | CTTCGGAGCACCAAGAGGGTCAGGATATTGAGAACACTTATGATGG |
| | TAAATTTAGTACAGATGGTGCTGCCCCATTCCACACGCCGTGGGGG |
| | CAGTCCGCAAAGTTTCCAGTTACTCTGGAATACTACTTCAAGGGCG |
| | ACACTGAGATTGATTACTTGATTTACTATACGCGTTCAGGCAATGG |
| | CAATTTCGGTAAGGTGAAGGTGTATACCACAACAAATCCAGACCGG |
| | TCGGACTATACCCTTCAAGGGGAATACGACTTTAAGGAGCAAAACG |
| | CACCTTCAAAAGTCTCGTTTTCAGAAGGGATTAAGGCGACTGGCAT |
| | TAAGTTTGAGGTGTTATCTGGGTTGGGTGATTTCGTATCCTGTGAC |
| | GAGATGGAATTTTATAAAACGAACACTGACAAGACGTTGGACAAAC |
| | AATTGTTAACTGTCTTTACCGACATTACCTGTACTGAGATTAAGAA |
| | TAATGTGACAAACGAACAAATTCAGGCTTTACCAGATTATTTCGTG |
| | CGTATCGCCGAGGCGGTGCGGGATAATACTTATGACAAATGGGAGA |
| | AGGAGTTCCGTATCCGCAGTTACGAGCCTTACAGTAACATTGCTGA |
| | GTGGGCTGACAAACTTATGACTAAAAAATATTCAGATTTGGACAAT |
| | CCGACAGGCATCTCAGTGAAGGCTGGCGATGATATCATCGTTCTCG |
| | TGGGCGACACTTACGGGCAGAATATCTCTATGCAGTGCATTTGGGA |
| | GACGGGGACTGAGTACAAGCAGACAGCTAGCAGCGGGGACGTATAT |
| | ATGCTGAACCCGGGGGTAAACAAACTGACAATGAAGGGCGAGGGTC |
| | AGTTGTTCGTTATGTATAATACTGAATTAACTTCTAACACTGCTAA |
| | GCCAATCAAAATTCATATCCCATTGGGGTCCGGGACGGTAAACGGG |
| | TTTTTCGATTTAAAGGAGCATAAAACTGATGAGAAATACGCCGAAC |
| | TTTTGAAAAAATCGACACACAAGTATTTCTGTATCCGTGGGGAGAA |
| | GATCATGTTCTATTTCCACCGCAATAAGCTTTTAGAATACGTCCCA |
| | AACAACATTCTTTCTGCGATCCACCTGTGGGATAACATCGTAGGTT |
| | GGCAGCAAGAGCTTATGGGCATTGACGATGTGCGCCCATCGCAGGT |
| | CAACAACCATCTGTTCGCGATCTCGCCAGAAGGTAGTTACATGTGG |
| | GCCAGTGATTATCAGATTGGCTTCGTTTACACTTACTTAGGTAATA |
| | TTTTGCTCGAGGACAATGTTATGGCAGCAGAAGACAATGCTTGGGG |
| | TCCAGCACACGAGATCGGCCATGTTCATCAAGCTGCGATTAATTGG |
| | GCCTCCAGTACAGAATCATCAAACAATCTTTTCTCTAACTTCATCA |
| | TCTATAAGCTGGGCAAGTACAAGAGTCGGGGCAATGGTCTGGGTTC |
| | GGTGGCAACAGCGCGGTACGCTAATGGGCAGGCGTGGTACAACATG |
| | GGTGACGCCACTCACCAGAATGAGGACACGGAAACACATATGCGGA |
| | TGAATTGGCAATTATGGATCTATTACCATCGCTGTGAATACAAGAC |
| | TGACTTTTGGCAAACGTTGTTCAAGCTTATGCGGGAAGTCAACATG |
| | ACAGAGGGCGAAGACCCAGGCAAAAAGCAATTAGAATTTGCAAAGA |
| | TGGCGTCCAAGGCCGCCAACCAGAATCTTACAGATTTCTTCGAAAT |
| | GTGGGGCTTTTTTGAGCCGGTCAATACAACTATTGAGCAATACGGG |
| | ACGTATAAATACTACGTCTCTGATGCCATGATCCGGGAAGCTAAGG |
| | AATACATGGCACAATTTCCGGCGCCTAAGCACGCATTCCAATATAT |
| | CGAGGACCGCAAGAAGTCCGAGTTCCCTTCGAACGACTATCGCTAC |
| | TCGGCCGTTGGTGATGTCGGCTATTATACCCAGTTTAAAGAAAATC |
| | AAAAGATTACCAAGGCGATTACGGCTGAACTGGCAGGGCGCAAGGT |
| | TTCCATCCAGAATGGCGACGAAGCGGTTGCCTTTGAGTTACGGGAA |
| | AATGACGAGAACGGCAAGTTATTGTATTTTAGCACTTTCACCACAT |
| | TTGAAATCCCTAGTTCCATTCTCATGGTGAATGCGAAGTTATATGC |
| | CGTACAGGCGGACGGCAAACGTATCCTCCTGGGTGGAGGCGGTTCT |
| | GGCGGTGGCGGCTCAGGTGGAGGTGGCTCTGGCGGAGGAGGTAGCG |
| | GAGGTGGCGGTAGCGGCGGAGGAGGTTCTGGTGGAGGAGGTAGCAT |
| | GGACCCTGATCTTGAGATTGAAGCTGCCTTCCTTGAACGGGAGAAC |
| | ACGGCCTTGGAAACGAGAGTGGCGGAGTTAAGACAAAGAGTCCAGC |
| | GCCTTCGTAACCGCGTAAGCCAGTATCGGACCCGGTACGGACCCCT |
| | TGGCGGGGGAAAG (SEQ ID NO: 45) |
| ST Sialidase- | ACCGTAGAGAAGTCAGTGGTGTTCAAAGCCGAAGGCGAGCATTTTA |
| 35 aa Linker- | CCGATCAGAAAGGAAATACCATTGTGGGTTCTGGGTCGGGTGGGAC |
| EE Zip | TACAAAATATTTCCGTATCCCGGCAATGTGTACCACATCAAAGGGT |
| | ACGATCGTTGTTTTCGCTGACGCCCGCCATAATACTGCGTCTGATC |
| | AAAGTTTCATCGATACAGCCGCCGCACGTAGCACGGACGGCGGGAA |
| | AACTTGGAATAAAAAAATCGCTATCTACAATGACCGCGTCAATAGC |
| | AAGCTGTCTCGTGTTATGGATCCTACTTGCATTGTCGCTAATATTC |
| | AGGGTCGCGAGACCATCTTAGTTATGGTAGGAAAGTGGAACAACAA |
| | TGACAAAACTTGGGGTGCTTATCGCGACAAAGCCCCTGATACTGAT |
| | TGGGATCTGGTTTTATACAAGTCAACCGACGACGGAGTAACTTTCA |
| | GCAAGGTTGAAACTAATATTCACGATATCGTAACCAAAAATGGAAC |
| | AATTTCAGCAATGTTAGGTGGCGTAGGGAGCGGATTACAACTTAAC |
| | GACGGAAAACTGGTCTTCCCGGTACAAATGGTCCGCACAAAAAATA |
| | TCACGACTGTCTTGAACACCTCCTTTATCTACTCAACCGACGGGAT |
| | CACCTGGAGTTTACCTTCTGGATACTGCGAGGGTTTCGGCTCCGAA |
| | AACAACATCATTGAATTCAATGCGTCTTTGGTCAACAACATCCGCA |
| | ATTCAGGGTTGCGCCGTTCGTTTGAGACAAAGGACTTCGGGAAGAC |
| | CTGGACGGAGTTTCCGCCGATGGACAAAAAAGTGGACAATCGTAAT |
| | CATGGCGTCCAAGGCAGCACTATTACGATTCCTTCAGGAAACAAAC |
| | TGGTGGCTGCCCACAGTTCTGCCCAGAATAAAAACAATGATTACAC |
| | TCGCTCTGATATTTCACTGTACGCTCATAACCTTTATTCAGGAGAG |
| | GTTAAATTGATTGACGCATTTTACCCGAAGGTCGGCAACGCATCTG |
| | GGGCCGGGTATAGCTGTTTGAGCTACCGCAAAAACGTTGACAAGGA |
| | GACCTTGTATGTAGTGTACGAAGCAAATGGCTCTATTGAGTTCCAA |
| | GACCTGTCGCGCCATCTGCCCGTCATTAAGTCGTACAACGGTGGAG |
| | GCGGTTCTGGCGGTGGCGGCTCAGGTGGAGGTGGCTCTGGCGGAGG |
| | AGGTAGCGGAGGTGGCGGTAGCGGCGGAGGAGGTTCTGGTGGAGGA |
| | GGTAGCATGGACCCTGATCTTGAGATTGAAGCTGCCTTCCTTGAAC |
| | GGGAGAACACGGCCTTGGAAACGAGAGTGGCGGAGTTAAGACAAAG |
| | AGTCCAGCGCCTTCGTAACCGCGTAAGCCAGTATCGGACCCGGTAC |
| | GGACCCCTTGGCGGGGGAAAG (SEQ ID NO: 46) |
| Genetic | ATGGAGACAGACACACTCCTGCTATGGGTACTGCTGCTCTGGGTTC |
| expression | CAGGTTCCACTGGTGACTCAAGATTGGAGGAGGAATTGCGGAGACG |
| of Sialidase | ATTGACCGAGCCAACCGTAGAGAAGTCAGTGGTGTTCAAAGCCGAA |
| IgK leader- | GGCGAGCATTTTACCGATCAGAAAGGAAATACCATTGTGGGTTCTG |
| ALFA | GGTCGGGTGGGACTACAAAATATTTCCGTATCCCGGCAATGTGTAC |
| Tag-ST | CACATCAAAGGGTACGATCGTTGTTTTCGCTGACGCCCGCCATAAT |
| Sialidase- | ACTGCGTCTGATCAAAGTTTCATCGATACAGCCGCCGCACGTAGCA |
| PDGFRβ | CGGACGGCGGGAAAACTTGGAATAAAAAAATCGCTATCTACAATGA |
| TM - | CCGCGTCAATAGCAAGCTGTCTCGTGTTATGGATCCTACTTGCATT |
| sfGFP | GTCGCTAATATTCAGGGTCGCGAGACCATCTTAGTTATGGTAGGAA |
| | AGTGGAACAACAATGACAAAACTTGGGGTGCTTATCGCGACAAAGC |
| | CCCTGATACTGATTGGGATCTGGTTTTATACAAGTCAACCGACGAC |
| | GGAGTAACTTTCAGCAAGGTTGAAACTAATATTCACGATATCGTAA |
| | CCAAAAATGGAACAATTTCAGCAATGTTAGGTGGCGTAGGGAGCGG |
| | ATTACAACTTAACGACGGAAAACTGGTCTTCCCGGTACAAATGGTC |
| | CGCACAAAAAATATCACGACTGTCTTGAACACCTCCTTTATCTACT |
| | CAACCGACGGGATCACCTGGAGTTTACCTTCTGGATACTGCGAGGG |
| | TTTCGGCTCCGAAAACAACATCATTGAATTCAATGCGTCTTTGGTC |
| | AACAACATCCGCAATTCAGGGTTGCGCCGTTCGTTTGAGACAAAGG |
| | ACTTCGGGAAGACCTGGACGGAGTTTCCGCCGATGGACAAAAAAGT |
| | GGACAATCGTAATCATGGCGTCCAAGGCAGCACTATTACGATTCCT |
| | TCAGGAAACAAACTGGTGGCTGCCCACAGTTCTGCCCAGAATAAAA |
| | ACAATGATTACACTCGCTCTGATATTTCACTGTACGCTCATAACCT |
| | TTATTCAGGAGAGGTTAAATTGATTGACGCATTTTACCCGAAGGTC |
| | GGCAACGCATCTGGGGCCGGGTATAGCTGTTTGAGCTACCGCAAAA |
| | ACGTTGACAAGGAGACCTTGTATGTAGTGTACGAAGCAAATGGCTC |
| | TATTGAGTTCCAAGACCTGTCGCGCCATCTGCCCGTCATTAAGTCG |
| | TACAACTTGGAAGAACAAAAACTCATCTCAGAAGAGGATCTGAATG |
| | CTGTGGGCCAGGACACGCAGGAGGTCATCGTGGTGCCACACTCCTT |
| | GCCCTTTAAGGTGGTGGTGATCTCAGCCATCCTGGCCCTGGTGGTG |
| | CTCACCATCATCTCCCTTATCATCCTCATCATGCTTTGGCAGAAGA |
| | AGCCACGTACTAGTGGTTCTGGTTCTCCTGCAGGAATGAGCAAAGG |
| | AGAAGAACTTTTCACTGGAGTTGTCCCAATTCTTGTTGAATTAGAT |
| | GGTGATGTTAATGGGCACAAATTTTCTGTCCGTGGAGAGGGTGAAG |
| | GTGATGCTACAAACGGAAAACTCACCCTTAAATTTATTTGCACTAC |
| | TGGAAAACTACCTGTTCCGTGGCCAACACTTGTCACTACTCTGACC |
| | TATGGTGTTCAATGCTTTTCCCGTTATCCGGATCACATGAAACGGC |
| | ATGACTTTTTCAAGAGTGCCATGCCCGAAGGTTATGTACAGGAACG |
| | CACTATATCTTTCAAAGATGACGGGACCTACAAGACGCGTGCTGAA |
| | GTCAAGTTTGAAGGTGATACCCTTGTTAATCGTATCGAGTTAAAGG |
| | GTATTGATTTTAAAGAAGATGGAAACATTCTTGGACACAAACTCGA |
| | GTACAACTTTAACTCACACAATGTATACATCACGGCAGACAAACAA |
| | AAGAATGGAATCAAAGCTAACTTCAAAATTCGCCACAACGTTGAAG |
| | ATGGTTCCGTTCAACTAGCAGACCATTATCAACAAAATACTCCAAT |
| | TGGCGATGGCCCTGTCCTTTTACCAGACAACCATTACCTGTCGACA |
| | CAATCTGTCCTTTCGAAAGATCCCAACGAAAAGCGTGACCACATGG |
| | TCCTTCTTGAGTTTGTAACTGCTGCTGGGATTACACATGGCATGGA |
| | TGAGCTCTACAAA (SEQ ID NO: 47) |
| IgK leader- | ATGGAGACAGACACACTCCTGCTATGGGTACTGCTGCTCTGGGTTC |
| ALFA | CAGGTTCCACTGGTGACTCAAGATTGGAGGAGGAATTGCGGAGACG |
| Tag-RR | ATTGACCGAGCCAATGGACCCTGATCTCGAAATTCGCGCGGCCTTC |
| Zip-Myc | CTCAGGCAGCGAAATACCGCTTTGAGAACGGAAGTCGCTGAGCTCG |
| Tag- | AACAAGAGGTCCAGAGACTGGAGAACGAAGTGAGCCAATATGAAAC |
| PDGFRβ | ACGATATGGCCCCCTCGGCGGCGGAAAGTTGGAAGAACAAAAACTC |
| TM - | ATCTCAGAAGAGGATCTGAATGCTGTGGGCCAGGACACGCAGGAGG |
| sfGFP | TCATCGTGGTGCCACACTCCTTGCCCTTTAAGGTGGTGGTGATCTC |
| | AGCCATCCTGGCCCTGGTGGTGCTCACCATCATCTCCCTTATCATC |
| | CTCATCATGCTTTGGCAGAAGAAGCCACGTACTAGTGGTTCTGGTT |
| | CTATGAGCAAAGGAGAAGAACTTTTCACTGGAGTTGTCCCAATTCT |
| | TGTTGAATTAGATGGTGATGTTAATGGGCACAAATTTTCTGTCCGT |
| | GGAGAGGGTGAAGGTGATGCTACAAACGGAAAACTCACCCTTAAAT |
| | TTATTTGCACTACTGGAAAACTACCTGTTCCGTGGCCAACACTTGT |
| | CACTACTCTGACCTATGGTGTTCAATGCTTTTCCCGTTATCCGGAT |
| | CACATGAAACGGCATGACTTTTTCAAGAGTGCCATGCCCGAAGGTT |
| | ATGTACAGGAACGCACTATATCTTTCAAAGATGACGGGACCTACAA |
| | GACGCGTGCTGAAGTCAAGTTTGAAGGTGATACCCTTGTTAATCGT |
| | ATCGAGTTAAAGGGTATTGATTTTAAAGAAGATGGAAACATTCTTG |
| | GACACAAACTCGAGTACAACTTTAACTCACACAATGTATACATCAC |
| | GGCAGACAAACAAAAGAATGGAATCAAAGCTAACTTCAAAATTCGC |
| | CACAACGTTGAAGATGGTTCCGTTCAACTAGCAGACCATTATCAAC |
| | AAAATACTCCAATTGGCGATGGCCCTGTCCTTTTACCAGACAACCA |
| | TTACCTGTCGACACAATCTGTCCTTTCGAAAGATCCCAACGAAAAG |
| | CGTGACCACATGGTCCTTCTTGAGTTTGTAACTGCTGCTGGGATTA |
| | CACATGGCATGGATGAGCTCTACAAA (SEQ ID NO: 48) |

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A modified eukaryotic cell comprising a plasma membrane-coupled glycocalyx-editing (GE) enzyme.

2. The modified eukaryotic cell of claim 1, wherein the GE enzyme is a mucinase or a sialidase.

3. The modified eukaryotic cell of claim 2, wherein the GE enzyme is coupled to the plasma membrane by a transmembrane domain that is a component of the GE enzyme, or wherein the GE enzyme is coupled to the plasma membrane by a leucine zipper.

4. The modified eukaryotic cell of claim 2, wherein the mucinase is coupled to the plasma membrane.

5. The modified eukaryotic cell of claim 2, wherein the sialidase is coupled to the plasma membrane.

6. The modified eukaryotic cell of claim 2, wherein the mucinase and the sialidase are coupled to the plasma membrane, and wherein optionally the sialidase is coupled to the plasma membrane by a transmembrane domain that is a component of the sialidase, and the mucinase is coupled to the plasma membrane by the leucine zipper.

7. The modified eukaryotic cell of claim 1, wherein the modified eukaryotic cell is a lymphocyte.

8. The modified eukaryotic cell of claim 7, wherein the lymphocyte is a T cell, a natural killer (NK) cell, a natural killer T Cell (NKT) a macrophage, or a dendritic cell.

9. The modified eukaryotic cell of claim 8, wherein the lymphocyte is a T cell or an NK cell.

10. The modified eukaryotic cell of claim 9, wherein the lymphocyte expresses a chimeric antigen receptor (CAR).

11. The modified eukaryotic cell of claim 10, wherein the lymphocyte is T cell.

12. The modified eukaryotic cell of claim 10, wherein the lymphocyte is a NK cell.

13. A method for prophylaxis or treatment of cancer comprising administering to an individual in need thereof modified eukaryotic cells comprising a plasma membrane-coupled glycocalyx-editing (GE) enzyme.

14. The method of claim 13, wherein the GE enzyme is a mucinase or a sialidase.

15. The method of claim 14, wherein the mucinase or the sialidase is coupled to the plasma membrane by a leucine zipper.

16. The method of claim 14, wherein the mucinase is coupled to the plasma membrane.

17. The method of claim 15, wherein the sialidase is coupled to the plasma membrane.

18. The method of claim 14, wherein the mucinase and the sialidase are coupled to the plasma membrane.

19. The method of claim 13, wherein the modified eukaryotic cell is a lymphocyte.

20. The method of claim 19, wherein the lymphocyte is a T cell that is optionally a natural killer T (NKT), a macrophage, a dendritic cell, or a natural killer (NK) cell.

21. The method of claim 20, wherein the lymphocyte is a NKT cell or an NK cell.

22. The method of claim 21, wherein the lymphocyte expresses a chimeric antigen receptor (CAR).

23. The method of claim 22, wherein the lymphocyte is an NK cell.

24. A method of modifying a eukaryotic cell to provide a modified eukaryotic cell comprising a plasma membrane-coupled glycocalyx-editing (GE) enzyme, the method comprising introducing into the eukaryotic cell a first membrane-anchored leucine zipper component, and exposing the cell to a GE enzyme comprising a second leucine zipper component that is cognate to the first leucine zipper component, such that the first and the second leucine zipper components bind to one another, to thereby provide a modified eukaryotic cell comprising the plasma membrane-coupled glycocalyx GE enzyme.

25. The method of claim 24, wherein the eukaryotic cell comprises a lymphocyte selected from the group consisting of T cells that are optionally natural killer T (NKT) cells, natural killer (NK) cells, macrophages, and dendritic cells.

26. A population of modified eukaryotic cells comprising one or more introduced plasma membrane-coupled glycocalyx-editing (GE) enzymes.
